(19) United States
(12) Patent Application Publication
(10) Pub. No.: US 2025/0154202 A1
(43) Pub. Date: May 15, 2025

(54) CYCLIC EPITOPE TAGS

(71) Applicant: BIONTECH SE, Mainz (DE)

(72) Inventors: Zhengxin DONG, Cambridge, MA (US); Jonathan MCGEE, Cambridge, MA (US); Daniel KALLIN, Cambridge, MA (US); Hans-Ulrich SCHMOLDT, Mainz (DE); Joycelyn WÜSTEHUBE-LAUSCH, Mainz (DE); Jan Patrick BOGEN, Mainz (DE); Klaus KOCH, Mainz (DE); Olaf OTHERSEN, Mainz (DE); Christoph KRESS, Mainz (DE)

(21) Appl. No.: 18/834,739

(22) PCT Filed: Feb. 2, 2023
(86) PCT No.: PCT/EP2023/052578

§ 371 Date: Jul. 31, 2024

Related U.S. Application Data
(60) Provisional application No. 63/370,050, filed on Aug. 1, 2022.
(60) Provisional application No. 63/305,905, filed on Feb. 2, 2022.

(30) Foreign Application Priority Data
Feb. 2, 2022 (WO) PCT/EP2022/052465

(51) Int. Cl. C07K 7/64; C07K 16/44

(52) U.S. Cl. CPC C07K 7/64; C07K 16/44; C07K 2317/569; C07K 2317/92; C07K 2319/00

(57) ABSTRACT

The present invention relates to cyclic peptides useful as epitope tags, and compositions thereof. Furthermore, the invention is directed to methods of their use in complex formation.

### BACKGROUND OF THE INVENTION

The present invention relates to cyclic peptides useful as epitope tags, and compositions thereof. Furthermore, the invention is directed to methods of their use in complex formation.

Epitope tagging is a technique in which an epitope is linked to a compound, e.g., a protein. The epitope-tagged compound such as fusion protein can be labelled using an antibody specific for the epitope tag. The antibody may be linked to a further compound, thus creating an interaction between the two compounds. Epitope tags are widely used as tools to detect, purify and manipulate compounds such as proteins. Epitope tagging has proven to be an efficient way to enable immunochemical and immunocytochemical methods on target compounds such as proteins. Epitope tagging can be used for a variety of applications including purification, western blotting, immunoprecipitation, flow cytometry, and immunofluorescence. Epitope tagging may also be used for diagnostic or therapeutic purposes.

Recently, an epitope tag/binder system (ALFA system) comprising the ALFA-tag and an ALFA-specific single-domain antibody (sdAb), NbALFA-nanobody, has been described (Götzke, H. et al, Nat Commun 10, 4403 (2019)). The ALFA system is suited for a broad spectrum of applications and offers a superior and versatile alternative to most common epitope tag systems.

However, it has been found that stability of the ALFA-tag is impaired because the tag is subject to enzymatic cleavage in serum and plasma. This may limit the application of the ALFA system, especially in in vivo settings. Therefore, we set out to modify the ALFA system by adding a cyclic ring to the ALFA-tag that protects the peptide from enzymatic cleavage while maintaining the specificity and favorable biochemical properties of the ALFA system.

### BRIEF SUMMARY OF THE INVENTION

Disclosed is a peptide moiety comprising the cyclized amino acid sequence

-AA0-AA1-AA2-AA3-AA4-AA5-AA6-AA7-AA8-AA9-AA10-AA11-
AA12-AA13-AA14-,

wherein the side-chains of any two of the amino acids of AA, AA1, AA2, AA3, AA4, AA5, AA6, AA7, AA8, AA9, AA10, AA11, AA12, AA13 and AA14 (X1, X2) are connected covalently; and
wherein the amino acids of AA0, AA1, AA2, AA3, AA4, AA5, AA6, AA7, AA8, AA9, AA10, AA11, AA12, AA13 and AA14 which are not X1 and X2 are:
AA0 is Pro or deleted;
AA1 is Ser, Gly, Thr, or Pro;
AA2 is Arg, Gly, Ala, Glu or Pro,
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA10 is Arg;
AA11 is Leu;
AA12 is Thr, Ser, Asp, Glu, Pro, Ala, or deleted;
AA13 is Glu, Lys, Pro, Ser, Ala, Asp, or deleted; and
AA14 is Pro or deleted.

The peptide described herein is useful as an epitope tag. In particular, the peptide described herein is useful as an epitope tag in the ALFA system-epitope tag/binder system comprising an ALFA-specific single-domain antibody (sdAb), NbALFA-nanobody.

### BRIEF DESCRIPTION OF THE DRAWINGS

FIG. 1 shows embodiments of cyclic ALFA peptides demonstrating longer half-lives in human and mouse plasma in in vitro assays

SD 14316:
Ac-Pro-Ser-Arg-Leu-Glu-Glu-Glu-Leu-Arg-Arg-Arg-
Leu-Thr-Glu-NH₂;
SD 14626:
X-Pro-Ser-Arg-Leu-Glu-Glu-Glu-Leu-Arg-Arg-Arg-
Leu-Thr-Glu-NH₂;
SD 15194:
Ac-Ser-Arg-Leu-Glu-(cyclo5)Glu-Glu-Leu-Arg-
(cyclo9)Lys-Arg-Leu-Thr-Glu-NH₂;
SD 15196:
Ac-Ser-Arg-Leu-Glu-(cyclo5)Asp-Glu-Leu-Arg-
(cyclo9)Lys-Arg-Leu-Thr-Glu-NH₂;
SD 15192:
Ac-Ser-Arg-Leu-Glu-(cyclo5)Glu-Glu-Leu-
(cyclo8)Lys-Arg-Arg-Leu-Thr-Glu-NH₂;
SD 15193:
Ac-Ser-Arg-Leu-Glu-Glu-Glu-Leu-Arg-
(cyclo9)Lys-Arg-Leu-Thr-(cyclo13)Glu-NH₂;

FIG. 2 shows embodiments of cyclic peptide retaining the binding affinity to the anti-ALFA VHH with an EC50 of 6.0 nM compared to 2.6 nM of the parent linear ALFA peptide

SD 15197:
Ac-Ser-Arg-Leu-Glu-Glu-Glu-Leu-Arg-Arg-Arg-Leu-
Thr-Glu-NH₂;

FIG. 3 is a UPLC-UV₂₁₄ chromatogram for the peptide Ac-Pro-Ser-Arg-Leu-Glu-cyclo (Glu-Glu-Leu-Arg-Lys)-Arg-Leu-Thr-Glu-NH₂;

FIG. 4 is a mass spectrometer spectrograph for the peptide Ac-Pro-Ser-Arg-Leu-Glu-cyclo (Glu-Glu-Leu-Arg-Lys)-Arg-Leu-Thr-Glu-NH₂;

FIG. 5 illustrates FACS data showing that both linear and cyclic ALFA peptides bind only to target expressing cells previously incubated with the NbALFA×anti-Target Adaptor-Protein;

FIG. 6 is a graph illustrating the NbALFA binding behavior of Ac-ALFA-Amide 2 (SD15197), a linear alpha tag;

FIG. 7 is a graph illustrating the NbALFA binding behavior of Ac-5-8Cyclo (SD15192), a cyclic alpha tag;

FIG. 8 is a graph illustrating the NbALFA binding behavior of Ac-9-13Cyclo (SD15193), a cyclic alpha tag;

FIG. 9 is a graph illustrating the NbALFA binding behavior of Ac-5E-9Cyclo (SD15194), a cyclic alpha tag;

FIG. 10 is a graph illustrating the NbALFA binding behavior of Ac-5D-9Cyclo (SD15196), a cyclic alpha tag;

FIG. 11 is a graph illustrating the NbALFA binding behavior of SD15341, a cyclic alpha tag

SD 15341:
Ac-Pro-Ser-Arg-Leu-Glu-(cyclo6)Glu-Glu-Leu-Arg-
(cyclo10)Lys-Arg-Leu-Thr-Glu-NH₂;

FIG. 12 is a graph illustrating the NbALFA binding behavior of SD15805, a cyclic alpha tag

SD 15805:
Ac-Pro-Ser-Arg-Leu-Glu-(cyclo6)Lys-Glu-Leu-Arg-
(cyclo10)Glu-Arg-Leu-Thr-Glu-NH₂;

FIG. 13 is a graph illustrating the NbALFA binding behavior of SD15808, a cyclic alpha tag

SD 15806:
Ac-Pro-Ser-Arg-Leu-(cyclo5)Glu-Glu-Glu-Leu-
(cyclo9)Lys-Arg-Arg-Leu-Thr-Glu-NH₂;

FIG. 14 is a graph illustrating the NbALFA binding behavior of SD15807, a cyclic alpha tag

SD 15807:
Ac-Pro-Ser-Arg-Leu-Glu-(cyclo6)Cys-Glu-Leu-
(cyclo9)dCys-Arg-Arg-Leu-Thr-Glu-NH₂;

FIG. 15 is a graph illustrating the NbALFA binding behavior of SD15808, a cyclic alpha tag

SD 15808:
Ac-Pro-Ser-Arg-Leu-Glu-(cyclo6)Glu-Glu-Leu-Arg-
(cyclo10)dLys-Arg-Leu-Thr-Glu-NH₂;

FIG. 16 is a graph illustrating comparative plasma concentration (mice) and PK parameters for linear and cyclic alpha tags, and

FIG. 17 is a graph illustrating comparative plasma concentration (mice) and PK parameters for linear and cyclic alpha tags.

### DETAILED DESCRIPTION OF THE INVENTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The practice of the present disclosure will employ, unless otherwise indicated, conventional chemistry, biochemistry, cell biology, immunology, and recombinant DNA techniques which are explained in the literature in the field.

Throughout this specification and the claims which follow, unless the context requires otherwise, the word “comprise”, and variations such as “comprises” and “comprising”, will be understood to imply the inclusion of a stated feature, element, member, integer or step or group of features, elements, members, integers or steps but not the exclusion of any other feature, element, member, integer or step or group of features, elements, members, integers or steps. The term “consisting essentially of” limits the scope of a claim or disclosure to the specified features, elements, members, integers, or steps and those that do not materially affect the basic and novel characteristic(s) of the claim or disclosure. The term “consisting of” limits the scope of a claim or disclosure to the specified features, elements, members, integers, or steps. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

All methods described herein can be performed in any suitable order unless otherwise indicated herein or otherwise clearly contradicted by the context.

The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

The term “optional” or “optionally” as used herein means that the subsequently described event, circumstance or condition nay or may not occur, and that the description includes instances where said event, circumstance, or condition occurs and instances in which it does not occur.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.2%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Disclosed herein are peptides comprising a cyclized peptide sequence for use as an epitope tag and compounds comprising such peptides.

The term “peptide” refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “polypeptide” refers to large peptides, in particular peptides having at least about 151 amino acids. “Peptides” and “polypeptides” are both protein molecules. Thus, the terms “peptide”, “protein” and “polypeptide” are used herein usually as synonyms.

In some embodiments, a peptide comprising a cyclized peptide sequence disclosed herein has a length of about 11 to about 100 amino acids, e.g., about 12 to about 50 amino acids, e.g., about 13 to about 25 amino acids, e.g., about 13, about 14, about 15, about 16, about 17, about 18, or about 19 amino acids.

In some embodiments, the peptides disclosed herein are composed of naturally occurring amino acids, non-naturally occurring amino acids, amino acid derivatives and non-amino acid components, or a mixture thereof. In some embodiments, the peptides disclosed herein comprise amino acid mimetics and amino acid analogs. In some embodiments, the peptides disclosed herein comprise non-naturally occurring amino acid sequences that are resistant to enzymatic cleavage.

In some embodiments, one or more positions of a peptide disclosed herein are substituted with a non-naturally occurring amino acid. In some embodiments, the substituted amino acid is chemically related to the original residue (e.g., aliphatic, charged, basic, acidic, aromatic, hydrophilic) or an isostere of the original residue.

In its broadest sense, as used herein, the term “amino acid” refers to a compound and/or substance that can be, is, or has been incorporated into a peptide, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid. In some embodiments, an amino acid is a D-amino acid. In some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a peptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a peptide containing the modified amino acid as compared with one containing an otherwise identical unmodified amino acid. In some embodiments, such modification does not significantly alter a relevant activity of a peptide containing the modified amino acid, as compared with one containing an otherwise identical unmodified amino acid. As will be clear from context, in some embodiments, the term “amino acid” may be used to refer to a free amino acid. In some embodiments it may be used to refer to an amino acid residue of a peptide.

The following table lists the 20 natural amino acids and their abbreviations:

| Name | Abbr | |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Generally, amino acids are L-amino acids while D-amino acids are denoted by the prefix “D”. The prefix “homo” or “h” designates an α-amino acid that is otherwise similar to one of the common ones, but that contains one more methylene group in the carbon chain.

As used herein, “Orn” means ornithine or 2,5-diaminopentanoic acid, “Dab” means 2,4-diaminobutanoic acid, “Dap” means 2,3-diaminopropanoic acid, “hLys” means 2,7-diaminoheptanoic acid, “hCys” means 2-amino-4-mercaptobutanoic acid, and “Pen” means penicillamine or 2-amino-3-methyl-3-sulfanylbutanoic acid.

It may also be possible to include non-peptide linkages and other chemical modification. For example, part or all of the peptide may be synthesized as a peptidomimetic, e.g., a peptoid (see, e.g., Simon et a. (1992) Proc. Natl. Acad. Sci. USA 89:9367-71 and Horwell (1995) Trends Biotechnol.l3:132-4). A peptide may include one or more (e.g., all) non-hydrolyzable bonds. Many non-hydrolyzable peptide bonds are known in the art, along with procedures for synthesis of peptides containing such bonds. Exemplary non-hydrolyzable bonds include—[CH₂NH]— reduced amide peptide bonds, —[COCH₂]— ketomethylene peptide bonds, —[CH(CN)NH]— (cyanomethylene)amino peptide bonds, —[CH₂CH(OH)]— hydroxyethylene peptide bonds, —[CH₂O]— oxymethylene peptide bonds, and —[CH₂S]— thiomethylene peptide bonds (see e.g., U.S. Pat. No. 6,172,043).

As used herein, an “epitope tag” refers to a peptide to which an antibody or proteinaceous molecule with antibody-like function can bind.

In some embodiments, the peptides, compounds or complexes described herein are isolated.

“Isolated” means removed (e.g., purified) from the natural state or from an artificial composition, such as a composition from a production process. For example, a peptide or polypeptide naturally present in a living animal is not “isolated”, but the same peptide or polypeptide partially or completely separated from the coexisting materials of its natural state is “isolated”. An isolated peptide or polypeptide can exist in substantially purified form, or can exist in a non-native environment such as, for example, a host cell.

The peptide disclosed herein comprises at least one cyclic portion, i.e., a polypeptide chain that contains a circular sequence of bonds that is referred to herein as a “cyclic peptide.” The circular sequence can occur through a connection between the amino and carboxyl ends of the peptide; a connection between the amino end and a side chain; a connection between the carboxyl end and a side chain; or a connection between two side chains including sulfur groups of two cysteine amino acids by forming a disulfide bond, or more complicated arrangements.

The term “amide” as used herein, represents a group of formula “—NHC(O)—”.

The term “thioamide” represents a group of formula “—NHC(S)—”.

As used herein the term “disulfide bond”, “disulfide bridge” or “disulfide” includes the covalent bond formed between two sulfur atoms. The amino acid cysteine comprises a thiol group that can form a disulfide bond or bridge with a second thiol group.

The term “ether” refers to a group or compound having an oxygen between two carbon atoms.

The term “thioether” refers to a group or compound having a sulfur between two carbon atoms.

The term “ester” refers a compound derived from an carboxylic acid and an alcohol by linking with formal loss of water the hydroxyl group of the —C(═O)OH group in the former and a hydroxy group of the latter. Thus, the term refers to the group —C(O)O—.

The term “thioester” refers to the group —C(O)S—.

The term “alkylene” refers to a saturated linear or branched divalent hydrocarbon moiety which may have two to thirty, typically three to twenty, often four to eighteen carbon atoms.

The term “alkenylene” refers to a linear or branched divalent hydrocarbon moiety having at least one carbon carbon double bond in which the total carbon atoms may be two to thirty, typically three to twenty, often four to eighteen.

The term “alkynylene” refers to a linear or branched divalent hydrocarbon moiety having at least one carbon carbon triple bond in which the total carbon atoms may be two to thirty, typically three to twenty, often four to eighteen. Alkynyl groups can optionally have one or more carbon carbon double bonds.

The term “triazole” refers to chemical compounds that incorporate in their structure any heterocyclic structure having a five-membered ring of two carbon atoms and three nitrogen atoms (e.g., 1,2,3-triazole).

In one aspect, disclosed herein is a peptide which comprises the cyclized amino acid sequence

-AA0-AA1-AA2-AA3-AA4-AA5-AA6-AA7-AA8-AA9-AA10-AA11-
AA12-AA13-AA14-,

wherein the side-chains of any two of the amino acids of AA0, AA1, AA2, AA3, AA4, AA5, AA6, AA7, AA8, AA9, AA10, AA11, AA12, AA13 and AA14 (X1, X2) are connected covalently; and
wherein the amino acids of AA0, AA1, AA2, AA3, AA4, AA5, AA6, AA7, AA8, AA9, AA10, AA1, AA12, AA13 and AA14 which are not X1 and X2 are:
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA10 is Arg;
AA11 is Leu;
AA12 is Thr, Ser, Asp, Glu, Pro, Ala, or deleted;
AA13 is Glu, Lys, Pro, Ser, Ala, Asp, or deleted; and
AA14 is Pro or deleted.

In some embodiments, X1 and X2 are separated by 2 or 3 amino acids.

In some embodiments, AA5 is X1 and AA9 is X2, AA5 is X1 and AA8 is X2, AA9 is X1 and AA13 is X2, AA6 is X1 and AA9 is X2, AA is X1 and AA12 is X2, AA10 is X1 and AA13 is X2, AA6 is X1 and AA10 is X2 or AA4 is X1 and AA8 is X2.

In one aspect, disclosed herein is a peptide which comprises a cyclized amino acid sequence selected from the group consisting of

a. -AA0-AA1-AA2-AA3-AA4-cyclo(X1-AA6-AA7-AA8-X2)-
Arg-Leu-AA12-AA13-AA14-,
b. -AA0-AA1-AA2-AA3-AA4-cyclo(X1-AA6-AA7-X2)-AA9-
Arg-Leu-AA12-AA13-AA14-,
c. -AA0-AA1-AA2-AA3-AA4-AA5-AA6-AA7-AA8-cyclo
(X1-Arg-Leu-AA12-X2)-AA14-,
d. -AA0-AA1-AA2-AA3-AA4-AA5-cyclo(X1-AA7-AA8-X2)-
Arg-Leu-AA12-AA13-AA14-,
e. -AA0-AA1-AA2-AA3-AA4-AA5-AA6-AA7-AA8-cyclo
(X1-Arg-Leu-X2)-AA13-AA14-,
f. -AA0-AA1-AA2-AA3-AA4-AA5-AA6-AA7-AA8-AA9-cyclo
(X1-Leu-AA12-X2)-AA14-,
g. -AA0-AA1-AA2-AA3-AA4-AA5-cyclo(X1-AA7-AA8-
AA9-X2)-Leu-AA12-AA13-AA14-,
and
h. -AA0-AA1-AA2-AA3-cyclo(X1-AA5-AA6-AA7-X2)-AA9-
Arg-Leu-AA12-AA13-AA14-,

wherein the side-chains of X₁ and X₂ amino acid residues are connected covalently;
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA12 is Thr, Ser, Asp, Glu, Pro, Ala, or deleted;
AA13 is Glu, Lys, Pro, Ser, Ala, Asp, or deleted; and
AA14 is Pro or deleted.

In some embodiments, X₁ and X₂ in the peptides disclosed herein are connected covalently via an amide, disulfide, thioether, ether, ester, thioester, thioamide, alkylene, alkenylene, alkynylene, and/or 1,2,3-triazole.

In some embodiments, a cyclized amino acid sequence described herein is generated by linking an amino group of a side-chain of one of X₁ and X₂ to the carboxyl group of a side-chain of the other of X₁ and X₂ via an amide bond. The amino group of the side chain of an amino acid that possesses a pendant amine group, e.g., lysine or a lysine derivative, and the carboxyl group of the side chain of an acidic amino acid, e.g., aspartic acid, glutamic acid or a derivative thereof, can be used to generate a cyclized amino acid sequence via an amide bond.

In some embodiments, a cyclized amino acid sequence described herein is generated by linking a sulfhydryl group of a side-chain of one of X₁ and X₂ to the sulfhydryl group of a side-chain of the other of X₁ and X₂ via a disulfide bond. Sulfhydryl group-containing amino acids include cysteine and other sulfhydryl-containing amino acids as Pen.

In some embodiments, X₁ and X₂ are, independently, selected from the group consisting of Glu, DGlu, Asp, DAsp, Lys, DLys, hLys, DhLys, Orn, DOrn, Dab, DDab, Dap, DDap, Cys, DCys, hCys, DhCys, Pen, and DPen, with the proviso that when X₁ is Glu, DGlu, Asp, or DAsp, X₂ is Lys, DLys, hLys, DhLys, Orn, DOrn, Dab, DDab, Dap, or DDap; when X1 is Lys, DLys, hLys, DhLys, Or, DOrn, Dab, DDab, Dap, or DDap, X2 is Glu, DGlu, Asp, or DAsp; and when X1 is Cys, DCys, hCys, DhCys, Pen, or DPen, X2 is Cys, DCys, hCys, DhCys, Pen, or DPen.

In some embodiments, X₁ is Glu and X₂ is Lys. In some embodiments, -cyclo(Glu - - - Lys)-, -c(Glu - - - Lys)-, -cyclo(E - - - K)-, -c(E - - - K)-, -E - - - K- cyclo, or -cycloE - - - cycloK-comprises the following structure:

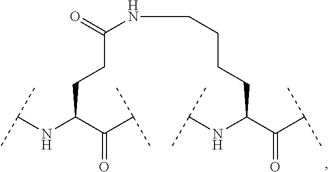

In some embodiments, X₁ is Lys and X₂ is Glu. In some embodiments, -cyclo(Lys - - - Glu)-, -c(Lys - - - Glu)-, -cyclo(K - - - E)-, -c(K - - - E)-, -K - - - E- cyclo, or cycloK - - - cycloE-comprises the following structure:

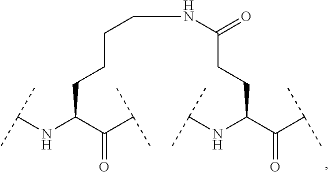

In some embodiments, X₁ is Cys and X₂ is Cys. In some embodiments, -cyclo(Cys - - - Cys)-, c(Cys - - - Cys)-, -cyclo(C - - - C)-, -c(C - - - C)-, -C - - - C- cyclo, or -cycloC - - - cycloC-comprises the following structure:

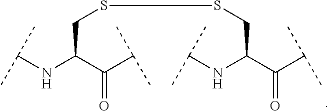

Particular cyclized amino acid sequences of the above-identified generic formulas include, for example,

-Ser-Arg-Leu-Glu-cyclo(Glu-Glu-Leu-Arg-Lys)-Arg-Leu-Thr-Glu-,
-Ser-Arg-Leu-Glu-cyclo(Asp-Glu-Leu-Arg-Lys)-Arg-Leu-Thr-Glu-,
-Ser-Arg-Leu-Glu-cyclo(Glu-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Glu)-,
-Ser-Arg-Leu-Glu-cyclo(Cys-Glu-Leu-Arg-Cys)-Arg-Leu-Thr-Glu-,
-Ser-Arg-Leu-Glu-cyclo(Asp-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Asp)-,
-Ser-Arg-Leu-Glu-cyclo(Glu-Glu-Leu-Arg-DLys)-Arg-Leu-Thr-Glu-,
-Pro-Ser-Arg-Leu-Glu-cyclo(Glu-Glu-Leu-Arg-Lys)-Arg-Leu-Thr-Glu-,
-Pro-Ser-Arg-Leu-Glu-cyclo(DGlu-Glu-Leu-Arg-Lys)-Arg-Leu-Thr-Glu-,
-Pro-Ser-Arg-Leu-Glu-cyclo(Glu-Glu-Leu-Arg-DLys)-Arg-Leu-Thr-Glu-,
-Pro-Ser-Arg-Leu-Glu-cyclo(Lys-Glu-Leu-Arg-Glu)-Arg-Leu-Thr-Glu-,
-Pro-Ser-Arg-Leu-cyclo(Glu-Glu-Glu-Leu-Lys)-Arg-Arg-Leu-Thr-Glu-,
-Pro-Ser-Arg-Leu-Glu-cyclo(Cys-Glu-Leu-DCys)-Arg-Arg-Leu-Thr-Glu-,
-Pro-Ser-Arg-Leu-Glu-Glu-Glu-Leu-Arg-cyclo(Cys-Arg-Leu-Thr-Cys)-,
-Pro-Ser-Arg-Leu-Glu-cyclo(Cys-Glu-Leu-Arg-Cys)-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-cyclo(Cys-Leu-Arg-Arg-Cys)-Leu-Thr-Glu-,
-Ser-Arg-Leu-Glu-Glu-Glu-Leu-Arg-cyclo(Cys-Arg-Leu-Thr-Cys)-,
-Ser-Arg-Leu-Glu-cyclo(Cys-Glu-Leu-Arg-Cys)-Arg-Leu-Thr-Glu-,
-Ser-Arg-Leu-Glu-cyclo(Cys-Glu-Leu-Cys)-Arg-Arg-Leu-Thr-Glu-,
-Ser-Arg-Leu-Glu-Glu-Glu-Leu-Arg-Arg-cyclo(Cys-Leu-Thr-Cys)-,
-Ser-Arg-Leu-Glu-Glu-cyclo(Cys-Leu-Arg-Arg-Cys)-Leu-Thr-Glu-,
-Ser-Arg-Leu-Glu-cyclo(DGlu-Glu-Leu-Arg-DLys)-Arg-Leu-Thr-Glu-,
-Ser-Arg-Leu-Glu-cyclo(DLys-Glu-Leu-Arg-Glu)-Arg-Leu-Thr-Glu-,
-Ser-Arg-Leu-Glu-cyclo(Lys-Glu-Leu-Arg-DGlu)-Arg-Leu-Thr-Glu-,
-Ser-Arg-Leu-Glu-cyclo(DLys-Glu-Leu-Arg-DGlu)-Arg-Leu-Thr-Glu-,
-Pro-Ser-Arg-Leu-Glu-cyclo(DGlu-Glu-Leu-Arg-DLys)-Arg-Leu-Thr-Glu-,
-Pro-Ser-Arg-Leu-Glu-cyclo(DLys-Glu-Leu-Arg-Glu)-Arg-Leu-Thr-Glu-,
-Pro-Ser-Arg-Leu-Glu-cyclo(Lys-Glu-Leu-Arg-DGlu)-Arg-Leu-Thr-Glu-,
-Pro-Ser-Arg-Leu-Glu-cyclo(DLys-Glu-Leu-Arg-DGlu)-Arg-Leu-Thr-Glu-,
-Ser-Arg-Leu-Glu-cyclo(DLys-Glu-Leu-Arg-Asp)-Arg-Leu-Thr-Glu-,
-Ser-Arg-Leu-Glu-cyclo(DAsp-Glu-Leu-Arg-DLys)-Arg-Leu-Thr-Glu-,
-Ser-Arg-Leu-Glu-cyclo(DAsp-Glu-Leu-Arg-Lys)-Arg-Leu-Thr-Glu-,
-Ser-Arg-Leu-Glu-cyclo(Asp-Glu-Leu-Arg-DLys)-Arg-Leu-Thr-Glu-,
-Ser-Arg-Leu-Glu-cyclo(Lys-Glu-Leu-Arg-DAsp)-Arg-Leu-Thr-Glu-,
-Ser-Arg-Leu-Glu-cyclo(DLys-Glu-Leu-Arg-DAsp)-Arg-Leu-Thr-Glu-,
-Pro-Ser-Arg-Leu-Glu-cyclo(DLys-Glu-Leu-Arg-Asp)-Arg-Leu-Thr-Glu-,
-Pro-Ser-Arg-Leu-Glu-cyclo(DAsp-Glu-Leu-Arg-DLys)-Arg-Leu-Thr-Glu-,
-Pro-Ser-Arg-Leu-Glu-cyclo(DAsp-Glu-Leu-Arg-Lys)-Arg-Leu-Thr-Glu-,
-Pro-Ser-Arg-Leu-Glu-cyclo(Asp-Glu-Leu-Arg-DLys)-Arg-Leu-Thr-Glu-,
-Pro-Ser-Arg-Leu-Glu-cyclo(Lys-Glu-Leu-Arg-DAsp)-Arg-Leu-Thr-Glu-,
-Pro-Ser-Arg-Leu-Glu-cyclo(DLys-Glu-Leu-Arg-DAsp)-Arg-Leu-Thr-Glu-,
-Ser-Arg-Leu-Glu-cyclo(DGlu-Glu-Leu-Lys)-Arg-Arg-Leu-Thr-Glu-,
-Ser-Arg-Leu-Glu-cyclo(Glu-Glu-Leu-DLys)-Arg-Arg-Leu-Thr-Glu-,
-Ser-Arg-Leu-Glu-cyclo(DGlu-Glu-Leu-DLys)-Arg-Arg-Leu-Thr-Glu-,
-Ser-Arg-Leu-Glu-cyclo(Lys-Glu-Leu-Glu)-Arg-Arg-Leu-Thr-Glu-,
-Ser-Arg-Leu-Glu-cyclo(Lys-Glu-Leu-DGlu)-Arg-Arg-Leu-Thr-Glu-,
-Ser-Arg-Leu-Glu-cyclo(DLys-Glu-Leu-DGlu)-Arg-Arg-Leu-Thr-Glu-,
-Ser-Arg-Leu-Glu-cyclo(Asp-Glu-Leu-Lys)-Arg-Arg-Leu-Thr-Glu-,
-Ser-Arg-Leu-Glu-cyclo(Asp-Glu-Leu-DLys)-Arg-Arg-Leu-Thr-Glu-,
-Ser-Arg-Leu-Glu-cyclo(Lys-Glu-Leu-Asp)-Arg-Arg-Leu-Thr-Glu-,
-Ser-Arg-Leu-Glu-cyclo(Lys-Glu-Leu-DAsp)-Arg-Arg-Leu-Thr-Glu-,
-Ser-Arg-Leu-Glu-cyclo(DAsp-Glu-Leu-Lys)-Arg-Arg-Leu-Thr-Glu-,
-Ser-Arg-Leu-Glu-cyclo(DAsp-Glu-Leu-DLys)-Arg-Arg-Leu-Thr-Glu-,
-Ser-Arg-Leu-Glu-cyclo(DLys-Glu-Leu-DAsp)-Arg-Arg-Leu-Thr-Glu-,
-Ser-Arg-Leu-Glu-cyclo(DLys-Glu-Leu-Asp)-Arg-Arg-Leu-Thr-Glu-,
-Pro-Ser-Arg-Leu-Glu-cyclo(Glu-Glu-Leu-DLys)-Arg-Arg-Leu-Thr-Glu-,
-Pro-Ser-Arg-Leu-Glu-cyclo(DGlu-Glu-Leu-DLys)-Arg-Arg-Leu-Thr-Glu-
-Pro-Ser-Arg-Leu-Glu-cyclo(Lys-Glu-Leu-Glu)-Arg-Arg-Leu-Thr-Glu-,
-Pro-Ser-Arg-Leu-Glu-cyclo(Lys-Glu-Leu-DGlu)-Arg-Arg-Leu-Thr-Glu-,
-Pro-Ser-Arg-Leu-Glu-cyclo(DLys-Glu-Leu-DGlu)-Arg-Arg-Leu-Thr-Glu-,
-Pro-Ser-Arg-Leu-Glu-cyclo(Asp-Glu-Leu-Lys)-Arg-Arg-Leu-Thr-Glu-,
-Pro-Ser-Arg-Leu-Glu-cyclo(Asp-Glu-Leu-DLys)-Arg-Arg-Leu-Thr-Glu-,
-Pro-Ser-Arg-Leu-Glu-cyclo(Lys-Glu-Leu-Asp)-Arg-Arg-Leu-Thr-Glu-,
-Pro-Ser-Arg-Leu-Glu-cyclo(Lys-Glu-Leu-DAsp)-Arg-Arg-Leu-Thr-Glu-,
-Pro-Ser-Arg-Leu-Glu-cyclo(DAsp-Glu-Leu-Lys)-Arg-Arg-Leu-Thr-Glu-,
-Pro-Ser-Arg-Leu-Glu-cyclo(DAsp-Glu-Leu-DLys)-Arg-Arg-Leu-Thr-Glu-,
-Pro-Ser-Arg-Leu-Glu-cyclo(DLys-Glu-Leu-DAsp)-Arg-Arg-Leu-Thr-Glu-,
-Pro-Ser-Arg-Leu-Glu-cyclo(DLys-Glu-Leu-Asp)-Arg-Arg-Leu-Thr-Glu-,
-Ser-Arg-Leu-Glu-Glu-cyclo(Asp-Leu-Arg-Lys)-Arg-Leu-Thr-Glu-,
-Ser-Arg-Leu-Glu-Glu-cyclo(Lys-Leu-Arg-Asp)-Arg-Leu-Thr-Glu-,
-Ser-Arg-Leu-Glu-Glu-cyclo(DAsp-Leu-Arg-Lys)-Arg-Leu-Thr-Glu-,
-Ser-Arg-Leu-Glu-Glu-cyclo(Lys-Leu-Arg-DAsp)-Arg-Leu-Thr-Glu-,
-Ser-Arg-Leu-Glu-Glu-cyclo(Asp-Leu-Arg-DLys)-Arg-Leu-Thr-Glu-,
-Ser-Arg-Leu-Glu-Glu-cyclo(DLys-Leu-Arg-Asp)-Arg-Leu-Thr-Glu-,
-Ser-Arg-Leu-Glu-Glu-cyclo(DLys-Leu-Arg-DAsp)-Arg-Leu-Thr-Glu-,
-Ser-Arg-Leu-Glu-Glu-cyclo(DAsp-Leu-Arg-DLys)-Arg-Leu-Thr-Glu-,
-Pro-Ser-Arg-Leu-Glu-Glu-cyclo(Asp-Leu-Arg-Lys)-Arg-Leu-Thr-Glu-,
-Pro-Ser-Arg-Leu-Glu-Glu-cyclo(Lys-Leu-Arg-Asp)-Arg-Leu-Thr-Glu-,
-Pro-Ser-Arg-Leu-Glu-Glu-cyclo(DAsp-Leu-Arg-Lys)-Arg-Leu-Thr-Glu-,
-Pro-Ser-Arg-Leu-Glu-Glu-cyclo(Lys-Leu-Arg-DAsp)-Arg-Leu-Thr-Glu-,
-Pro-Ser-Arg-Leu-Glu-Glu-cyclo(Asp-Leu-Arg-DLys)-Arg-Leu-Thr-Glu-,
-Pro-Ser-Arg-Leu-Glu-Glu-cyclo(DLys-Leu-Arg-Asp)-Arg-Leu-Thr-Glu-,
-Pro-Ser-Arg-Leu-Glu-Glu-cyclo(DLys-Leu-Arg-DAsp)-Arg-Leu-Thr-Glu-,
-Pro-Ser-Arg-Leu-Glu-Glu-cyclo(DAsp-Leu-Arg-DLys)-Arg-Leu-Thr-Glu-,
-Ser-Arg-Leu-Glu-cyclo(Cys-Glu-Leu-Cys)-Arg-Arg-Leu-Thr-Glu-,
-Ser-Arg-Leu-Glu-Glu-Glu-Leu-Arg-Arg-cyclo(Cys-Leu-Thr-Cys)-,
-Ser-Arg-Leu-Glu-Glu-Glu-Leu-Arg-cyclo(Cys-Arg-Leu-Cys)-Glu-,
-Ser-Arg-Leu-Glu-Glu-cyclo(Cys-Leu-Arg-Cys)-Arg-Leu-Thr-Glu-,
-Pro-Ser-Arg-Leu-Glu-cyclo(Cys-Glu-Leu-Cys)-Arg-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-Glu-Leu-Arg-cyclo(Cys-Arg-Leu-Cys)-Glu-,
-Pro-Ser-Arg-Leu-Glu-Glu-cyclo(Cys-Leu-Arg-Cys)-Arg-Leu-Thr-Glu-,
-Ser-Arg-Leu-Glu-cyclo(DCys-Glu-Leu-DCys)-Arg-Arg-Leu-Thr-Glu-,
-Ser-Arg-Leu-Glu-cyclo(DCys-Glu-Leu-Cys)-Arg-Arg-Leu-Thr-Glu-,
-Ser-Arg-Leu-Glu-cyclo(hCys-Glu-Leu-Cys)-Arg-Arg-Leu-Thr-Glu-,
-Ser-Arg-Leu-Glu-cyclo(hCys-Glu-Leu-hCys)-Arg-Arg-Leu-Thr-Glu-,
-Ser-Arg-Leu-Glu-cyclo(Cys-Glu-Leu-hCys)-Arg-Arg-Leu-Thr-Glu-,
-Ser-Arg-Leu-Glu-cyclo(hCys-Glu-Leu-DCys)-Arg-Arg-Leu-Thr-Glu-,
-Ser-Arg-Leu-Glu-cyclo(DCys-Glu-Leu-hCys)-Arg-Arg-Leu-Thr-Glu-,
-Ser-Arg-Leu-Glu-cyclo(hCys-Glu-Leu-DhCys)-Arg-Arg-Leu-Thr-Glu-,
-Ser-Arg-Leu-Glu-cyclo(DhCys-Glu-Leu-DhCys)-Arg-Arg-Leu-Thr-Glu-,
-Ser-Arg-Leu-Glu-cyclo(Cys-Glu-Leu-DhCys)-Arg-Arg-Leu-Thr-Glu-,
-Ser-Arg-Leu-Glu-cyclo(DCys-Glu-Leu-DhCys)-Arg-Arg-Leu-Thr-Glu-,
-Ser-Arg-Leu-Glu-cyclo(DhCys-Glu-Leu-Cys)-Arg-Arg-Leu-Thr-Glu-,
-Ser-Arg-Leu-Glu-cyclo(DhCys-Glu-Leu-DCys)-Arg-Arg-Leu-Thr-Glu-,
-Pro-Ser-Arg-Leu-Glu-cyclo(DCys-Glu-Leu-DCys)-Arg-Arg-Leu-Thr-Glu-,
-Pro-Ser-Arg-Leu-Glu-cyclo(DCys-Glu-Leu-Cys)-Arg-Arg-Leu-Thr-Glu-,
-Pro-Ser-Arg-Leu-Glu-cyclo(hCys-Glu-Leu-Cys)-Arg-Arg-Leu-Thr-Glu-,
-Pro-Ser-Arg-Leu-Glu-cyclo(hCys-Glu-Leu-hCys)-Arg-Arg-Leu-Thr-Glu-,
-Pro-Ser-Arg-Leu-Glu-cyclo(Cys-Glu-Leu-hCys)-Arg-Arg-Leu-Thr-Glu-,
-Pro-Ser-Arg-Leu-Glu-cyclo(hCys-Glu-Leu-DCys)-Arg-Arg-Leu-Thr-Glu-,
-Pro-Ser-Arg-Leu-Glu-cyclo(DCys-Glu-Leu-hCys)-Arg-Arg-Leu-Thr-Giu-,
-Pro-Ser-Arg-Leu-Glu-cyclo(hCys-Glu-Leu-DhCys)-Arg-Arg-Leu-Thr-Glu-,
-Pro-Ser-Arg-Leu-Glu-cyclo(DhCys-Glu-Leu-DhCys)-Arg-Arg-Leu-Thr-Glu-,
-Pro-Ser-Arg-Leu-Glu-cyclo(Cys-Glu-Leu-DhCys)-Arg-Arg-Leu-Thr-Glu-,
-Pro-Ser-Arg-Leu-Glu-cyclo(DCys-Glu-Leu-DhCys)-Arg-Arg-Leu-Thr-Glu-,
-Pro-Ser-Arg-Leu-Glu-cyclo(DhCys-Glu-Leu-Cys)-Arg-Arg-Leu-Thr-Glu-,
-Pro-Ser-Arg-Leu-Glu-cyclo(DhCys-Glu-Leu-DCys)-Arg-Arg-Leu-Thr-Glu-,
-Ser-Arg-Leu-Glu-Glu-Glu-Leu-Arg-Arg-cyclo(Cys-Leu-Thr-DCys)-,
-Ser-Arg-Leu-Glu-Glu-Glu-Leu-Arg-Arg-cyclo(DCys-Leu-Thr-Cys)-,
-Ser-Arg-Leu-Glu-Glu-Glu-Leu-Arg-Arg-cyclo(DCys-Leu-Thr-DCys)-,
-Ser-Arg-Leu-Glu-Glu-Glu-Leu-Arg-Arg-cyclo(hCys-Leu-Thr-Cys)-,
-Ser-Arg-Leu-Glu-Glu-Glu-Leu-Arg-Arg-cyclo(hCys-Leu-Thr-DCys)-,
-Ser-Arg-Leu-Glu-Glu-Glu-Leu-Arg-Arg-cyclo(hCys-Leu-Thr-hCys)-,
-Ser-Arg-Leu-Glu-Glu-Glu-Leu-Arg-Arg-cyclo(Cys-Leu-Thr-hCys)-,
-Ser-Arg-Leu-Glu-Glu-Glu-Leu-Arg-Arg-cyclo(DCys-Leu-Thr-hCys)-,
-Ser-Arg-Leu-Glu-Glu-Glu-Leu-Arg-Arg-cyclo(Cys-Leu-Thr-hCys)-,
-Ser-Arg-Leu-Glu-Glu-Glu-Leu-Arg-Arg-cyclo(DhCys-Leu-Thr-Cys)-,
-Ser-Arg-Leu-Glu-Glu-Glu-Leu-Arg-Arg-cyclo(DhCys-Leu-Thr-DCys)-,
-Ser-Arg-Leu-Glu-Glu-Glu-Leu-Arg-Arg-cyclo(DhCys-Leu-Thr-hCys)-,
-Ser-Arg-Leu-Glu-Glu-Glu-Leu-Arg-Arg-cyclo(Cys-Leu-Thr-DhCys)-,
-Ser-Arg-Leu-Glu-Glu-Glu-Leu-Arg-Arg-cyclo(DCys-Leu-Thr-DhCys)-,
-Ser-Arg-Leu-Glu-Glu-Glu-Leu-Arg-Arg-cyclo(Cys-Leu-Thr-DhCys)-,
-Ser-Arg-Leu-Glu-Glu-Glu-Leu-Arg-Arg-cyclo(Pen-Leu-Thr-Pen)-,
-Ser-Arg-Leu-Glu-Glu-Glu-Leu-Arg-Arg-cyclo(Pen-Leu-Thr-DPen)-,
-Ser-Arg-Leu-Glu-Glu-Glu-Leu-Arg-Arg-cyclo(DPen-Leu-Thr-Pen)-,
-Ser-Arg-Leu-Glu-Glu-Glu-Leu-Arg-Arg-cyclo(DPen-Leu-Thr-DPen)-,
-Ser-Arg-Leu-Glu-Glu-Glu-Leu-Arg-cyclo(DCys-Arg-Leu-Cys)-Glu-,
-Ser-Arg-Leu-Glu-Glu-Glu-Leu-Arg-cyclo(DCys-Arg-Leu-DCys)-Glu-,
-Ser-Arg-Leu-Glu-Glu-Glu-Leu-Arg-cyclo(Cys-Arg-Leu-DCys)-Glu-,
-Ser-Arg-Leu-Glu-Glu-Glu-Leu-Arg-cyclo(hCys-Arg-Leu-Cys)-Glu-,
-Ser-Arg-Leu-Glu-Glu-Glu-Leu-Arg-cyclo(hCys-Arg-Leu-DCys)-Glu-,
-Ser-Arg-Leu-Glu-Glu-Glu-Leu-Arg-cyclo(Cys-Arg-Leu-hCys)-Glu-,
-Ser-Arg-Leu-Glu-Glu-Glu-Leu-Arg-cyclo(hCys-Arg-Leu-hCys)-Glu-,
-Ser-Arg-Leu-Glu-Glu-Glu-Leu-Arg-cyclo(hCys-Arg-Leu-DhCys)-Glu-,
-Ser-Arg-Leu-Glu-Glu-Glu-Leu-Arg-cyclo(DhCys-Arg-Leu-hCys)-Glu-,
-Pro-Ser-Arg-Leu-Glu-Glu-Glu-Leu-Arg-Arg-cyclo(Cys-Leu-Thr-DCys)-,
-Pro-Ser-Arg-Leu-Glu-Glu-Glu-Leu-Arg-Arg-cyclo(DCys-Leu-Thr-Cys)-,
-Pro-Ser-Arg-Leu-Glu-Glu-Glu-Leu-Arg-Arg-cyclo(DCys-Leu-Thr-DCys)-,
-Pro-Ser-Arg-Leu-Glu-Glu-Glu-Leu-Arg-Arg-cyclo(hCys-Leu-Thr-Cys)-,
-Pro-Ser-Arg-Leu-Glu-Glu-Glu-Leu-Arg-Arg-cyclo(hCys-Leu-Thr-DCys)-,
-Pro-Ser-Arg-Leu-Glu-Glu-Glu-Leu-Arg-Arg-cyclo(hCys-Leu-Thr-hCys)-,
-Pro-Ser-Arg-Leu-Glu-Glu-Glu-Leu-Arg-Arg-cyclo(Cys-Leu-Thr-hCys)-,
-Pro-Ser-Arg-Leu-Glu-Glu-Glu-Leu-Arg-Arg-cyclo(DCys-Leu-Thr-hCys)-,
-Pro-Ser-Arg-Leu-Glu-Glu-Glu-Leu-Arg-Arg-cyclo(Cys-Leu-Thr-hCys)-,
-Pro-Ser-Arg-Leu-Glu-Glu-Glu-Leu-Arg-Arg-cyclo(DhCys-Leu-Thr-Cys)-,
-Pro-Ser-Arg-Leu-Glu-Glu-Glu-Leu-Arg-Arg-cyclo(DhCys-Leu-Thr-DCys)-,
-Pro-Ser-Arg-Leu-Glu-Glu-Glu-Leu-Arg-Arg-cyclo(DhCys-Leu-Thr-hCys)-,
-Pro-Ser-Arg-Leu-Glu-Glu-Glu-Leu-Arg-Arg-cyclo(Cys-Leu-Thr-DhCys)-,
-Pro-Ser-Arg-Leu-Glu-Glu-Glu-Leu-Arg-Arg-cyclo(DCys-Leu-Thr-DhCys)-,
-Pro-Ser-Arg-Leu-Glu-Glu-Glu-Leu-Arg-Arg-cyclo(Cys-Leu-Thr-DhCys)-,
-Pro-Ser-Arg-Leu-Glu-Glu-Glu-Leu-Arg-Arg-cyclo(Pen-Leu-Thr-Pen)-,
-Pro-Ser-Arg-Leu-Glu-Glu-Glu-Leu-Arg-Arg-cyclo(Pen-Leu-Thr-DPen)-,
-Pro-Ser-Arg-Leu-Glu-Glu-Glu-Leu-Arg-Arg-cyclo(DPen-Leu-Thr-Pen)-,
-Pro-Ser-Arg-Leu-Glu-Glu-Glu-Leu-Arg-Arg-cyclo(DPen-Leu-Thr-DPen)-,
-Pro-Ser-Arg-Leu-Glu-Glu-Glu-Leu-Arg-cyclo(DCys-Arg-Leu-Cys)-Glu-,
-Pro-Ser-Arg-Leu-Glu-Glu-Glu-Leu-Arg-cyclo(DCys-Arg-Leu-DCys)-Glu-,
-Pro-Ser-Arg-Leu-Glu-Glu-Glu-Leu-Arg-cyclo(Cys-Arg-Leu-DCys)-Glu-,
-Pro-Ser-Arg-Leu-Glu-Glu-Glu-Leu-Arg-cyclo(hCys-Arg-Leu-Cys)-Glu-,
-Pro-Ser-Arg-Leu-Glu-Glu-Glu-Leu-Arg-cyclo(hCys-Arg-Leu-DCys)-Glu-,
-Pro-Ser-Arg-Leu-Glu-Glu-Glu-Leu-Arg-cyclo(Cys-Arg-Leu-hCys)-Glu-,
-Pro-Ser-Arg-Leu-Glu-Glu-Glu-Leu-Arg-cyclo(hCys-Arg-Leu-hCys)-Glu-,
-Pro-Ser-Arg-Leu-Glu-Glu-Glu-Leu-Arg-cyclo(hCys-Arg-Leu-DhCys)-Glu-,
-Pro-Ser-Arg-Leu-Glu-Glu-Glu-Leu-Arg-cyclo(DhCys-Arg-Leu-hCys)-Glu-,
-Ser-Arg-Leu-Glu-Glu-Glu-Leu-Arg-cyclo(Lys-Arg-Leu-Asp)-Glu-,
-Ser-Arg-Leu-Glu-Glu-Glu-Leu-Arg-cyclo(Asp-Arg-Leu-Lys)-Glu-,
-Ser-Arg-Leu-Glu-Glu-Glu-Leu-Arg-cyclo(Lys-Arg-Leu-Glu)-Glu-,
-Ser-Arg-Leu-Glu-Glu-Glu-Leu-Arg-cyclo(Glu-Arg-Leu-Lys)-Glu-,
-Pro-Ser-Arg-Leu-Glu-Glu-Glu-Leu-Arg-cyclo(Lys-Arg-Leu-Asp)-Glu-,
-Pro-Ser-Arg-Leu-Glu-Glu-Glu-Leu-Arg-cyclo(Asp-Arg-Leu-Lys)-Glu-,
-Pro-Ser-Arg-Leu-Glu-Glu-Glu-Leu-Arg-cyclo(Lys-Arg-Leu-Glu)-Glu-,
-Pro-Ser-Arg-Leu-Glu-Glu-Glu-Leu-Arg-cyclo(Glu-Arg-Leu-Lys)-Glu-,
-Pro-Ser-Arg-Leu-Glu-Glu-Glu-Leu-Arg-cyclo(Cys-Arg-Leu-Thr-Cys)-,
-Pro-Ser-Arg-Leu-Glu-cyclo(Cys-Glu-Leu-Arg-Cys)-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-cyclo(Cys-Leu-Arg-Arg-Cys)-Leu-Thr-Glu-,
-Ser-Arg-Leu-Glu-cyclo(DGlu-Glu-Leu-Arg-DLys)-Arg-Leu-Thr-Glu-Pro-,
-Ser-Arg-Leu-Glu-cyclo(DLys-Glu-Leu-Arg-Glu)-Arg-Leu-Thr-Glu-Pro-,
-Ser-Arg-Leu-Glu-cyclo(Lys-Glu-Leu-Arg-DGlu)-Arg-Leu-Thr-Glu-Pro-,
-Ser-Arg-Leu-Glu-cyclo(DLys-Glu-Leu-Arg-DGlu)-Arg-Leu-Thr-Glu-Pro-,
-Pro-Ser-Arg-Leu-Glu-cyclo(DGlu-Glu-Leu-Arg-DLys)-Arg-Leu-Thr-Glu-Pro-,
-Pro-Ser-Arg-Leu-Glu-cyclo(DLys-Glu-Leu-Arg-Glu)-Arg-Leu-Thr-Glu-Pro-,
-Pro-Ser-Arg-Leu-Glu-cyclo(Lys-Glu-Leu-Arg-DGlu)-Arg-Leu-Thr-Glu-Pro-,
-Pro-Ser-Arg-Leu-Glu-cyclo(DLys-Glu-Leu-Arg-DGlu)-Arg-Leu-Thr-Glu-Pro-,
-Ser-Arg-Leu-Glu-cyclo(DLys-Glu-Leu-Arg-Asp)-Arg-Leu-Thr-Glu-Pro-,
-Ser-Arg-Leu-Glu-cyclo(DAsp-Glu-Leu-Arg-DLys)-Arg-Leu-Thr-Glu-Pro-,
-Ser-Arg-Leu-Glu-cyclo(DAsp-Glu-Leu-Arg-Lys)-Arg-Leu-Thr-Glu-Pro-,
-Ser-Arg-Leu-Glu-cyclo(Asp-Glu-Leu-Arg-DLys)-Arg-Leu-Thr-Glu-Pro-,
-Ser-Arg-Leu-Glu-cyclo(Lys-Glu-Leu-Arg-DAsp)-Arg-Leu-Thr-Glu-Pro-,
-Ser-Arg-Leu-Glu-cyclo(DLys-Glu-Leu-Arg-DAsp)-Arg-Leu-Thr-Glu-Pro-,
-Pro-Ser-Arg-Leu-Glu-cyclo(DLys-Glu-Leu-Arg-Asp)-Arg-Leu-Thr-Glu-Pro-,
-Pro-Ser-Arg-Leu-Glu-cyclo(DAsp-Glu-Leu-Arg-DLys)-Arg-Leu-Thr-Glu-Pro-,
-Pro-Ser-Arg-Leu-Glu-cyclo(DAsp-Glu-Leu-Arg-Lys)-Arg-Leu-Thr-Glu-Pro-,
-Pro-Ser-Arg-Leu-Glu-cyclo(Asp-Glu-Leu-Arg-DLys)-Arg-Leu-Thr-Glu-Pro-,
-Pro-Ser-Arg-Leu-Glu-cyclo(Lys-Glu-Leu-Arg-DAsp)-Arg-Leu-Thr-Glu-Pro-,
-Pro-Ser-Arg-Leu-Glu-cyclo(DLys-Glu-Leu-Arg-DAsp)-Arg-Leu-Thr-Glu-Pro-,
-Ser-Arg-Leu-Glu-cyclo(DGlu-Glu-Leu-Lys)-Arg-Arg-Leu-Thr-Glu-Pro-,
-Ser-Arg-Leu-Glu-cyclo(Glu-Glu-Leu-DLys)-Arg-Arg-Leu-Thr-Glu-Pro-,
-Ser-Arg-Leu-Glu-cyclo(DGlu-Glu-Leu-DLys)-Arg-Arg-Leu-Thr-Glu-Pro-,
-Ser-Arg-Leu-Glu-cyclo(Lys-Glu-Leu-Glu)-Arg-Arg-Leu-Thr-Glu-Pro-,
-Ser-Arg-Leu-Glu-cyclo(Lys-Glu-Leu-DGlu)-Arg-Arg-Leu-Thr-Glu-Pro-,
-Ser-Arg-Leu-Glu-cyclo(DLys-Glu-Leu-DGlu)-Arg-Arg-Leu-Thr-Glu-Pro-,
-Ser-Arg-Leu-Glu-cyclo(Asp-Glu-Leu-Lys)-Arg-Arg-Leu-Thr-Glu-Pro-,
-Ser-Arg-Leu-Glu-cyclo(Asp-Glu-Leu-DLys)-Arg-Arg-Leu-Thr-Glu-Pro-,
-Ser-Arg-Leu-Glu-cyclo(Lys-Glu-Leu-Asp)-Arg-Arg-Leu-Thr-Glu-Pro-,
-Ser-Arg-Leu-Glu-cyclo(Lys-Glu-Leu-DAsp)-Arg-Arg-Leu-Thr-Glu-Pro-,
-Ser-Arg-Leu-Glu-cyclo(DAsp-Glu-Leu-Lys)-Arg-Arg-Leu-Thr-Glu-Pro-,
-Ser-Arg-Leu-Glu-cyclo(DAsp-Glu-Leu-DLys)-Arg-Arg-Leu-Thr-Glu-Pro-,
-Ser-Arg-Leu-Glu-cyclo(DLys-Glu-Leu-DAsp)-Arg-Arg-Leu-Thr-Glu-Pro-,
-Ser-Arg-Leu-Glu-cyclo(DLys-Glu-Leu-Asp)-Arg-Arg-Leu-Thr-Glu-Pro-,
-Pro-Ser-Arg-Leu-Glu-cyclo(Glu-Glu-Leu-DLys)-Arg-Arg-Leu-Thr-Glu-Pro-,
-Pro-Ser-Arg-Leu-Glu-cyclo(DGlu-Glu-Leu-DLys)-Arg-Arg-Leu-Thr-Glu-Pro-,
-Pro-Ser-Arg-Leu-Glu-cyclo(Lys-Glu-Leu-Glu)-Arg-Arg-Leu-Thr-Glu-Pro-,
-Pro-Ser-Arg-Leu-Glu-cyclo(Lys-Glu-Leu-DGlu)-Arg-Arg-Leu-Thr-Glu-Pro-,
-Pro-Ser-Arg-Leu-Glu-cyclo(DLys-Glu-Leu-DGlu)-Arg-Arg-Leu-Thr-Glu-Pro-,
-Pro-Ser-Arg-Leu-Glu-cyclo(Asp-Glu-Leu-Lys)-Arg-Arg-Leu-Thr-Glu-Pro-,
-Pro-Ser-Arg-Leu-Glu-cyclo(Asp-Glu-Leu-DLys)-Arg-Arg-Leu-Thr-Glu-Pro-,
-Pro-Ser-Arg-Leu-Glu-cyclo(Lys-Glu-Leu-Asp)-Arg-Arg-Leu-Thr-Glu-Pro-,
-Pro-Ser-Arg-Leu-Glu-cyclo(Lys-Glu-Leu-DAsp)-Arg-Arg-Leu-Thr-Glu-Pro-,
-Pro-Ser-Arg-Leu-Glu-cyclo(DAsp-Glu-Leu-Lys)-Arg-Arg-Leu-Thr-Glu-Pro-,
-Pro-Ser-Arg-Leu-Glu-cyclo(DAsp-Glu-Leu-DLys)-Arg-Arg-Leu-Thr-Glu-Pro-,
-Pro-Ser-Arg-Leu-Glu-cyclo(DLys-Glu-Leu-DAsp)-Arg-Arg-Leu-Thr-Glu-Pro-,
-Pro-Ser-Arg-Leu-Glu-cyclo(DLys-Glu-Leu-Asp)-Arg-Arg-Leu-Thr-Glu-Pro-,
-Ser-Arg-Leu-Glu-Glu-cyclo(Asp-Leu-Arg-Lys)-Arg-Leu-Thr-Glu-Pro-,
-Ser-Arg-Leu-Glu-Glu-cyclo(Lys-Leu-Arg-Asp)-Arg-Leu-Thr-Glu-Pro-,
-Ser-Arg-Leu-Glu-Glu-cyclo(DAsp-Leu-Arg-Lys)-Arg-Leu-Thr-Glu-Pro-,
-Ser-Arg-Leu-Glu-Glu-cyclo(Lys-Leu-Arg-DAsp)-Arg-Leu-Thr-Glu-Pro-,
-Ser-Arg-Leu-Glu-Glu-cyclo(Asp-Leu-Arg-DLys)-Arg-Leu-Thr-Glu-Pro-,
-Ser-Arg-Leu-Glu-Glu-cyclo(DLys-Leu-Arg-Asp)-Arg-Leu-Thr-Glu-Pro-,
-Ser-Arg-Leu-Glu-Glu-cyclo(DLys-Leu-Arg-DAsp)-Arg-Leu-Thr-Glu-Pro-,
-Ser-Arg-Leu-Glu-Glu-cyclo(DAsp-Leu-Arg-DLys)-Arg-Leu-Thr-Glu-Pro-,
-Pro-Ser-Arg-Leu-Glu-Glu-cyclo(Asp-Leu-Arg-Lys)-Arg-Leu-Thr-Glu-Pro-,
-Pro-Ser-Arg-Leu-Glu-Glu-cyclo(Lys-Leu-Arg-Asp)-Arg-Leu-Thr-Glu-Pro-,
-Pro-Ser-Arg-Leu-Glu-Glu-cyclo(DAsp-Leu-Arg-Lys)-Arg-Leu-Thr-Glu-Pro-,
-Pro-Ser-Arg-Leu-Glu-Glu-cyclo(Lys-Leu-Arg-DAsp)-Arg-Leu-Thr-Glu-Pro-,
-Pro-Ser-Arg-Leu-Glu-Glu-cyclo(Asp-Leu-Arg-DLys)-Arg-Leu-Thr-Glu-Pro-,
-Pro-Ser-Arg-Leu-Glu-Glu-cyclo(DLys-Leu-Arg-Asp)-Arg-Leu-Thr-Glu-Pro-,
-Pro-Ser-Arg-Leu-Glu-Glu-cyclo(DLys-Leu-Arg-DAsp)-Arg-Leu-Thr-Glu-Pro-,
-Pro-Ser-Arg-Leu-Glu-Glu-cyclo(DAsp-Leu-Arg-DLys)-Arg-Leu-Thr-Glu-Pro-,
-Ser-Arg-Leu-Glu-cyclo(Cys-Glu-Leu-Cys)-Arg-Arg-Leu-Thr-Glu-Pro-,
-Ser-Arg-Leu-Glu-Glu-Glu-Leu-Arg-Arg-cyclo(Cys-Leu-Thr-Cys)-Pro-,
-Ser-Arg-Leu-Glu-Glu-Glu-Leu-Arg-cyclo(Cys-Arg-Leu-Cys)-Glu-Pro-,
-Ser-Arg-Leu-Glu-Glu-cyclo(Cys-Leu-Arg-Cys)-Arg-Leu-Thr-Glu-Pro-,
-Pro-Ser-Arg-Leu-Glu-cyclo(Cys-Glu-Leu-Cys)-Arg-Arg-Leu-Thr-Glu-Pro-,
-Pro-Ser-Arg-Leu-Glu-Glu-Glu-Leu-Arg-Arg-cyclo(Cys-Leu-Thr-Cys)-Pro-,
-Pro-Ser-Arg-Leu-Glu-Glu-Glu-Leu-Arg-cyclo(Cys-Arg-Leu-Cys)-Glu-Pro-,
-Pro-Ser-Arg-Leu-Glu-Glu-cyclo(Cys-Leu-Arg-Cys)-Arg-Leu-Thr-Glu-Pro-,
-Ser-Arg-Leu-Glu-cyclo(DCys-Glu-Leu-DCys)-Arg-Arg-Leu-Thr-Glu-Pro-,
-Ser-Arg-Leu-Glu-cyclo(DCys-Glu-Leu-Cys)-Arg-Arg-Leu-Thr-Glu-Pro-,
-Ser-Arg-Leu-Glu-cyclo(hCys-Glu-Leu-Cys)-Arg-Arg-Leu-Thr-Glu-Pro-,
-Ser-Arg-Leu-Glu-cyclo(hCys-Glu-Leu-hCys)-Arg-Arg-Leu-Thr-Glu-Pro-,
-Ser-Arg-Leu-Glu-cyclo(Cys-Glu-Leu-hCys)-Arg-Arg-Leu-Thr-Glu-Pro-,
-Ser-Arg-Leu-Glu-cyclo(hCys-Glu-Leu-DCys)-Arg-Arg-Leu-Thr-Glu-Pro-,
-Ser-Arg-Leu-Glu-cyclo(DCys-Glu-Leu-hCys)-Arg-Arg-Leu-Thr-Glu-Pro-,
-Ser-Arg-Leu-Glu-cyclo(hCys-Glu-Leu-DhCys)-Arg-Arg-Leu-Thr-Glu-Pro-,
-Ser-Arg-Leu-Glu-cyclo(DhCys-Glu-Leu-DhCys)-Arg-Arg-Leu-Thr-Glu-Pro-,
-Ser-Arg-Leu-Glu-cyclo(Cys-Glu-Leu-DhCys)-Arg-Arg-Leu-Thr-Glu-Pro-,
-Ser-Arg-Leu-Glu-cyclo(DCys-Glu-Leu-DhCys)-Arg-Arg-Leu-Thr-Glu-Pro-,
-Ser-Arg-Leu-Glu-cyclo(DhCys-Glu-Leu-Cys)-Arg-Arg-Leu-Thr-Glu-Pro-,
-Ser-Arg-Leu-Glu-cyclo(DhCys-Glu-Leu-DCys)-Arg-Arg-Leu-Thr-Glu-Pro-,
-Pro-Ser-Arg-Leu-Glu-cyclo(DCys-Glu-Leu-DCys)-Arg-Arg-Leu-Thr-Glu-Pro-,
-Pro-Ser-Arg-Leu-Glu-cyclo(DCys-Glu-Leu-Cys)-Arg-Arg-Leu-Thr-Glu-Pro-,
-Pro-Ser-Arg-Leu-Glu-cyclo(hCys-Glu-Leu-Cys)-Arg-Arg-Leu-Thr-Glu-Pro-,
-Pro-Ser-Arg-Leu-Glu-cyclo(hCys-Glu-Leu-hCys)-Arg-Arg-Leu-Thr-Glu-Pro-,
-Pro-Ser-Arg-Leu-Glu-cyclo(Cys-Glu-Leu-hCys)-Arg-Arg-Leu-Thr-Glu-Pro-,
-Pro-Ser-Arg-Leu-Glu-cyclo(hCys-Glu-Leu-DCys)-Arg-Arg-Leu-Thr-Glu-Pro-,
-Pro-Ser-Arg-Leu-Glu-cyclo(DCys-Glu-Leu-hCys)-Arg-Arg-Leu-Thr-Glu-Pro-,
-Pro-Ser-Arg-Leu-Glu-cyclo(hCys-Glu-Leu-DhCys)-Arg-Arg-Leu-Thr-Glu-Pro-,
-Pro-Ser-Arg-Leu-Glu-cyclo(DhCys-Glu-Leu-DhCys)-Arg-Arg-Leu-Thr-Glu-Pro-,
-Pro-Ser-Arg-Leu-Glu-cyclo(Cys-Glu-Leu-DhCys)-Arg-Arg-Leu-Thr-Glu-Pro-,
-Pro-Ser-Arg-Leu-Glu-cyclo(DCys-Glu-Leu-DhCys)-Arg-Arg-Leu-Thr-Glu-Pro-,
-Pro-Ser-Arg-Leu-Glu-cyclo(DhCys-Glu-Leu-Cys)-Arg-Arg-Leu-Thr-Glu-Pro-,
-Pro-Ser-Arg-Leu-Glu-cyclo(DhCys-Glu-Leu-DCys)-Arg-Arg-Leu-Thr-Glu-Pro-,
-Ser-Arg-Leu-Glu-Glu-Glu-Leu-Arg-Arg-cyclo(Cys-Leu-Thr-DCys)-Pro-,
-Ser-Arg-Leu-Glu-Glu-Glu-Leu-Arg-Arg-cyclo(DCys-Leu-Thr-Cys)-Pro-,
-Ser-Arg-Leu-Glu-Glu-Glu-Leu-Arg-Arg-cyclo(DCys-Leu-Thr-DCys)-Pro-,
-Ser-Arg-Leu-Glu-Glu-Glu-Leu-Arg-Arg-cyclo(hCys-Leu-Thr-Cys)-Pro-,
-Ser-Arg-Leu-Glu-Glu-Glu-Leu-Arg-Arg-cyclo(hCys-Leu-Thr-DCys)-Pro-,
-Ser-Arg-Leu-Glu-Glu-Glu-Leu-Arg-Arg-cyclo(hCys-Leu-Thr-hCys)-Pro-,
-Ser-Arg-Leu-Glu-Glu-Glu-Leu-Arg-Arg-cyclo(Cys-Leu-Thr-hCys)-Pro-,
-Ser-Arg-Leu-Glu-Glu-Glu-Leu-Arg-Arg-cyclo(DCys-Leu-Thr-hCys)-Pro-,
-Ser-Arg-Leu-Glu-Glu-Glu-Leu-Arg-Arg-cyclo(Cys-Leu-Thr-hCys)-Pro-,
-Ser-Arg-Leu-Glu-Glu-Glu-Leu-Arg-Arg-cyclo(DhCys-Leu-Thr-Cys)-Pro-,
-Ser-Arg-Leu-Glu-Glu-Glu-Leu-Arg-Arg-cyclo(DhCys-Leu-Thr-DCys)-Pro-,
-Ser-Arg-Leu-Glu-Glu-Glu-Leu-Arg-Arg-cyclo(DhCys-Leu-Thr-hCys)-Pro-,
-Ser-Arg-Leu-Glu-Glu-Glu-Leu-Arg-Arg-cyclo(Cys-Leu-Thr-DhCys)-Pro-,
-Ser-Arg-Leu-Glu-Glu-Glu-Leu-Arg-Arg-cyclo(DCys-Leu-Thr-DhCys)-Pro-,
-Ser-Arg-Leu-Glu-Glu-Glu-Leu-Arg-Arg-cyclo(Cys-Leu-Thr-DhCys)-Pro-,
-Ser-Arg-Leu-Glu-Glu-Glu-Leu-Arg-Arg-cyclo(Pen-Leu-Thr-Pen)-Pro-,
-Ser-Arg-Leu-Glu-Glu-Glu-Leu-Arg-Arg-cyclo(Pen-Leu-Thr-DPen)-Pro-,
-Ser-Arg-Leu-Glu-Glu-Glu-Leu-Arg-Arg-cyclo(DPen-Leu-Thr-Pen)-Pro-,
-Ser-Arg-Leu-Glu-Glu-Glu-Leu-Arg-Arg-cyclo(DPen-Leu-Thr-DPen)-Pro-,
-Ser-Arg-Leu-Glu-Glu-Glu-Leu-Arg-cyclo(DCys-Arg-Leu-Cys)-Glu-Pro-,
-Ser-Arg-Leu-Glu-Glu-Glu-Leu-Arg-cyclo(DCys-Arg-Leu-DCys)-Glu-Pro-,
-Ser-Arg-Leu-Glu-Glu-Glu-Leu-Arg-cyclo(Cys-Arg-Leu-DCys)-Glu-Pro-,
-Ser-Arg-Leu-Glu-Glu-Glu-Leu-Arg-cyclo(hCys-Arg-Leu-Cys)-Glu-Pro-,
-Ser-Arg-Leu-Glu-Glu-Glu-Leu-Arg-cyclo(hCys-Arg-Leu-DCys)-Glu-Pro-,
-Ser-Arg-Leu-Glu-Glu-Glu-Leu-Arg-cyclo(Cys-Arg-Leu-hCys)-Glu-Pro-,
-Ser-Arg-Leu-Glu-Glu-Glu-Leu-Arg-cyclo(hCys-Arg-Leu-hCys)-Glu-Pro-,
-Ser-Arg-Leu-Glu-Glu-Glu-Leu-Arg-cyclo(hCys-Arg-Leu-DhCys)-Glu-Pro-,
-Ser-Arg-Leu-Glu-Glu-Glu-Leu-Arg-cyclo(DhCys-Arg-Leu-hCys)-Glu-Pro-,
-Pro-Ser-Arg-Leu-Glu-Glu-Glu-Leu-Arg-Arg-cyclo(Cys-Leu-Thr-DCys)-Pro-,
-Pro-Ser-Arg-Leu-Glu-Glu-Glu-Leu-Arg-Arg-cyclo(DCys-Leu-Thr-Cys)-Pro-,
-Pro-Ser-Arg-Leu-Glu-Glu-Glu-Leu-Arg-Arg-cyclo(DCys-Leu-Thr-DCys)-Pro-,
-Pro-Ser-Arg-Leu-Glu-Glu-Glu-Leu-Arg-Arg-cyclo(hCys-Leu-Thr-Cys)-Pro-,
-Pro-Ser-Arg-Leu-Glu-Glu-Glu-Leu-Arg-Arg-cyclo(hCys-Leu-Thr-DCys)-Pro-,
-Pro-Ser-Arg-Leu-Glu-Glu-Glu-Leu-Arg-Arg-cyclo(hCys-Leu-Thr-hCys)-Pro-,
-Pro-Ser-Arg-Leu-Glu-Glu-Glu-Leu-Arg-Arg-cyclo(Cys-Leu-Thr-hCys)-Pro-,
-Pro-Ser-Arg-Leu-Glu-Glu-Glu-Leu-Arg-Arg-cyclo(DCys-Leu-Thr-hCys)-Pro-,
-Pro-Ser-Arg-Leu-Glu-Glu-Glu-Leu-Arg-Arg-cyclo(Cys-Leu-Thr-hCys)-Pro-,
-Pro-Ser-Arg-Leu-Glu-Glu-Glu-Leu-Arg-Arg-cyclo(DhCys-Leu-Thr-Cys)-Pro-,
-Pro-Ser-Arg-Leu-Glu-Glu-Glu-Leu-Arg-Arg-cyclo(DhCys-Leu-Thr-DCys)-Pro-,
-Pro-Ser-Arg-Leu-Glu-Glu-Glu-Leu-Arg-Arg-cyclo(DhCys-Leu-Thr-hCys)-Pro-,
-Pro-Ser-Arg-Leu-Glu-Glu-Glu-Leu-Arg-Arg-cyclo(Cys-Leu-Thr-DhCys)-Pro-,
-Pro-Ser-Arg-Leu-Glu-Glu-Glu-Leu-Arg-Arg-cyclo(DCys-Leu-Thr-DhCys)-Pro-,
-Pro-Ser-Arg-Leu-Glu-Glu-Glu-Leu-Arg-Arg-cyclo(Cys-Leu-Thr-DhCys)-Pro-,
-Pro-Ser-Arg-Leu-Glu-Glu-Glu-Leu-Arg-Arg-cyclo(Pen-Leu-Thr-Pen)-Pro-,
-Pro-Ser-Arg-Leu-Glu-Glu-Glu-Leu-Arg-Arg-cyclo(Pen-Leu-Thr-DPen)-Pro-,
-Pro-Ser-Arg-Leu-Glu-Glu-Glu-Leu-Arg-Arg-cyclo(DPen-Leu-Thr-Pen)-Pro-,
-Pro-Ser-Arg-Leu-Glu-Glu-Glu-Leu-Arg-Arg-cyclo(DPen-Leu-Thr-DPen)-Pro-,
-Pro-Ser-Arg-Leu-Glu-Glu-Glu-Leu-Arg-cyclo(DCys-Arg-Leu-Cys)-Glu-Pro-,
-Pro-Ser-Arg-Leu-Glu-Glu-Glu-Leu-Arg-cyclo(DCys-Arg-Leu-DCys)-Glu-Pro-,
-Pro-Ser-Arg-Leu-Glu-Glu-Glu-Leu-Arg-cyclo(Cys-Arg-Leu-DCys)-Glu-Pro-,
-Pro-Ser-Arg-Leu-Glu-Glu-Glu-Leu-Arg-cyclo(hCys-Arg-Leu-Cys)-Glu-Pro-,
-Pro-Ser-Arg-Leu-Glu-Glu-Glu-Leu-Arg-cyclo(hCys-Arg-Leu-DCys)-Glu-Pro-,
-Pro-Ser-Arg-Leu-Glu-Glu-Glu-Leu-Arg-cyclo(Cys-Arg-Leu-hCys)-Glu-Pro-,
-Pro-Ser-Arg-Leu-Glu-Glu-Glu-Leu-Arg-cyclo(hCys-Arg-Leu-hCys)-Glu-Pro-,
-Pro-Ser-Arg-Leu-Glu-Glu-Glu-Leu-Arg-cyclo(hCys-Arg-Leu-DhCys)-Glu-Pro-,
-Pro-Ser-Arg-Leu-Glu-Glu-Glu-Leu-Arg-cyclo(DhCys-Arg-Leu-hCys)-Glu-Pro-,
-Ser-Arg-Leu-Glu-Glu-Glu-Leu-Arg-cyclo(Lys-Arg-Leu-Asp)-Glu-Pro-,
-Ser-Arg-Leu-Glu-Glu-Glu-Leu-Arg-cyclo(Asp-Arg-Leu-Lys)-Glu-Pro-,
-Ser-Arg-Leu-Glu-Glu-Glu-Leu-Arg-cyclo(Lys-Arg-Leu-Glu)-Glu-Pro-,
-Ser-Arg-Leu-Glu-Glu-Glu-Leu-Arg-cyclo(Glu-Arg-Leu-Lys)-Glu-Pro-,
-Pro-Ser-Arg-Leu-Glu-Glu-Glu-Leu-Arg-cyclo(Lys-Arg-Leu-Asp)-Glu-Pro-,
-Pro-Ser-Arg-Leu-Glu-Glu-Glu-Leu-Arg-cyclo(Asp-Arg-Leu-Lys)-Glu-Pro-,
-Pro-Ser-Arg-Leu-Glu-Glu-Glu-Leu-Arg-cyclo(Lys-Arg-Leu-Glu)-Glu-Pro-,
-Pro-Ser-Arg-Leu-Glu-Glu-Glu-Leu-Arg-cyclo(Glu-Arg-Leu-Lys)-Glu-Pro-,
-Pro-Ser-Arg-Leu-Glu-Glu-Glu-Leu-Arg-cyclo(Cys-Arg-Leu-Thr-Cys)-Pro-,
-Pro-Ser-Arg-Leu-Glu-cyclo(Cys-Glu-Leu-Arg-Cys)-Arg-Leu-Thr-Glu-Pro-,
-Pro-Ser-Arg-Leu-Glu-Glu-Glu-Leu-Arg-Arg-cyclo(Cys-Leu-Thr-Cys)-Pro-,
-Pro-Ser-Arg-Leu-Glu-Glu-cyclo(Cys-Leu-Arg-Arg-Cys)-Leu-Thr-Glu-Pro-,
-Ser-Arg-Leu-Glu-cyclo(Lys-Glu-Leu-Arg-Glu)-Arg-Leu-Thr-Glu-,
and
-Ser-Arg-Leu-cyclo(Glu-Glu-Glu-Leu-Lys)-Arg-Arg-Leu-Thr-Glu -.

In some embodiments, the cyclic peptide is attached to a 3-mercaptopropionyl moiety through an α-amine moiety of the leftmost amino acid in the cyclic peptide. In some embodiments, the rightmost am no acid in the cyclic peptide comprises an amide.

In some embodiments, the cyclized amino acid sequence is one selected from the group consisting of

-Ser-Arg-Leu-Glu-cyclo(Glu-Glu-Leu-Arg-Lys)-Arg-Leu-Thr-Glu-,
-Ser-Arg-Leu-Glu-cyclo(Asp-Glu-Leu-Arg-Lys)-Arg-Leu-Thr-Glu-,
Ser-Arg-Leu-Glu-cyclo(Glu-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Glu)-,
-Ser-Arg-Leu-Glu-cyclo(Cys-Glu-Leu-Arg-Cys)-Arg-Leu-Thr-Glu-,
-Ser-Arg-Leu-Glu-cyclo(Asp-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Asp)-,
-Ser-Arg-Leu-Glu-cyclo(Glu-Glu-Leu-Arg-DLys)-Arg-Leu-Thr-Glu-
-Pro-Ser-Arg-Leu-Glu-cyclo(Glu-Glu-Leu-Arg-Lys)-Arg-Leu-Thr-Glu-,
-Pro-Ser-Arg-Leu-Glu-cyclo(DGlu-Glu-Leu-Arg-Lys)-Arg-Leu-Thr-Glu-
-Pro-Ser-Arg-Leu-Glu-cyclo(Glu-Glu-Leu-Arg-DLys)-Arg-Leu-Thr-Glu-
-Pro-Ser-Arg-Leu-Glu-cyclo(Lys-Glu-Leu-Arg-Glu)-Arg-Leu-Thr-Glu-
-Pro-Ser-Arg-Leu-cyclo(Glu-Glu-Glu-Leu-Lys)-Arg-Arg-Leu-Thr-Glu-,
-Pro-Ser-Arg-Leu-Glu-cyclo(Cys-Glu-Leu-DCys)-Arg-Arg-Leu-Thr-Glu-,
-Pro-Ser-Arg-Leu-Glu-Glu-Glu-Leu-Arg-cyclo(Cys-Arg-Leu-Thr-Cys)-,
-Pro-Ser-Arg-Leu-Glu-cyclo(Cys-Glu-Leu-Arg-Cys)-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-cyclo(Cys-Leu-Arg-Arg-Cys)-Leu-Thr-Glu-,
-Ser-Arg-Leu-Glu-Glu-Glu-Leu-Arg-cyclo(Cys-Arg-Leu-Thr-Cys)-,
-Ser-Arg-Leu-Glu-cyclo(Cys-Glu-Leu-Arg-Cys)-Arg-Leu-Thr-Glu-,
-Ser-Arg-Leu-Glu-cyclo(Cys-Glu-Leu-Cys)-Arg-Arg-Leu-Thr-Glu-,
-Ser-Arg-Leu-Glu-Glu-Glu-Leu-Arg-Arg-cyclo(Cys-Leu-Thr-Cys)-,
-Ser-Arg-Leu-Glu-Glu-cyclo(Cys-Leu-Arg-Arg-Cys)-Leu-Thr-Glu-,
-Ser-Arg-Leu-Glu-cyclo(Lys-Glu-Leu-Arg-Glu)-Arg-Leu-Thr-Glu-,
and
-Ser-Arg-Leu-cyclo(Glu-Glu-Glu-Leu-Lys)-Arg-Arg-Leu-Thr-Glu -.

In some embodiments, the cyclized amino acid sequence is -Ser-Arg-Leu-Glu-cyclo(Glu-Glu-Leu-Arg-Lys)-Arg-Leu-Thr-Glu-. In some other embodiments, the cyclized amino acid sequence is -Ser-Arg-Leu-Glu-cycl(Asp-Glu-Leu-Arg-Lys)-Arg-Leu-Thr-Glu-. In yet some other embodiments, the cyclized amino acid sequence is -Ser-Arg-Leu-Glu-cyclo(Glu-Glu-Leu-Lys)-Arg-Arg-Leu-Thr-Glu-. In still some other embodiments, the cyclized amino acid sequence is -Ser-Arg-Leu-Glu-Glu-Glu-Leu-Arg-cyclo(Lys-Arg-Leu-Thr-Glu)-.

The cyclic peptides may have different cyclic bridging moieties forming the ring structure. Preferably, chemically stable bridging moieties are included in the ring structure such as, for example, an amide group, a lactone group, an ether group, a thioether group, a disulfide group, an alkylene group, an alkenyl group, or a 1,2,3-triazole. The following are examples illustrating the variability of bridging moieties in a peptide:

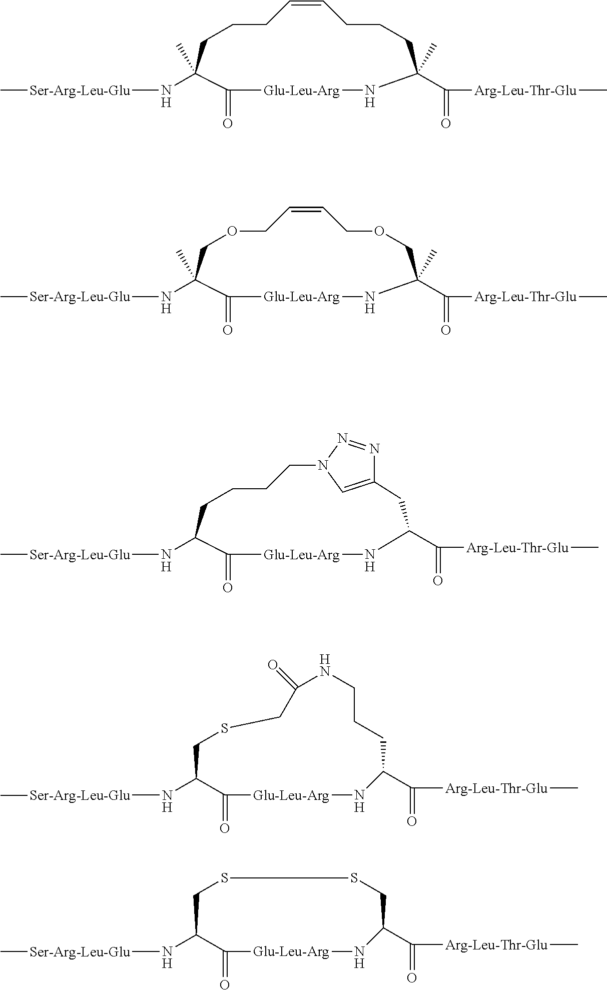

In some embodiments, a peptide comprising a cyclized amino acid sequence disclosed herein binds to a compound comprising a moiety binding to a peptide comprising a cyclized amino acid sequence disclosed herein. In some embodiments, the moiety binding to the peptide comprising a cyclized amino acid sequence binds to the peptide by binding to the cyclized amino acid sequence. In some embodiments, the moiety binding to the peptide comprising a cyclized amino acid sequence comprises an antibody or antibody fragment, e.g., an antibody disclosed herein such as a camelid VHH domain disclosed herein.

It has been found that the straight-chain ALFA^{SM} peptide is subject to decreased stability in serum and plasma due to the enzymatic cleavage at the peptide bond between Arg-Leu residues as shown below.

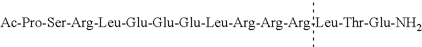

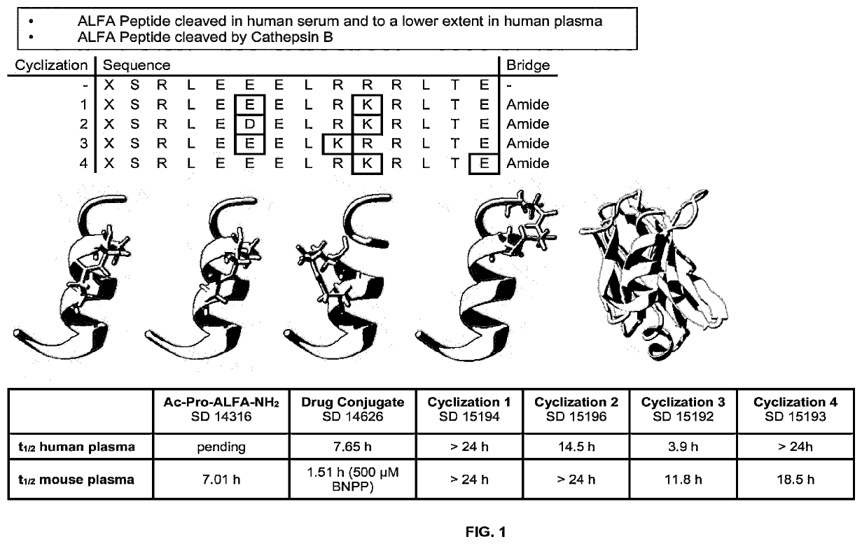

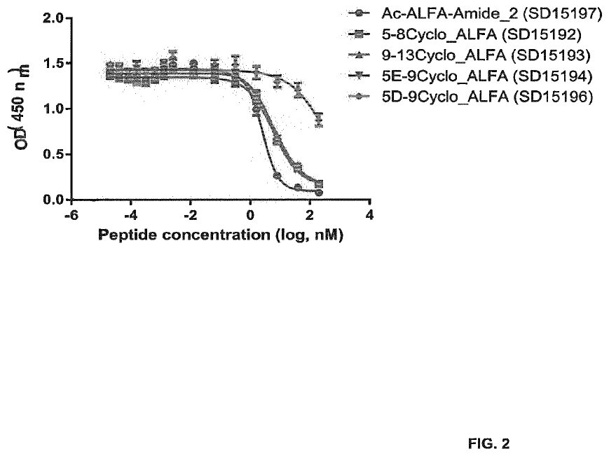

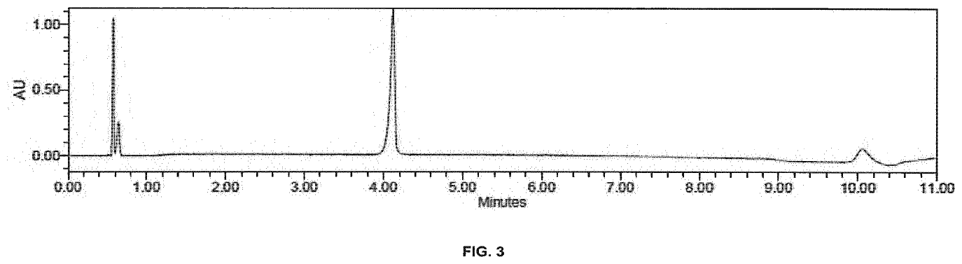

Without intending to be bound by any particular theory, it is believed that the cyclic ring can protect the peptide bonds inside or near it from enzymatic cleavage. On addition, the cyclic structures can enhance α-helicity and therefore retain the binding affinity to the anti-ALFA VHH that recognizes the α-helical structure of the peptide. For example, referring to FIG. 1, embodiments of cyclic ALFA peptides shown demonstrated longer half-lives in human and mouse plasma in in vitro assays. Among the cyclic ALFA peptides, H-Ser-Arg-Leu-Glu-cyclo(Glu-Glu-Leu-Arg-Lys)-Arg-Leu-Thr-Glu-OH peptide, which is “cyclization 1” in FIG. 1 and “Ac_5E-9Cyclo_ALFA” in Table 1, has a half-life longer than 24 h in both human and mouse plasma (FIG. 1). Importantly, this cyclic peptide retains the binding affinity to the anti-ALFA VHH with an EC50 of 6.0 nM compared to 2.6 nM of the parent linear ALFA peptide (FIG. 2 and Table 1).

TABLE 1

| | Ac-ALFA-Amide_2 | 5-8Cyclo_ALFA | 9-13Cyclo_ALFA | 5E-9Cyclo_ALFA | 5D-9Cyclo_ALFA |
|---|---|---|---|---|---|
| | (SD15197) | (SD15192) | (SD15193) | (SD15194) | (SD15196) |
| log(agonist) vs. | | | | | |
| response-- | | | | | |
| Variable slope | | | | | |
| Best-fit values | | | | | |
| Bottom | 0.09699 | 0.1519 | 0.1452 | 0.1593 | 0.01671 |
| Top | 1.387 | 1.350 | 1.440 | 1.428 | 1.426 |
| LogEC50 | 0.4145 | 0.7765 | 0.7818 | 0.7763 | 2.576 |
| HillSlope | −1.670 | −0.9841 | −0.8859 | −0.9926 | −0.7157 |
| EC50 | 2.597 | 5.977 | 6.051 | 5.975 | 377.1 |
| Span | 1.290 | 1.198 | 1.295 | 1.269 | 1.410 |

The peptide comprising a cyclized amino acid sequence disclosed herein may be a component of a larger unit, e.g., a compound or complex of compounds.

In some embodiments, such larger unit may be a compound comprising one or more peptides comprising a cyclized amino acid sequence disclosed herein and (i) one or more peptidic moieties other than the peptide comprising a cyclized amino acid sequence disclosed herein, (ii) one or more non-peptidic moieties, or (iii) a combination of (i) and (ii). In some embodiments, the one or more peptides comprising a cyclized amino acid sequence disclosed herein may be linked to the one or more peptidic moieties other than the peptide comprising a cyclized amino acid sequence disclosed herein and/or the one or more non-peptidic moieties either by direct fusion or through a linker. A “linker” as used herein joins together two or more subunits, e.g., of a fusion protein. The linkage can be covalent. In some embodiments, a covalent linkage is via a peptide bond, such as a peptide bond between amino acids. In some embodiments, a linker is a peptide linker. In some embodiments, a linker comprises one or more amino acids, such as 1, 2, 3, 4, 5, 6, 7, 8, 9, 10, 11, 12, 13, 14, 15, 16, 17, 18, 19, 20 or more amino acids. Peptide linkers include glycine-serine (GS) linkers, glycosylated GS linkers, and proline-alanine-serine polymer (PAS) linkers.

In some embodiments, such larger unit may be a complex of two or more compounds, wherein at least one compound comprises one or more peptides comprising a cyclized amino acid sequence disclosed herein.

In some embodiments, a peptide comprising a cyclized amino acid sequence disclosed herein may be a component a fusion protein. In some embodiments, a fusion protein is a recombinant protein.

As used herein, the terms “linked”, “fused”, or “fusion” are used interchangeably.

These terms refer to the joining together of two or more elements or components or domains.

The term “fusion protein” as used herein refers to a polypeptide or protein comprising two or more subunits. Preferably, the fusion protein is a translational fusion between the two or more subunits. The translational fusion may be generated by genetically engineering the coding nucleotide sequence for one subunit in a reading frame with the coding nucleotide sequence of a further subunit. Subunits may be interspersed by a linker.

In the fusion protein disclosed herein, the peptide, i.e. the epitope tag, disclosed herein may be located at any position of the fusion protein. The peptide may be fused to the N-terminus or the C-terminus of the polypeptide to which it is fused. Alternatively, the peptide may be fused internally to the polypeptide at a position between the N-terminus and the C-terminus of the polypeptide. As an illustrative example, the peptide may be fused in between two domains of the polypeptide.

The term “recombinant” in the context of the present disclosure means “made through genetic engineering”. In some embodiments, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

In some embodiments, a peptide comprising a cyclized amino acid sequence disclosed herein may be a component a functionalized stealth lipid such as, for example, those disclosed in U.S. provisional patent application Ser. No. 63/305,905, filed on Feb. 2, 2022, the disclosure of which is incorporated herein by reference. Stealth lipids are lipids that increase the length of time for which the nanoparticles can exist in vivo (e.g., in the blood).

The terms “lipid” and “lipid-like material” are broadly defined herein as a molecule that comprises one or more hydrophobic moieties or groups and optionally one or more hydrophilic groups attached either directly or indirectly to a lipid portion. As used herein, the term “lipid” is to be construed to cover both lipids and lipid-like materials unless otherwise indicated herein or clearly contradicted by context.

Molecules comprising hydrophobic moieties and hydrophilic moieties are also frequently referred to as amphiphiles. In an aqueous environment, the amphiphilic nature allows such molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge and be a zwitterion or inner salt. For purposes of the disclosure, the amphiphilic compound can be, but is not limited to, one or a plurality of natural or non-natural lipids and lipid-like compounds.

Specific examples of amphiphilic compounds that may be included in an amphiphilic layer include, but are not limited to, phospholipids, aminolipids and sphingolipids. In certain embodiments, the amphiphilic compound is a lipid. The term “lipid” refers to a group of organic compounds that are characterized by being insoluble in water, but soluble in many organic solvents. Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

Self-assembled amphiphiles as disclosed are referred to herein as a “lipid nanoparticle.” Thus, “lipid nanoparticle” refers to a particle that comprises a plurality of (i.e., more than one) lipid molecules physically associated with each other by intermolecular forces. The nanoparticles may be, e.g., microspheres (including unilamellar and multilamellar vesicles, e.g., “liposomes”—lamella phase lipid bilayers that, in some embodiments are substantially spherical (and may include non-spherical morphology), and, in more particular embodiments can comprise a core that is aqueous or non-aqueous e.g., a dispersed phase in an emulsion, micelles or an internal phase in a suspension.

In some embodiments, a complex comprising a peptide comprising a cyclized amino acid sequence disclosed herein comprises compound comprising a peptide comprising a cyclized amino acid sequence disclosed herein and a compound comprising a moiety binding to the peptide comprising a cyclized amino acid sequence. In some embodiments, the moiety binding to the peptide comprising a cyclized amino acid sequence comprises an antibody or antibody fragment. In some embodiments the antibody is a monovalent antibody. In some embodiments the antibody is a single domain antibody. In some embodiments, the antibody comprises or consists of a VHH domain, e.g., a camelid VHH domain.

In some embodiments, the moiety binding to the peptide comprising a cyclized amino acid sequence comprises a single domain antibody, e.g., a camelid VHH domain comprising the CDR1 sequence VTX₁SALNAMAMG, wherein X₁ is I or V, the CDR2 sequence AVSX₂RGNAM, wherein X₂ is E, H, N, D, or S, and the CDR3 sequence LEDRVDSFHDY.

In some embodiments, the moiety binding to the peptide comprising a cyclized amino acid sequence comprises a single domain antibody, e.g., a camelid VHH domain comprising the CDR1 sequence GVTX₁SALNAMAMG wherein X₁ is I or V, the CDR2 sequence AVSX₂RGNAM, wherein X₂ is E, H, N D, or S, and the CDR3 sequence LEDRVDSFHDY.

In some embodiments, the moiety binding to the peptide comprising a cyclized amino acid sequence comprises a single domain antibody, e.g., a camelid VHH domain comprising the CDR1 sequence VTISALNAMAMG, the CDR2 sequence AVSERGNAM, and the CDR3 sequence LEDRVDSFHDY.

In some embodiments, the moiety binding to the peptide comprising a cyclized amino acid sequence comprises a single domain antibody, e.g., a camelid VHH domain comprising the CDR1 sequence GVTISALNAMAMG, the CDR2 sequence AVSERGNAM, and the CDR3 sequence LEDRVDSFHDY.

In some embodiments, the moiety binding to the peptide comprising a cyclized amino acid sequence comprises a single domain antibody, e.g., a camelid VHH domain comprising the amino acid sequence EVQLQESGGGLVQPGGSLRLSCTASGVTISALNAMAMGWYRQAPGERRVMVAAVSERGN AMYRESVQGRFTVTRDFTNKMVSLQMDNLKPEDTAVYYCHVLEDRVDSFHDYWGQGTQV TVSS, an amino acid sequence having at least 99%, 98%, 97%, 96%, 95%, 90%, 85%, or 80% identity to said amino acid sequence, or a fragment of said amino acid sequence or the amino acid sequence having at least 99%, 98%, 97%, 96%, 95%, 90%, 85%, or 80% identity to said amino acid sequence. In some embodiments, the amino acid sequence comprises CDR1, CDR2 and CDR3 sequences as described above.

The term “bind” or “binding” relates to the non-covalent interaction with a target. In some embodiments, the term “bind” or “binding” relates to a specific binding. By the term “specific binding” or “specifically binds”, as used herein, is meant a molecule such as an antibody which recognizes a specific target molecule, but does not substantially recognize or bind other molecules in a sample or in a subject. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binds”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “binding” or “capable of binding” typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less, when determined using Bio-Layer Interferometry (BLI), or, for instance, when determined using surface plasmon resonance (SPR) technology in a BIAcore 3000 instrument. In some embodiments, a binding moiety or agent binds to a predetermined target with an affinity corresponding to a K_{D} that is at least ten-fold lower, such as at least 100-fold lower, for instance at least 1,000-fold lower, such as at least 10,000 fold lower, for instance at least 100,000-fold lower than its affinity for binding to a non-specific target (e.g., BSA, casein).

The term “k_{d}” (sec⁻¹), as used herein, refers to the dissociation rate constant of a particular interaction, e.g., antibody-antigen interaction. Said value is also referred to as the k_{off} value.

The term “K_{D}” (M), as used herein, refers to the dissociation equilibrium constant of a particular interaction, e.g., antibody-antigen interaction.

Disclosed herein are binding moieties and binding agents binding to a peptide comprising a cyclized amino acid sequence disclosed herein. Such binding moieties and binding agents may form complexes with the peptide.

The term “binding agent” as used herein refers to any agent capable of binding to desired antigens. In certain embodiments, the binding agent is or comprises an antibody, antibody fragment, or any other binding protein, or any combination thereof.

In some embodiments, binding agents disclosed herein comprise bispecific or multispecific binding agents such as bispecific antibodies comprising a first and a second binding domain, wherein the first binding domain is capable of binding to a peptide comprising a cyclized amino acid sequence disclosed herein and the second binding domain is capable of binding to a different target.

The term “binding moiety” as used herein refers to any moiety, group or domain capable of binding to desired antigens. In certain embodiments, the binding moiety is or comprises an antibody, antibody fragment, or any other binding protein, or any combination thereof.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (abbreviated herein as C_{H} or CH). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as V_{L} or VL) and a light chain constant region (abbreviated herein as C_{L} or CL). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, reference to amino acid positions in the constant regions in the present disclosure is according to the EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242). In general, CDRs described herein are Kabat defined.

The term “antibody” (Ab) as used herein refers to an immunoglobulin molecule, a fragment of an immunoglobulin molecule, or a derivative of either thereof, which has the ability to bind, preferably specifically bind to an antigen or epitope. The variable regions of the heavy and light chains of the immunoglobulin molecule contain a binding domain that interacts with an antigen or epitope.

The term “antigen-binding region”, “binding region” or “binding domain”, as used herein, refers to the region or domain which interacts with the antigen and typically comprises both a VH region and a VL region.

The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody as used herein, unless otherwise stated or clearly contradicted by context, includes fragments of an antibody that are antigen-binding fragments, i.e., retain the ability to specifically bind to the antigen, and antibody derivatives, i.e., constructs that are derived from an antibody. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol. 2003 November; 21(11):484-90); (vi) camelid or Nanobody molecules (Revets et al; Expert Opin Biol Ther. 2005 January; 5(1):111-24) and (vii) an isolated complementarity determining region (CDR).

Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al, Science 242, 423-426 (1988) and Huston et at, PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present disclosure, exhibiting different biological properties and utility. These and other useful antibody fragments in the context of the present disclosure, as well as bispecific formats of such fragments, are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

The phrase “single chain Fv” or “scFv” refers to an antibody in which the variable domains of the heavy chain and of the light chain (VH and VL) of a traditional two chain antibody have been joined to form one chain. Optionally, a linker (usually a peptide) is inserted between the two chains to allow for proper folding and creation of an active binding site.

A single-domain antibody, also known as a nanobody, is an antibody fragment consisting of a single monomeric variable antibody domain. In some embodiments, a single-domain antibody is a variable domain (V_{H}) of a heavy-chain antibody. These are called VHH fragments. Like a whole antibody, a single-domain antibody is able to bind selectively to a specific antigen. The first single-domain antibodies were engineered from heavy-chain antibodies found in camelids. Cartilaginous fishes also have heavy-chain antibodies (IgNAR, ‘immunoglobulin new antigen receptor’), from which single-domain antibodies called VNAR fragments can be obtained. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes.

An antibody can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody may be a sequence variant of a naturally-occurring IgG1 antibody, including variations in the constant regions.

In various embodiments, an antibody is an IgG1 antibody, more particularly an IgG1, kappa or IgG1, lambda isotype (i.e. IgG1, κ, λ), an IgG2a antibody (e.g. IgG2a, κ, λ), an IgG2b antibody (e.g. IgG2b, κ, λ), an IgG3 antibody (e.g. IgG3, κ, λ) or an IgG4 antibody (e.g. IgG4, κ, λ).

The term “monoclonal antibody” as used herein refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

The term “chimeric antibody” as used herein, refers to an antibody wherein the variable region is derived from a non-human species (e.g. derived from rodents) and the constant region is derived from a different species, such as human. Chimeric monoclonal antibodies for therapeutic applications are developed to reduce antibody immunogenicity. The terms “variable region” or “variable domain” as used in the context of chimeric antibodies, refer to a region which comprises the CDRs and framework regions of both the heavy and light chains of the immunoglobulin. Chimeric antibodies may be generated by using standard DNA techniques as described in Sambrook et al, 1989, Molecular Cloning: A laboratory Manual, New York: Cold Spring Harbor Laboratory Press, Ch. 15. The chimeric antibody may be a genetically or an enzymatically engineered recombinant antibody. It is within the knowledge of the skilled person to generate a chimeric antibody, and thus, generation of the chimeric antibody may be performed by other methods than described herein.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid backmutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “human antibody” as used herein, refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse or rat, have been grafted onto human framework sequences. Human monoclonal antibodies can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known. Human monoclonal antibodies can thus e.g. be generated using transgenic or transchromosomal mice or rats carrying parts of the human immune system rather than the mouse or rat system. Accordingly, in some embodiments, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogeneous animal antibody machinery, see e.g. Mendez et al. 1997 Nat Genet. 15 (2): 146-56.

When used herein, unless contradicted by context, the term “Fab-arm”, “binding arm” or “arm” includes one heavy chain-light chain pair and is used interchangeably with “half-molecule” herein.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

When used herein, unless contradicted by context, the term “Fc region” refers to an antibody region consisting of the two Fc sequences of the heavy chains of an immunoglobulin, wherein said Fc sequences comprise at least a hinge region, a CH2 domain, and a CH3 domain.

The present disclosure also envisions antibodies comprising functional variants of the VL regions, VH regions, or one or more CDRs of the antibodies described herein. A functional variant of a VL, VH, or CDR used in the context of an antibody still allows the antibody to retain at least a substantial proportion (at least about 50%, 60%, 70%, 80%, 90%, 95% or more) of the affinity and/or the specificity/selectivity of the “reference” or “parent” antibody and in some cases, such an antibody may be associated with greater affinity, selectivity and/or specificity than the parent antibody.

Such functional variants typically retain significant sequence identity to the parent antibody.

Exemplary variants include those which differ from VH and/or VL and/or CDR regions of the parent antibody sequences mainly by conservative substitutions; for instance, up to 10, such as 9, 8, 7, 6, 5, 4, 3, 2 or 1 of the substitutions in the variant are conservative amino acid residue replacements.

Functional variants of antibody sequences described herein such as VL regions, or VH regions, or antibody sequences having a certain degree of homology or identity to antibody sequences described herein such as VL regions, or VH regions preferably comprise modifications or variations in the non-CDR sequences, while the CDR sequences preferably remain unchanged.

The term “specificity” as used herein is intended to have the following meaning unless contradicted by context. Two antibodies have the “same specificity” if they bind to the same antigen and the same epitope.

An antibody or fragment useful herein may compete with a specific antibody or fragment described herein.

The term “competes” and “competition” may refer to the competition between a first antibody and a second antibody to the same antigen. It is well known to a person skilled in the art how to test for competition of antibodies for binding to a target antigen. An example of such a method is a so-called cross-competition assay, which may e.g. be performed as an ELISA or by flow-cytometry. Alternatively, competition may be determined using biolayer interferometry.

Antibodies which compete for binding to a target antigen may bind different epitopes on the antigen, wherein the epitopes are so close to each other that a first antibody binding to one epitope prevents binding of a second antibody to the other epitope. In other situations, however, two different antibodies may bind the same epitope on the antigen and would compete for binding in a competition binding assay. Such antibodies binding to the same epitope are considered to have the same specificity herein. Thus, in some embodiments, antibodies binding to the same epitope are considered to bind to the same amino acids on the target molecule. That antibodies bind to the same epitope on a target antigen may be determined by standard alanine scanning experiments or antibody-antigen crystallization experiments known to a person skilled in the art. Preferably, antibodies or binding domains binding to different epitopes are not competing with each other for binding to their respective epitopes.

### EXAMPLES

### Example 1: Cyclic ALFA Peptide Synthesis

### Materials and Instruments

Liberty Blue HT24 Synthesizer (OEM Corporation)
CEM Corporation L-Amino Acid: Fmoc-Lys(Mmt)-OH.
Sigma/Millipore L-Amino Acid: Fmoc-Glu(O-2-PhiPr)-OH
Advanced Chemtech Amino Acid: Mpa(Trt)-OH
Matrix Innovations L-Amino Acids: Fmoc-Glu(OtBu)-OH, Fmoc-Leu-OH Fmoc-Arg(Pbf)-OH, Fmoc-Ser(tBu)-OH, Fmoc-Thr(tBu)-OH, Fmoc-Pro-OH.
Advanced Chemtech: Sieber Amide Resin, 100-200 mesh
Global & Semi-Protected Cleavage reagents: TFA (JT Baker W729-07 Baker analyzed reagent), triisoproplysilane (Sigma-Aldrich), Ethanedithiol (Sigma-Aldrich), Thioanisole (Sigma-Aldrich), Diethylether (Fischer Scientific), HPLC water (HoneyWell), Dichloromethane (Sigma-Aldrich), t-butanol (Sigma-Aldrich), acetonitrile (The Lab Depot), Hexane (VWR Chemicals).

Synthesis: Synthesis of 3 replicate intermediate peptide sequences is performed at 0.1 mmol scale using the Liberty Blue HT24 synthesizer. Sieber Amide resins are loaded onto the Liberty HT suspended in 10 mL of 1:1 dichloromethane/dimethylformamide (DCM:DMF) for pre-swelling and resin transfer. The synthesis methods are begun with deprotection of the N-terminal a Fmoc protecting group using 4 mL of 20% piperidine in DMF heated by microwave for 3 min at 60° C. with nitrogen dispensing every three seconds to mix. After draining, the resin is washed three times with 5 mL DMF at five seconds per wash. Then for the coupling reaction 2.5 mL of the next 0.3M amino acid solution (0.5 eq), 1 mL 1M DIC (10 eq), and 0.5 mL 1M Oxyma+0.1M diisopropylethylamine (DIEA) (5 eq) is added to the reaction vessel (RV). The double coupling steps are proceeded with microwave heating for an initial 6 min cycle at 50° C. and an additional 10-minute cycle at 50° C. with nitrogen dispensing every three seconds to mix during both coupling cycles. After the second coupling time completed the resins are drained and 20% piperidine in DMF is added to the RV to start deprotection for the next incoming amino acid. This cycle of Fmoc removal and coupling is repeated for every amino acid sequentially. The Liberty Blue external position #6 is used for addition of Fmoc-Glu(O-2-PhiPr)—OH #7 is used for addition of the Fmoc-Lys(Mmt)-OH amino acid into the peptide sequence as indicated in Table 2.

TABLE 2
List of lactam peptides used; amino acids (E) & (K) in sequence
indicate position for lactam bridge.

| Peptide Batch ID | Peptide Sequence | Theoretical MW |
|---|---|---|
| Cyclic_ALFA_S_Mpa | (Mpa)SRLEEELRKRLTE-NH₂ | 1729.3 |
| Cyclic_ALFA_P_Mpa | (Mpa)PSRLEEELRKRLTE-NH₂ | 1826.4 |

No final deprotection of the peptides is needed due to no presence of Fmoc protection of the Mpa(Trt)-OH amino acid, peptide resins are washed four times with 4 mL DMF followed by transfer from the RV back to the starting HT position and the next peptide in the queue began synthesis.

Combined protected peptide cleavage and orthogonal deprotection of Lysine & Glutamic Acid side chain: After the instrument synthesis run is completed for each peptide, resins are transferred to a 24 mL fritted syringe using DCM and rinsed 3 times with DCM to remove excess DMF. After washing the resins are treated 6 times with 5 mL of 2% trifluoroacetic acid (TFA), 1% triisopropylsilane (TIS) in DCM for roughly 5 minutes each reaction. The flow through solution from each reaction which contained the semi-protected peptides is collected in a separate 50 mL conical tube for each peptide. The semi-protected peptide solutions are then evaporated under a gentle nitrogen (N₂) blanket until the total volume remaining in each tube is less than 5 ml. Once the volume in each conical tube is less than 5 mL the peptides are re-suspended in 20 mL of 1:1 acetonitrile:t-butanol (MeCN:tBu-OH) and sonicated until the solutions are homogenous. The peptide tubes are then frozen at −80° C. and placed on a lyophilizer for drying.

Lactam Cyclization via side chain amide bond formation: Once the semi-protected peptides are dry roughly 5 eq relative to the 0.1 mmol scale of synthesis of (7-Azabenzotriazol-1-yloxy)trispyrrolidinophosphonium hexafluorophosphate (PyAOP) and 1-Hydroxy-7-azabenzotriazole (HOAt) is weighed out and dissolved in 4 mL of DMF. Once the reagents are completely dissolved this solution is added to each of the peptide tubes. After the semi-protected peptide material is dissolved roughly 10 eq relative to the 0.1 mmol scale of synthesis of DIEA is added to each solution and a slight color change where the solution turned a yellow color is observed. The solutions are then left to react for 2-3 hrs at RT.

Precipitation of protected lactam peptides: After 2-3 hrs of reaction time at RT the protected peptides are precipitated by adding roughly 45 mL of chilled HPLC water to each 50 mL conical tube and the tubes are then centrifuged at 1000-1200 rpm for roughly 5 mins. This is repeated 2 times to remove any excess DMF and after the second precipitation the peptide pellets are resuspended in 1:1 (ACN:tBu-OH) frozen at −80° C. and lyophilized overnight. This is done to assess if there is unexpected loss of peptide during the precipitation steps mentioned above.

Global Peptide Cleavage: After the peptides completed drying by lyophilization of roughly 10 mL of cleavage cocktail containing 92.5% (TFA), 2.5% water (H₂O), 2.5% (TIS), and 2.5% thioanisole is added to each 50 mL conical tube and left to shake at RT for 2-3 hours.

Final precipitation: The 10 mL of cleavage cocktail is split into two 50 mL conical tubes at 5 mL each. Roughly 45 mL cold (−20° C.) 1:1 hexane:diethylether is added to each tube. Then the mixture is centrifuged at 3500 rpm for 10 mins. After decanting the ether, another 40 mL cold 1:1 hexane:diethylether is added to further wash the peptide pellets. The container is agitated to break up the peptide pellets and then centrifuged again at 3500 rpm for 10 mins. After decanting the ether a second time, the tubes we placed on their side within a fume hood and allowed to dry. The peptide pellets are then reconstituted in roughly 10 mL of 1:1 acetonitrile/water (ACN:H₂O), frozen at −80° C. and lyophilized.

UPLC-MS analysis and peptide QC sample prep: A small sample of the dried crude peptide powder is transferred to a 1.5 mL Eppendorf tube, and then dissolved with enough 3:1 dimethyl sulfoxide/water (DMSO:H₂O) in order to make an approximate 2 mg/mL peptide sample concentration. Then 50 μl of the peptide sample is transferred to a plastic vial and loaded onto a Waters Acuity UPLC-MS for crude analysis. The method used to analyze the crude peptides utilized a 0.5 ml/min flow rate on a Waters BEH 1.7 μm×100 mm column. The ACN:H₂O gradient ran for 10 min (10% to 80% ACN over 8 min) with an increase to 90% ACN from 10-11 min. Once target peptide retention time and mass are confirmed the peptide powders are then stored at −20° C. until needed for further reaction/conjugation or reverse phase purification.

### Example 2: Purification of Cyclized Alfa Tags

### Materials and Instruments

Preparative HPLC: Autopurification System with 2767 sample manaager, 2545 binary gradient manager, 515 pump for at-column dilution, 2489 UV detector, and SQD2 mass spectrometer (Waters Corporation)
UPLC-MS: Waters Acquity UPLC-MS with H-Class FTN Sample Manager, H-Class Quaternary Solvent Manger, Column Manager, Photodiode Array UV Detector, and QDa Mass Detector
Purification Column: Phenomenex Luna C18(2) LC Column, 100 Å pore size, 10 μm particle size, 30 mm×250 mm (Part #: 00G-4253-U0-AX)
Salt Exchange Preparative Column: Waters XSelect Peptide CSH C₁₈ OBD Preparative Column, 130 Å pre size, 5 μm particle size, 19×100 mm (Part #: 186007020)
Analytical Column: Waters Acquity UPLC Peptide BEH C18 Column, 130 Å pore size, 1.7 μm particle size, 2.1 mm×100 mm (Part #186003555)
Water: Honeywell HPLC Grade Water (Cat. AH365-4); or in-house Sartorius Arium Pro Water Purification System
Acetonitrile: Sigma-Aldrich HPLC Grade Acetonitrile (Cat. 34851-4L); or The Lab Depot LABSOLV LC/MS Acetonitrile (cat. LSL1140)
Trifluoroacetic Acid (TFA): Sigma-Aldrich HPLC Grade TFA (Cat. 302031-1L)
Dimethyl Sulfoxide (DMSO): Sigma-Aldrich Dimethyl Sulfoxide (Cat. D1435-500ML)
Ammonium Acetate: Sigma Life Science Ammonium Acetate (Cat. A1542-250G)
Acetic Acid: Mallinckrodt Chemicals Glacial Acetic Acid (V193-46)

Crude Sample Preparation for Purification: Lyophilized crude peptides were dissolved in DMSO or 50/50 water/acetonitrile to about 50 mg/mL They were vortexed and sonicated until fully dissolved (no visual presence of solid material). Dissolved peptides were pipetted into a sample plate for HPLC injection.

Purification: Peptides were loaded onto the preparative HPLC column with an automatic injector through a 10 mL sample loop. Sample loading was done in 95% mobile phase A, 5% mobile phase B. Peptide elution was performed in a 36-minute gradient with a mobile phase B increase of 15% (0.4%/min). The gradient mobile phase B composition was determined by a crude peptide UPLC retention time correlation factor. The SQD2 mass spectrometer was used to trigger fraction collection automatically based on the M+1H, M+2H, M+3H values for the target peptide. UV₂₁₄ absorption was monitored but not used for fraction triggering. Purification HPLC Conditions are defined in Table 3.

TABLE 3
Preparative HPLC Conditions

| Parameter | Value |
|---|---|
| Column | Phenomenex Luna C18(2) LC Column, |
| | 100 Å pore size, 10 μm particle size, 30 |
| | mm × 250 mm |
| Column Temperature | Room Temperature |
| Sample Temperature | Room Temperature |
| Sample Injection Volume | 1-10 mL |
| Mobile Phase A | 0.05% TFA in water |
| Mobile Phase B | 0.05% % TFA in acetonitrile |
| Flow Rate | 30 mL/min |
| Gradient | Starting % mobile phase B → Starting % |
| | mobile phase B + 15% in 36 minutes |
| UV Wavelength | 214 nm |
| UV Sampling Rate | 1 Hz |
| MS Mode | ES+ |
| MS Range | 200-3000 Da |
| MS Cone Voltage | 60 V |
| MS Capillary Voltage | 3.5 kV |
| MS Probe Temperature | 600° C. |
| MS Sampling Rate | 2 Hz |

In-Process Purification QC: Aliquots of selected peptide fractions were pipetted into a 96 well plate and injected on the UPLC-MS. The UPLC-MS conditions used for in-process fraction QC are listed in Table 4 For some peptides, in-process fraction QC was omitted by visually monitoring the preparative UV₂₁₄ absorption and MS to estimate which fractions were high purity.

TABLE 4
UPLC Conditions

| Parameter | Value |
|---|---|
| Column | Waters Acquity UPLC Peptide BEH C₁₈ |
| | Column, 130 Å pore size, 1.7 μm particle |
| | size, 2.1 mm × 100 mm |
| Column Temperature | 40° C. |
| Sample Temperature | 10° C. |
| Sample Injection Volume | 1-10 μL |
| Mobile Phase A | 0.100% TFA in water |
| Mobile Phase B | 0.085% TFA in acetonitrile |
| Flow Rate | 0.5 mL/min |
| Gradient | 10% → 90% B in 8 minutes, followed by 1 |
| | minute wash at 90% B, then 2 minute re- |
| | equilibration at 10% B |
| UV Wavelength | 214 nm |
| UV Sampling Rate | 10 Hz |
| MS Mode | ES+ |
| MS Range | 200-1250 Da |
| MS Cone Voltage | 15 V |
| MS Capillary Voltage | 0.8 kV |
| MS Probe Temperature | 600° C. |
| MS Sampling Rate | 2 Hz |

Fraction Pooling: Fractions with purity ≥90% were combined into tared 50 mL polypropylene tubes and lyophilized. If the pure fraction volume exceeded the tube capacity, multiple polypropylene tubes were filled and lyophilized. Then each peptide was resuspended in 50/50 water/acetonitrile+0.05% TFA and combined into one tared polypropylene tube and lyophilized.

Salt Exchange: For in vivo applications, the peptide counterion was exchanged from trifluoroacetate to acetate. In this instance, the peptide was dissolved in ammonium bicarbonate (10 g/L), vortexed and sonicated until fully dissolved (no visual presence of solid material) and pipetted into a sample plate for HPLC injection. The peptide was loaded onto the salt exchange HPLC column with an automatic injector through a 10 mL sample loop with 100% ammonium bicarbonate (10 g/L). Ammonium bicarbonate was flowed over the column for 20 minutes at 15 mL/minute. Then 1% v/v acetic acid in water was flowed over the column for 20 minutes. Finally, the peptide was eluted with a sharp gradient from 0 to 60% acetonitrile in 15 minutes. Peptide collection was triggered by the mass spectrometer, which was being used to monitor the M+1H, M+2H, M+3H values for the target peptide. Fractions were combined into tared polypropylene tubes and lyophilized.

Lyophilization: Peptide solutions were frozen on dry ice for at least 2 hours. They were then lyophilized with pressure <150 mTorr. After at least 48 hours, peptides were removed from the lyophilizer and inspected to ensure they were white powders with no liquid visually present.

Peptide Weighing: Following lyophilization, the polypropylene tubes containing peptide powders were weighed on the analytical balance. The tube tare weight was subtracted, and the weights were recorded with tenth of a milligram precision.

Final QC Peptide final QC was run on the H-Class UPLC-MS. Each peptide was dissolved to about 1 mg/mL in 0.05% TEA in water and injected on the UPLC. The UPLC/MS conditions were the same as those used for in-process purification QC and are defined in Table 4. Peptide purity was determined by relative UV area at 214 nm and identity was determine by mass spectrometry Acceptance criteria were that each peptide had UV₂₁₄ purity ≥90% and molecular weight within 1 AMU of theoretical MW. Results for each peptide are shown in Table 5

TABLE 5
Peptide QC Characterization Data

| | Theoretical | Experimental | UV214 | Salt |
|---|---|---|---|---|
| Peptide Sequence | MW | MW | Purity (%) | Form |
| Ac-Pro-Ser-Arg-Leu-Glu-cyclo(Glu-Glu- | 1779.0 | 1778.8 | 100 | Acetate |
| Leu-Arg-Lys)-Arg-Leu-Thr-Glu-NH₂ | | | | |
| Ac-Pro-Ser-Arg-Leu-Glu-cyclo(Lys-Glu- | 1779.0 | 1778.8 | 94 | TFA |
| Leu-Arg-Glu)-Arg-Leu-Thr-Glu-NH₂ | | | | |
| Ac-Pro-Ser-Arg-Leu-cyclo(Glu-Glu-Glu- | 1779.0 | 1778.8 | 99 | TFA |
| Leu-Lys)-Arg-Arg-Leu-Thr-Glu-NH₂ | | | | |
| Ac-Pro-Ser-Arg-Leu-Glu-cyclo(Glu-Glu- | 1779.0 | 1779.4 | 100 | TFA |
| Leu-Arg-dLys)-Arg-Leu-Thr-Glu-NH₂ | | | | |
| Ac-Pro-Ser-Arg-Leu-Glu-cyclo(Cys- | 1744.0 | 1743.6 | 100 | TFA |
| Glu-Leu-dCys)-Arg-Arg-Leu-Thr-Glu- | | | | |
| NH₂ | | | | |
| Ac-Ser-Arg-Leu-Glu-cyclo(Glu-Glu-Leu- | 1681.9 | 1681.6 | 99 | TFA |
| Lys)-Arg-Arg-Leu-Thr-Glu)-NH₂ | | | | |
| Ac-Ser-Arg-Leu-Glu-Glu-Glu-Leu-Arg- | 1681.9 | 1681.6 | 98 | TFA |
| cyclo(Lys-Arg-Leu-Thr-Glu)-NH₂ | | | | |
| Ac-Ser-Arg-Leu-Glu-cyclo(Glu-Glu-Leu- | 1681.9 | 1681.6 | 97 | TFA |
| Arg-Lys)-Arg-Leu-Thr-Glu-NH₂ | | | | |
| Ac-Ser-Arg-Leu-Glu-cyclo(Asp-Glu- | 1667.9 | 1667.4 | 95 | TFA |
| Leu-Arg-Lys)-Arg-Leu-Thr-Glu-NH₂ | | | | |

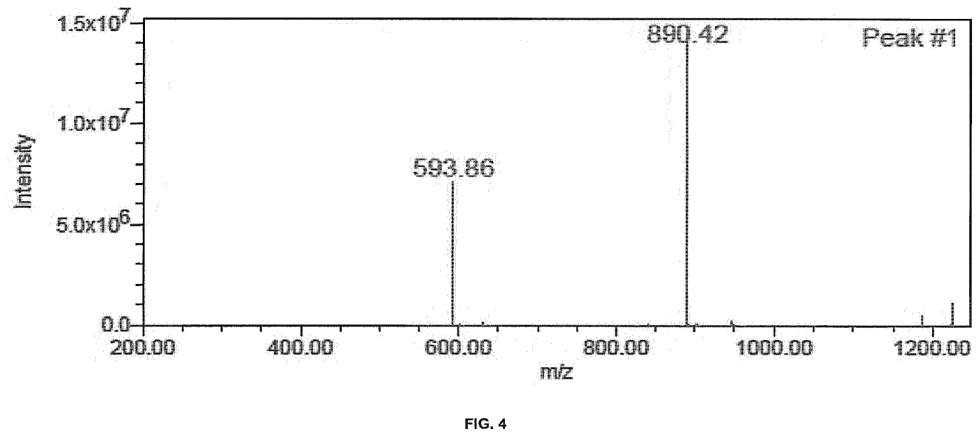

UPLC-UV₂₁₄ and UV₂₁₄ purity (100%) for the peptide Ac-Pro-Ser-Arg-Leu-Glu-cyclo(Glu-Glu-Leu-Arg-Lys)-Arg-Leu-Thr-Glu-NH₂ (MW: 1778.8) is shown in FIGS. 3 and 4, respectively.

### Example 3: Cell Binding Analysis of Linear and Cyclic ALFA-Peptides

The following example shows the Fluorescent Activated Cell Source (FACS) data of linear and cyclic ALFA-peptide (conjugated to AlexaFluor680) on cells.

Cells were seeded in a 96-well microtiter plate (round button) to a final density of 2×10⁵ and incubated in the presence of 100 nM NbALFA×anti-Target purified Adaptor-Protein (binds with one moiety to ALFA peptide and with the other moiety to cell presented target) or, as a negative control in the presence of 100 nM NbALFA×non-binder (binds with one moiety to the ALFA peptide and with the other moiety to a target which is not available in this experiment) or PBS for 30 min at 4° C. Cells were then washed and incubated with different concentrations (4 nM, 16 nM, 64 nM, 256 nM, and 1024 nM) of the linear [Ac(C-Alexa680)(PEG6)PSRLEEELRRRLTE-NH₂] or cyclic [Ac(C-Alexa680)(PEG6)PSRLE(E)ELR(K)RLTE-NH₂] ALFA peptide for another 30 min at 4° C. After rewashing, the cells were measured in FACS Canto II (BD Biosciences). AlexaFluor680 signal was used to detect the binding of ALFA peptides. Therefore, excitation Laser Line 633 nm was used to detect the AlexaFluor 680 signal.

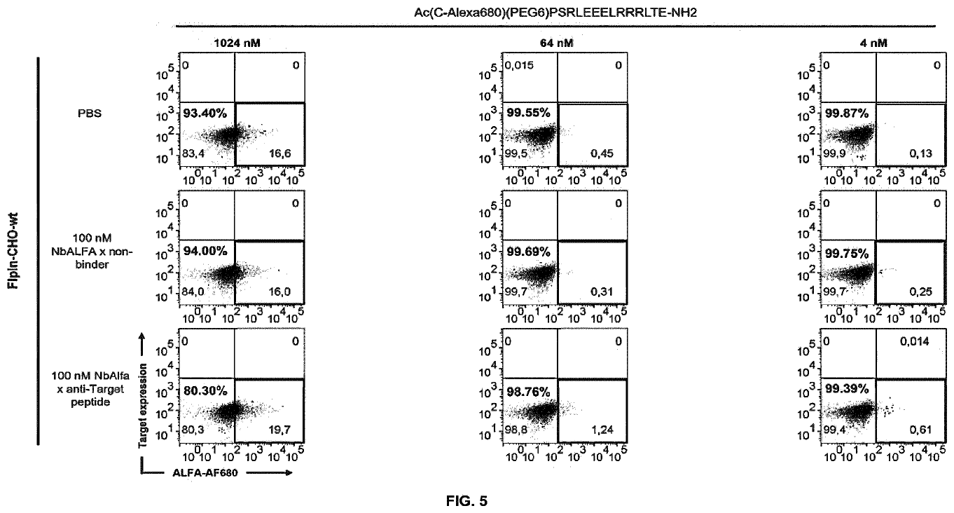

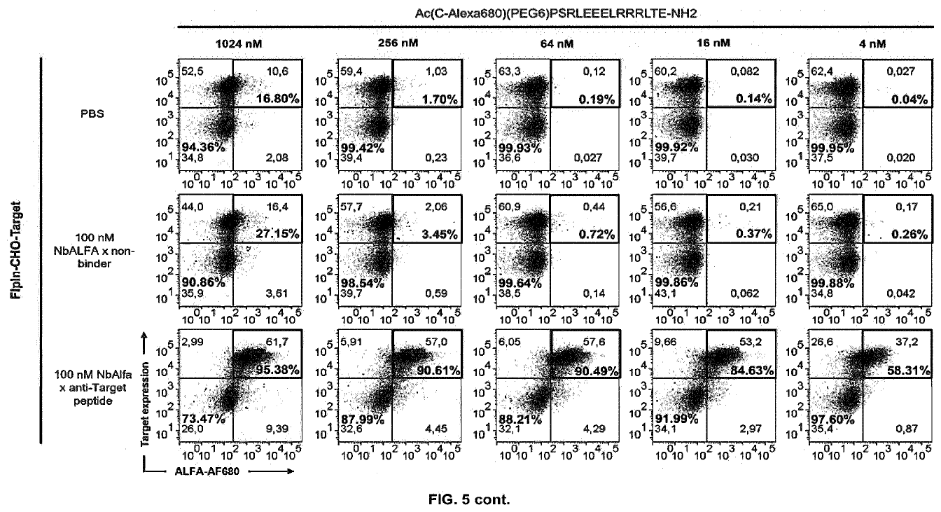

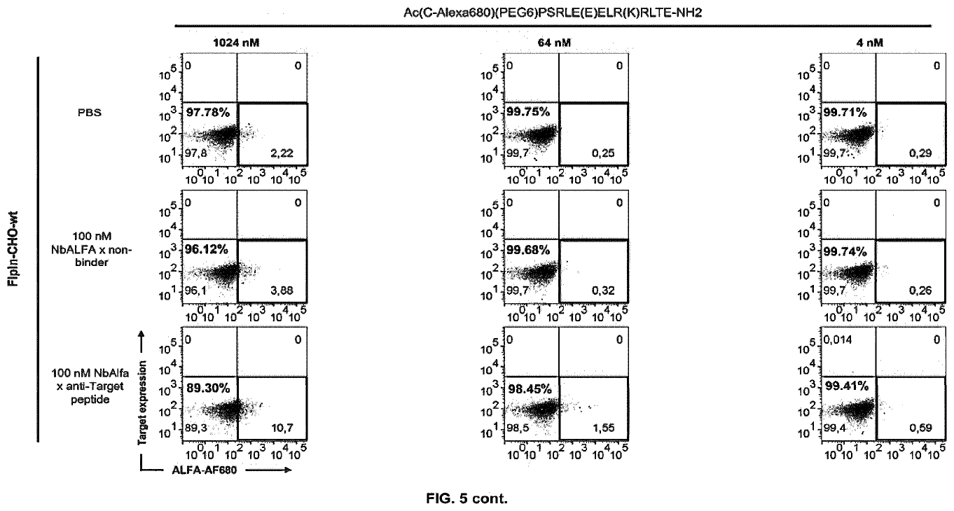

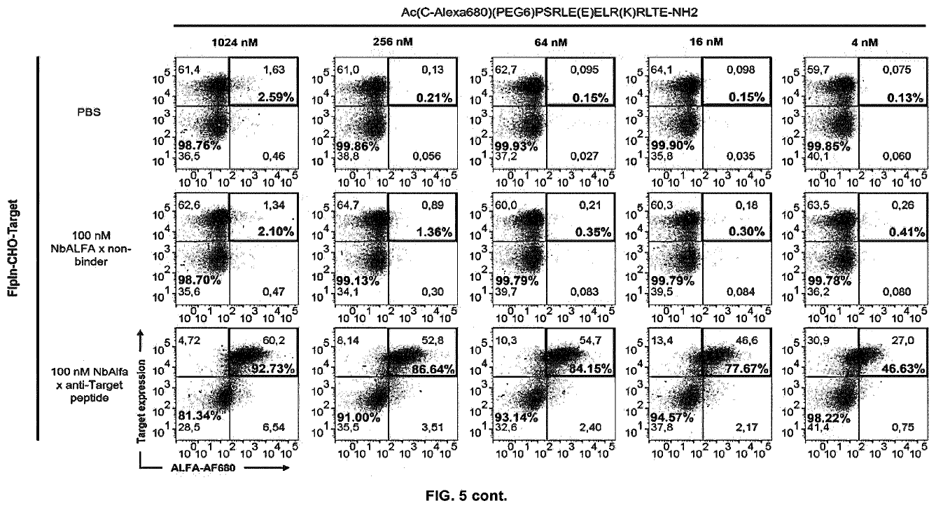

Referring now to FIG. 5, FACS data shows that both linear and cyclic ALFA peptides bind only to target expressing cells previously incubated with the NbALFA×anti-Target Adaptor-Protein. No binding was observed on the controls (binding to target-negative cells and pre-incubation with PBS or NbALFA×non-binder). Therefore, binding of ALFA peptides to target cells does not occur non-specifically, but only in dependence of the NbALFA×anti-Target adaptor administered prior.

### Example 4: SPR Measurements of NbALFA Binding to (Cyclic-) ALFA

Affinity measurements were performed using a Biacore T200 (GE Healthcare, Biacore T200 control Software 3.2). For affinity measurement, the Biotin CAPture kit, series S (Cytiva Europe, Cat. no. 28920234) was utilized. A freshly docked Biotin CAPture chip was rehydrated overnight in the instrument (standby mode). The following day, the chip surface was conditioned three times with regeneration solution (6 M guanidine-HCl, 0.25 M NaOH) for 60 s at 10 μL/min on flow cell (FC) 1 and FC2. Subsequently, the Biotin CAPture reagent (Cytiva Europe, Cat. no. 29423383) was applied for 300 s at 2 μL/min on FC1 and FC2, followed by capturing of 25 nM biotinylated NbALFA (NanoTag). The immobilization of the Nanobody was done for 180 s at 10 μL/min on FC2 followed by quenching of unbound streptavidin by 0.1 mM biocytin for 60 s and 30 μL/min on both flow cells. Five injections of 10 nM, 5 nM, 2.5 nM, 1.25 nM, 0.625 nM and 0 nM (blank control) of cyclic ALFA variants were performed over both flow cells for 120 s with a flow rate of 40 μL/min, followed by dissociation for 1500 s. After each cycle, the surface was regenerated for 120 s with 10 μL/min using regeneration solution. All measurements were performed at 25° C. using HBS-EP+ buffer (Cytiva Europe, Cat. no BR100669). The control responses from FC1 were subtracted by the sample measurement in FC2 (FC2−FC1) followed by subtraction of the 0 nM blank injection. Resulting binding curves of this multi cycle kinetic experiment were fitted using the Biacore T200 Evaluation Software 3.2 and a 1:1 binding model.

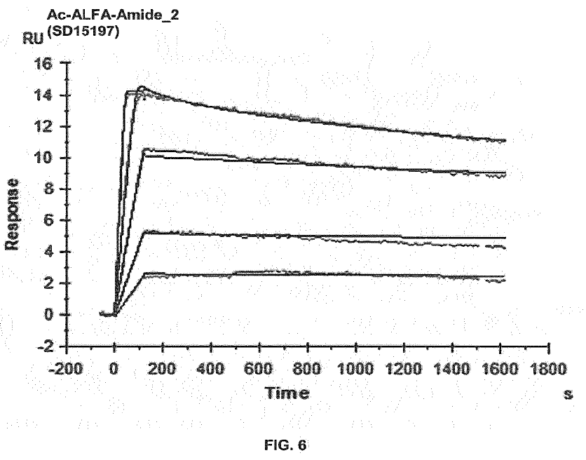

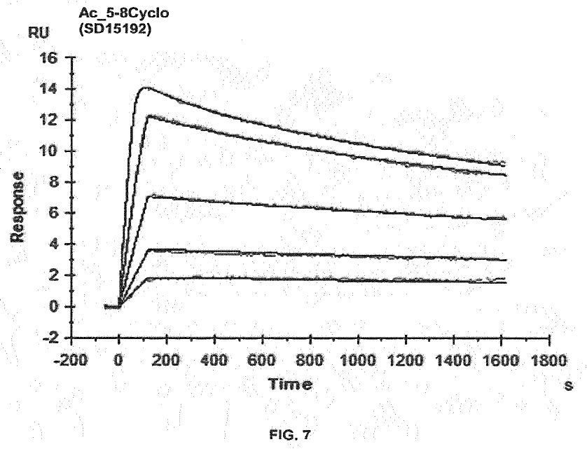

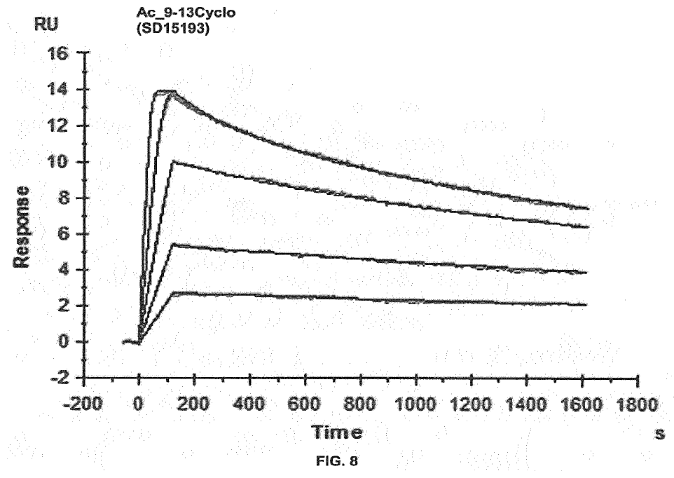

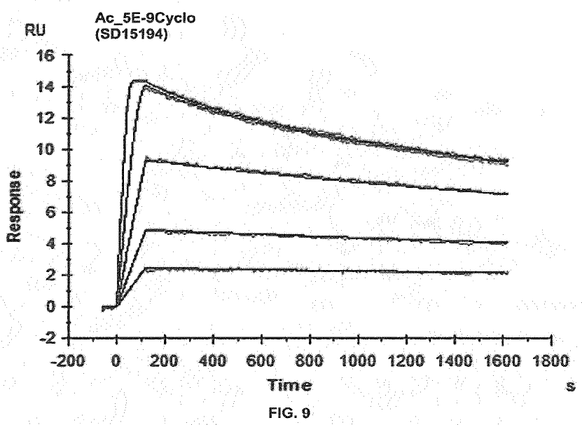

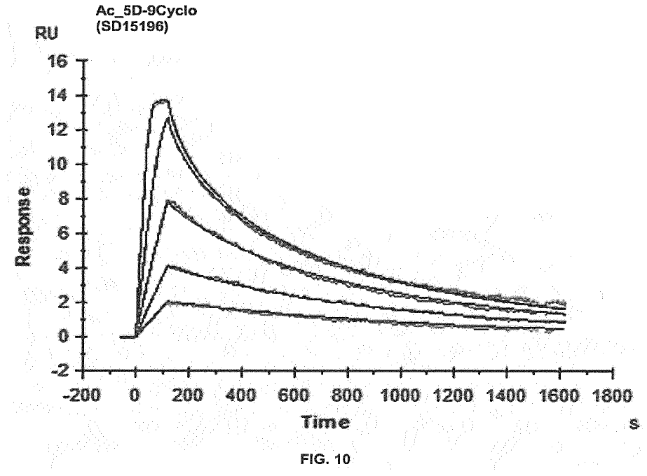

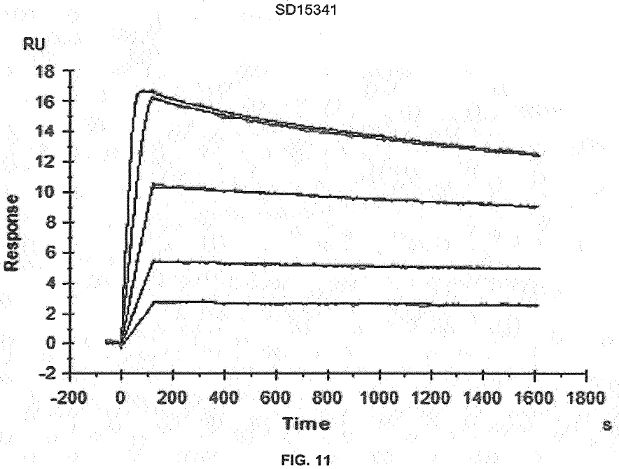

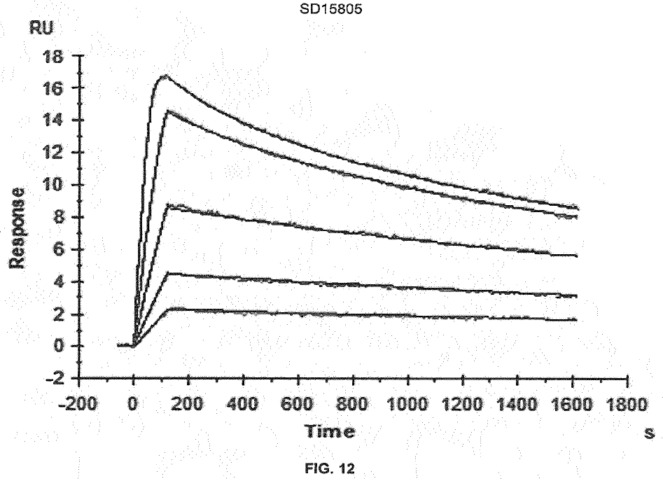

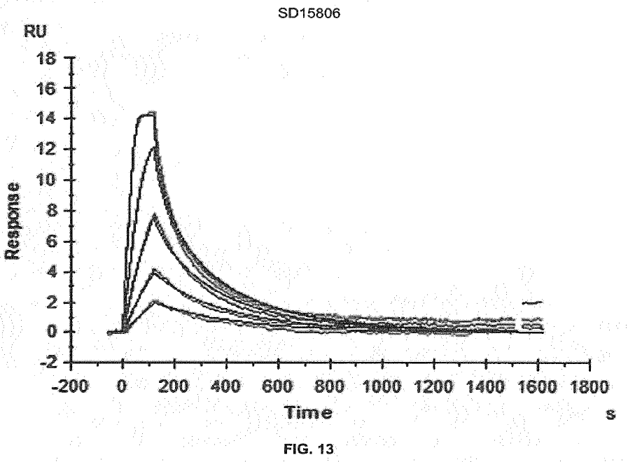

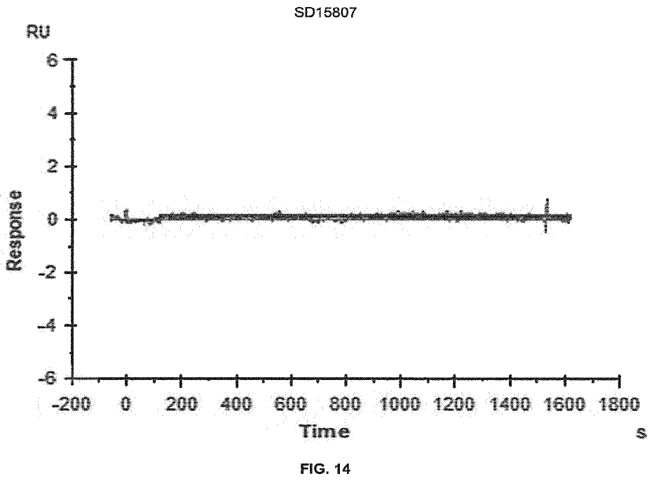

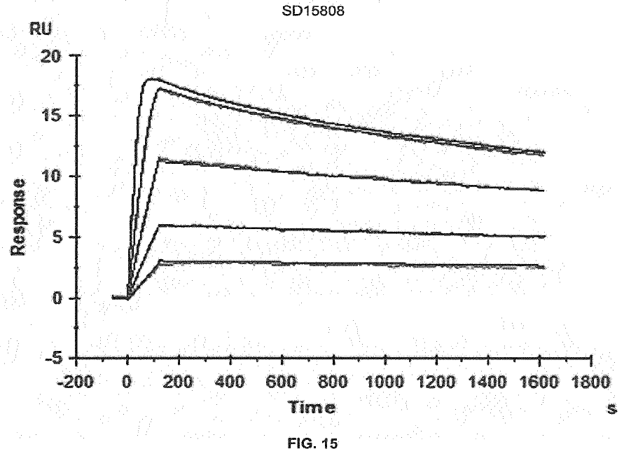

The results are shown in FIGS. 6-15 as well as Tables 6 and 7, where it can be seen that samples SD15194, SD15341, and SD15808 show very good binding with respect to NbALFA. Samples SD15194, SD15341, and SD15808 utilize the E5-K9 or the E6-K10 cyclization. Table 8 provides the structures for each sample tested.

TABLE 6

| Sample | ka (1/Ms) | kd (1/s) | KD (M) |
|---|---|---|---|
| Ac-ALFA-Amide_2 | 5.05E+07 | 5.93E−04 | 1.17E−11 |
| (SD15197) | | | |
| Ac_5-8Cyclo | 1.05E+07 | 5.77E−04 | 5.51E−11 |
| (SD15192) | | | |
| Ac_9-13Cyclo | 1.95E+07 | 1.09E−03 | 5.56E−11 |
| SD15193) | | | |
| Ac_5E-9Cyclo | 1.78E+07 | 6.70E−04 | 3.76E−11 |
| (SD15194) | | | |
| Ac_5D-9Cyclo | 1.81E+07 | 7.90E−03 | 4.36E−10 |
| (SD15196) | | | |

TABLE 7

| Sample | ka (1/Ms) | kd (1/s) | KD (M) |
|---|---|---|---|
| SD15341 | 1.86E+07 | 3.61E−04 | 1.94E−11 |
| SD15805 | 9.33E+06 | 9.64E−04 | 1.03E−10 |
| SD15806 | 3.44E+09 | 4.94E+00 | 1.43E−09 |
| SD15807 | n.d. | n.d. | n.d. |
| SD15808 | 1.35E+07 | 5.13E−04 | 3.80E−11 |

TABLE 8

| Sample | Peptide Sequence |
|---|---|
| SD15197 | Ac-Ser-Arg-Leu-Glu-Glu-Glu-Leu-Arg-Arg-Arg-Leu-Thr-Glu-NH₂ |
| SD15192 | Ac-Ser-Arg-Leu-Glu-(cyclo5)Glu-Glu-Leu-(cyclo8)Lys-Arg-Arg- |
| | Leu-Thr-Glu-NH₂ |
| SD15193 | Ac-Ser-Arg-Leu-Glu-Glu-Glu-Leu-Arg-(cyclo9)Lys-Arg-Leu-Thr- |
| | (cyclo13)Glu-NH₂ |
| SD15194 | Ac-Ser-Arg-Leu-Glu-(cyclo5)Glu-Glu-Leu-Arg-(cyclo9)Lys-Arg- |
| | Leu-Thr-Glu-NH₂ |
| SD15196 | Ac-Ser-Arg-Leu-Glu-(cyclo5)Asp-Glu-Leu-Arg-(cyclo9)Lys-Arg- |
| | Leu-Thr-Glu-NH₂ |
| SD15341 | Ac-Pro-Ser-Arg-Leu-Glu-(cyclo6)Glu-Glu-Leu-Arg-(cyclo10)Lys-Arg- |
| | Leu-Thr-Glu-NH₂ |
| SD15805 | Ac-Pro-Ser-Arg-Leu-Glu-(cyclo6)Lys-Glu-Leu-Arg-(cyclo10)Glu- |
| | Arg-Leu-Thr-Glu-NH₂ |
| SD15806 | Ac-Pro-Ser-Arg-Leu-(cyclo5)Glu-Glu-Glu-Leu-(cyclo9)Lys-Arg- |
| | Arg-Leu-Thr-Glu-NH₂ |
| SD15807 | Ac-Pro-Ser-Arg-Leu-Glu-(cyclo6)Cys-Glu-Leu-(cyclo9)dCys-Arg- |
| | Arg-Leu-Thr-Glu-NH₂ |
| SD15808 | Ac-Pro-Ser-Arg-Leu-Glu-(cyclo6)Glu-Glu-Leu-Arg-(cyclo10)dLys- |
| | Arg-Leu-Thr-Glu-NH₂ |

### Example 5: Pharmakinetic (PK) Study (Mouse)

### Animals and Animal Housing

C57BL/6NRj mice were obtained from Janvier Labs, France. In this study, approximately 8-week-old female mice were used. Animal housing conditions are listed in Table 9.

TABLE 9
Animal Housing Conditions

| | |
|---|---|
| Accommodation | Macrolon type III H cages; |
| | in Scantainer incubator 50-SCNT-Q-110 with a |
| | minimum of 25 air changes/hour |
| Housing density | groups of up to 9 mice/cage |
| Temperature | 20 to 26° C. |
| Relative humidity | 40 to 60% |
| Light/dark cycle | 12 hours/12 hours light/dark |
| Bedding | aspen wood granules, 2-3 mm |
| Environmental | wooden aspen chew blocks, certified nesting |
| Enrichment | material, polycarbonate or paper pulp huts |
| Diet | ad libitum access to “V1536-003 ssniff R/M-H, |
| | extrudiert” (Ssniff, Ratte Maus Haltung) |
| Water | tap water, ad libitum access to water bottles |
| Food/water restriction | no |

### Animal Protocol

The study was performed in strict accordance with national and European guidelines for the care and use of laboratory animals (DIRECTIVE 2010/63/EU OF THE EUROPEAN PARLIAMENT AND OF THE COUNCIL of 22 Sep. 2010 on the protection of animals used for scientific purposes).

All procedures in this study were carried out in compliance with the German Animal Welfare Act and German regulations (TierSchG/TierSchVersV), and were approved by the local authority (Regierung von Oberbayern, München).

### PK Study

Compounds used for intravenous bolus injection were formulated in an aqueous glucose solution (SD14316 in 4% glucose, SD15341 in 5% glucose). Prior to administration into the lateral tail vein, the body weight of the animals was determined to calculate the individual volume of the injecting compound (5 mL/kg body weight). The injection, as well as the blood sampling, were carried out under short anesthesia (0.7 L/min O₂, 1.7 L/min N₂O, 2% Isoflurane). Blood was sampled from the retro-orbital plexus using microhematocrit capillary (Ø 0.8 mm) and collected into K₃EDTA tubes, at the time points indicated in Table 10.

TABLE 10
Time points for blood sampling after injection

| Compound | Time points for blood sampling |
|---|---|
| SD14316 (Linear Alpha) | 5 min, 10 min, 20 min, 0.5 h, 1 h, 1.5 h, 2 h, |
| | 4 h |
| SD15341 (Cyclic Alpha) | 1 min, 3 min, 5 min, 10 min, 20 min, 0.5 h, |
| | 0.75 h, 1 h |

The plasma was separated by centrifugation (10,000×g; 10 min; 4° C.), aliquoted into microtubes and stored at −80° C.

### LC-MS/MS Quantification

20 μl of each plasma sample was transferred into PCR tubes. 80 μl IS (internal standard)-solution in acetonitrile+1% FA was added for protein precipitation.

The sample tubes were sealed with pierceable cap mats and vortexed. After homogenization for 5 min in an ultrasonic bath the samples were centrifuged at 3000 rpm at ambient temperature for 15 min. 1 μl supernatant was injected into the instrument for LC-MS/MS analysis. For LC-MS/MS quantification a ExionLC™ UHPLC coupled to a QTRAP® 6500+ mass spectrometer (Sciex) was used in ESI positive mode. Parameters for mass spectrometry were listed in the tables below, Chromatographic separation was achieved on a ACQUITY UPLC BEH C18 column (Waters, 1.7 μm, 2.1×50 mm). Mobile phase composition, gradient information, mass transitions and spectrometer settings are tabulated below.

Compound concentrations in plasma samples were calculated using a 10-point calibration curve in plasma over a concentration range of 1 to 1000 ng/ml. QC samples of 1, 10, 100, 1000 ng/ml were analyzed before and after each dose group. Samples exceeding the upper limit of quantification (1000 ng/ml) were re-analyzed after 10-fold dilution in blank plasma. All calculations were performed in Sciex OS Version 1.7.0.36606 and MS-EXCEL®.

Pharmacokinetic parameters were calculated for both compounds using the software Kinetica (version 4.4.1, Thermo Scientific) applying a non-compartmental model (NCA).

TABLE 11
Parameters for Mass Spectrometry

| Parameter | Set |
|---|---|
| CUR | 30.00 |
| IS | 3500.00 |
| TEM | 550.00 |
| GS1 | 50.00 |
| GS2 | 50.00 |
| CAD | Medium |
| EP | 10.00 |
| Resolution Q1: | Unit |
| Resolution Q3: | Unit |

TABLE 12
mrm Reaction for SD14316

| Transition | Dwell(msec) | DP | CE | CXP | Analyte |
|---|---|---|---|---|---|
| 1: 609.145 > 603.300 | 100.00 | 25.00 | 35.00 | 24.00 | SD14316 |
| | | | | | (Quantifier) |
| 2: 609.145 > 594.900 | 100.00 | 25.00 | 37.00 | 24.00 | SD14316 |
| 3: 573.807 > 571.100 | 50.00 | 25.00 | 33.00 | 24.00 | IS (SD15197) |
| | | | | | (used for |
| | | | | | quantification) |
| 4: 573.807 > 562.700 | 50.00 | 25.00 | 33.00 | 20.00 | IS (SD15197) |

TABLE 13
mrm Reaction for SD15341

| Transition | Dwell(msec) | DP | CE | CXP | Analyte |
|---|---|---|---|---|---|
| 1: 593.846 > 227.200 | 50.00 | 50.00 | 50.00 | 18.00 | SD15341 |
| | | | | | (Quantifier) |
| 2: 593.846 > 812.200 | 125.00 | 50.00 | 30.00 | 38.00 | SD15341 |
| 3: 576.807 > 571.100 | 125.00 | 50.00 | 30.00 | 24.00 | IS (SD15197) |
| | | | | | (used for |
| | | | | | quantification) |
| 4: 576.807 > 562.700 | 100.00 | 50.00 | 40.00 | 22.00 | IS (SD15197) |

TABLE 14
Timetable for UHPLC Measurement of SD15341

| Time | Solvent A | Solvent B | Flow |
|---|---|---|---|
| 0.00 | 90.0 | 10.0 | 0.700 |
| 0.90 | 30.0 | 70.0 | 0.700 |
| 1.10 | 10.0 | 90.0 | 0.700 |
| 1.60 | 10.0 | 90.0 | 0.700 |
| 1.70 | 90.0 | 10.0 | 0.700 |

TABLE 15
Timetable for UHPLC Measurement of SD15341

| Time | Solvent A | Solvent B | Flow |
|---|---|---|---|
| 0.00 | 90.0 | 20.0 | 0.700 |
| 0.90 | 30.0 | 70.0 | 0.700 |
| 1.10 | 10.0 | 90.0 | 0.700 |
| 1.60 | 10.0 | 90.0 | 0.700 |
| 1.70 | 90.0 | 20.0 | 0.700 |

TABLE 16
Data of SD14316 and SD115341

| | Compound | | | Exact mass | |
|---|---|---|---|---|---|
| | ID | Batch ID | Container ID | (g/mol) | Formula |
| Analyte | SD14316 | B1 00068775 | K20050994 | 1824.00 | C76H133N27O25 |
| Analyte | SD15341 | B1 00070316 | K20050994 | 1777.98 | C76H131N25O24 |
| Internal | SD15197 | B1 00069914 | K20048553 | 1726.94 | C71H126N26O24 |
| Standard (IS) | | | | | |

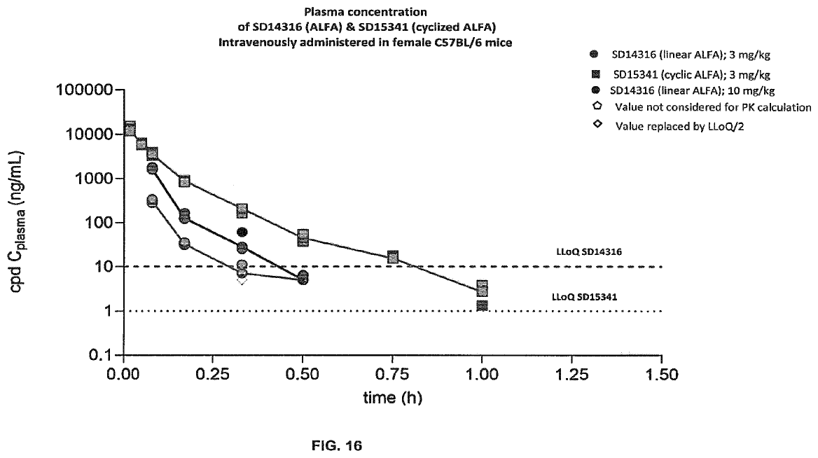

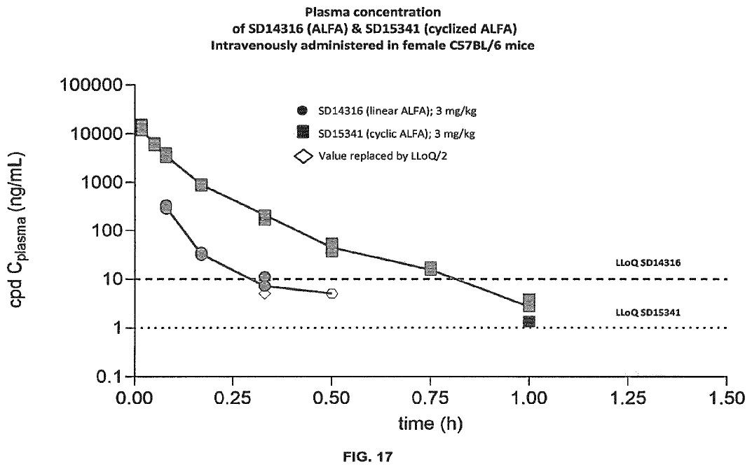

The results are shown in FIGS. 16 and 17 as well as Tables 17 and 18, which show that the cyclic alpha compound has a much higher area under the curve (AUC) and a drastically reduced clearance compared to the linear alpha compound.

TABLE 17
Non-compartment analysis after single i.v. treatment

| | SD14316 | SD15341 | SD14316 |
|---|---|---|---|
| Parameters | (3 mg/kg) | (3 mg/kg) | (10 mg/kg) |
| AUC₀₋ₜ (ng · h/mL) | 51 | 947 | 230 |
| AUC_{0-inf} (ng · h/mL) | 52⁽¹⁾ | 948 | 230 |
| AUCₑₓₜᵣₐ (%) | 1.5⁽¹⁾ | 0.05 | 0.2 |
| C₀ (ng/mL) | 656⁽¹⁾ | 15401 | 2395⁽¹⁾ |
| Cₘₐₓ (ng/mL) | 306 | 12714 | 1660 |
| tₘₐₓ (min) | 5.0 | 1.0 | 5.0 |
| V_{c} (mL/kg) | 4574⁽¹⁾ | 195 | 4175⁽¹⁾ |
| V_{z} (mL/kg) | 6303⁽¹⁾ | 509 | 4482 |
| Vₛₛ (mL/kg) | 3890 | 237 | 2946 |
| CL (mL/(h · kg)) | 57706 | 3165 | 43418 |
| λᵢₙᵢₜᵢₐₗ (min⁻¹) | 0.45⁽²⁾ | 0.29 | 0.52⁽²⁾ |
| R²ᵢₙᵢₜᵢₐₗ | 1.000 | 0.99 | 1.000 |
| λₜₑᵣₘᵢₙₐₗ (min⁻¹) | 0.15⁽²⁾ | 0.10 | 0.16 |
| R²ₜₑᵣₘᵢₙₐₗ | 1.000 | 0.98 | 1.00 |
| MRT₀₋ₜ (min) | 3.7 | 4.47 | 4.0 |
| MRT_{0-inf} (min) | 4.0 | 4.50 | 4.1 |
| t_{1/2initial} (min) | 1.6⁽¹⁾ | 2.41 | 1.3⁽¹⁾ |
| t_{1/2terminal} (min) | 4.5⁽¹⁾ | 6.69 | 4.3 |
| t_{1/2effective} (min) | 2.6⁽¹⁾ | 3.10 | 2.8 |
| Time interval | 5-10 | 1-10 | 5-10 |
| to determine | | | |
| t_{1/2initial} (min) | | | |
| Time interval | 10-20 | 20-60 | 10-30 |
| to determine | | | |
| t_{1/2terminal} (min) | | | |

⁽¹⁾approximate value, as terminal phase could only be defined by two data points (R² for λ_{z} = 1)
⁽²⁾was calculated by extrapolation of only two data points (R² = 1)

TABLE 18
Normalized to a dose of 1 mg/kg

| | SD14316 | SD15341 | SD14316 |
|---|---|---|---|
| Parameter | (3 mg/kg) | (3 mg/kg) | (10 mg/kg) |
| AUC₀₋ₜ | 17 | 316 | 23 |
| (ng · h/mL) | | | |
| AUC_{0-inf} | 17 | 316 | 23 |
| (ng · h/mL) | | | |
| C₀ (ng/mL) | 219⁽¹⁾ | 5134 | 240⁽¹⁾ |
| Cₘₐₓ (ng/mL) | 102 | 4238 | 166 |
| F (%) | — | — | — |

⁽¹⁾approximate value, as terminal phase could only be defined by two data points (R² for λ_{z} = 1)
⁽²⁾was calculated by extrapolation of only two data points (R² = 1)
Volume of distribution (Vd) in mice:
~50 mL/kg = plasma volume
~85 mL/kg = blood volume
~725 mL/kg = total body water (TBW)
>TBW = high tissue distribution

### Example 6: Plasma Stability—Human and Mouse

Plasma stability measurements were performed by spiking 5 μl of a 1 mM compound stock solution in DMSO into 495 μl of human or mouse Li-heparin plasma (Innovative Research, Cat. No, IPLALIH5OML (human) and IMS-BC-N (Balb C mouse). The spiked plasma was distributed in 50 μl aliquots into capped PCR tubes for incubation at 37° C. At each time point (0, 2, 6, 24 hours*) 3*aliquots were withdrawn from the incubator. Enzymatic reactions were quenched directly after withdrawal by adding 100 μl of acetonitrile/formic acid=99/1 (v/v). Quenched samples were stored at ambient temperature until the end of the incubation period (24 hrs*). After centrifugation, the supernatant was transferred into fresh vials and analyzed by LC-MS/MS in ESI positive mode (Waters ACQUITY UPLC I-Class System (SM-FTN) with Xevo TQD triple quadrupole mass spectrometer with control software Waters MassLynx). Chromatographic separation was achieved on a Waters Cortex T3 analytical column (1.6 μm particle size, 2.1×30 mm). Mobile phase composition, gradient information, mass transitions and spectrometer settings are tabulated below Compound concentration in assay samples after incubation were calculated using a 3-point calibration curve in acetonitrile/water=2/1 (v/v) over a concentration range of 0.1 to 10 μM. Values below the limit of quantification (0.1 μM) and outliers of triplicate results were excluded from further calculations. An exponential regression curve was fitted onto the concentration results. Half-life times were calculated from the slope of the regression curve (t_{1/2}=ln(2)/slope).

For some samples, as indicated below, the incubation time was 6 hours. The 24 hour data was measured in triplicates whereas the 6 hour data was measured in duplicates.

Tables 19 to 21 provide the LC-MS/MS instrument settings for each sample tested.

TABLE 19

| | | | | Capillary | Cone | Collision |
|---|---|---|---|---|---|---|
| | | | Mass transition | voltage | voltage | energy |
| Internal ID | Description | Mobile phase | (m/z) | (kV) | (V) | (eV) |
| SD 00014316 | Ac-Pro-ALFA-NH2 | A: 0.1% formic acid | 609.341 > 112.168 | 1 | 34 | 38 |
| | | B: 0.1% formic acid in acetonitrile | | | | |
| SD 00015192 | Ac-Ser-Arg-Leu-Glu-(cyclo5)Glu-Glu-Leu- | A: 0.1% formic acid | 841.566 > 70.076 | 1.2 | 44 | 70 |
| | (cyclo8)Lys-Arg-Arg-Leu-Thr-Glu-NH2 | B: 0.1% formic acid in acetonitrile | | | | |
| SD 00015193 | Ac-Ser-Arg-Leu-Glu-Glu-Glu-Leu-Arg- | A: 0.1% formic acid | 841.566 > 70.075 | 1.2 | 48 | 74 |
| | (cyclo9)Lys-Arg-Leu-Thr-(cyclo13)Glu-NH2 | B: 0.1% formic acid in acetonitrile | | | | |
| SD 00015194 | Ac-Ser-Arg-Leu-Glu-(cyclo5)Glu-Glu-Leu-Arg- | A: 0.1% formic acid | 841.566 > 70.075 | 1.2 | 48 | 80 |
| | (cyclo9)Lys-Arg-Leu-Thr-Glu-NH2 | B: 0.1% formic acid in acetonitrile | | | | |
| SD 00015196 | Ac-Ser-Arg-Leu-Glu-(cyclo5)Asp-Glu-Leu-Arg- | A: 0.1% formic acid | 834.492 > 84.033 | 1.2 | 42 | 86 |
| | (cyclo9)Lys-Arg-Leu-Thr-Glu-NH2 | B: 0.1% formic acid in acetonitrile | | | | |
| SD 00015805 | Ac-Pro-Ser-Arg-Leu-Glu-(cyclo6)Lys-Glu-Leu- | A: NH4 formate 10 mM in water | 593.815 > 112.078 | 1 | 30 | 36 |
| | Arg-(cyclo10)Glu-Arg-Leu-Thr-Glu-NH2 | (pH 3.8) | | | | |
| | | B: Acetonitrile | | | | |
| SD 00015806 | Ac-Pro-Ser-Arg-Leu-(cyclo5)Glu-Glu-Glu-Leu- | A: NH4 formate 10 mM in water | 593.815 > 112.078 | 1 | 30 | 37 |
| | (cyclo9)Lys-Arg-Arg-Leu-Thr-Glu-NH2 | (pH 3.8) | | | | |
| | | B: Acetonitrile | | | | |
| SD 00015807 | Ac-Pro-Ser-Arg-Leu-Glu-(cyclo6)Cys-Glu-Leu- | A: NH4 formate 10 mM in water | 582.178 > 112.08 | 2 | 30 | 36 |
| | (cyclo9)dCys-Arg-Arg-Leu-Thr-Glu-NH2 | (pH 3.8) | | | | |
| | | B: Acetonitrile | | | | |
| SD 00015808 | Ac-Pro-Ser-Arg-Leu-Glu-(cyclo6)Glu-Glu-Leu- | A: NH4 formate 10 mM in water | 593.815 > 140.057 | 2 | 24 | 38 |
| | Arg-(cyclo10)dLys-Arg-Leu-Thr-Glu-NH2 | (pH 3.8) | | | | |
| | | B: Acetonitrile | | | | |

TABLE 20
Gradient composition

| Time | Solv. A | Solv. B | Flow |
|---|---|---|---|
| (min) | (%) | (%) | (ml/min) |
| initial | 95 | 5 | 0.7 |
| 0.3 | 95 | 5 | 0.7 |
| 0.7 | 70 | 30 | 0.7 |
| 0.9 | 0 | 100 | 0.7 |
| 1.4 | 0 | 100 | 0.7 |
| 1.5 | 95 | 5 | 0.7 |
| Injection | 0.5 | | |
| Volume | | | |
| (ul) | | | |

TABLE 21
General MS settings

| Parameter | Setting |
|---|---|
| Source Temperature (° C.) | 150 |
| Desolvation Temperature (° C.) | 600 |
| Cone Gas Flow (L/Hr) | 50 |
| Desolvation Gas Flow (L/Hr) | 600 |
| Collision Gas Flow (mL/Min) | 0.15 |

Table 22 provides the half-life times in human plasma.

TABLE 22

| | | Half-life | |
|---|---|---|---|
| Internal ID | description | time (h) | Flag |
| SD 00014316 | Ac-Pro-ALFA-NH2 | 10.76 | >6 h (assay |
| | | | exceeded) |
| SD 00015192 | Ac-Ser-Arg-Leu-Glu-(cyclo5)Glu-Glu-Leu- | 3.89 | |
| | (cyclo8)Lys-Arg-Arg-Leu-Thr-Glu-NH₂ | | |
| SD 00015193 | Ac-Ser-Arg-Leu-Glu-Glu-Glu-Leu-Arg- | 56.07 | >24 h (assay |
| | (cyclo9)Lys-Arg-Leu-Thr-(cyclo13)Glu-NH₂ | | exceeded) |
| SD 00015194 | Ac-Ser-Arg-Leu-Glu-(cyclo5)Glu-Glu-Leu-Arg- | 33.48 | >24 h (assay |
| | (cyclo9)Lys-Arg-Leu-Thr-Glu-NH₂ | | exceeded) |
| SD 00015196 | Ac-Ser-Arg-Leu-Glu-(cyclo5)Asp-Glu-Leu-Arg- | 14.52 | |
| | (cyclo9)Lys-Arg-Leu-Thr-Glu-NH₂ | | |
| SD 00015805 | Ac-Pro-Ser-Arg-Leu-Glu-(cyclo6)Lys-Glu-Leu- | 13.85 | >6 h (assay |
| | Arg-(cyclo10)Glu-Arg-Leu-Thr-Glu-NH₂ | | exceeded*) |
| SD 00015806 | Ac-Pro-Ser-Arg-Leu-(cyclo5)Glu-Glu-Glu-Leu- | 20.83 | >6 h (assay |
| | (cyclo9)Lys-Arg-Arg-Leu-Thr-Glu-NH₂ | | exceeded*) |
| SD 00015807 | Ac-Pro-Ser-Arg-Leu-Glu-(cyclo6)Cys-Glu-Leu- | 6.07 | >6 h (assay |
| | (cyclo9)dCys-Arg-Arg-Leu-Thr-Glu-NH₂ | | exceeded*) |
| SD 00015808 | Ac-Pro-Ser-Arg-Leu-Glu-(cyclo6)Glu-Glu-Leu- | 16.06 | >6 h (assay |
| | Arg-(cyclo10)dLys-Arg-Leu-Thr-Glu-NH₂ | | exceeded*) |

Table 23 provides the half-life times in mouse plasma.

TABLE 23

| | | Half-life | |
|---|---|---|---|
| Internal ID | description | time (h) | Flag |
| SD 00014316 | Ac-Pro-ALFA-NH₂ | 3.80 | |
| SD 00015192 | Ac-Ser-Arg-Leu-Glu-(cyclo5)Glu-Glu-Leu- | 11.78 | |
| | (cyclo8)Lys-Arg-Arg-Leu-Thr-Glu-NH₂ | | |
| SD 00015193 | Ac-Ser-Arg-Leu-Glu-Glu-Glu-Leu-Arg- | 18.54 | |
| | (cyclo9)Lys-Arg-Leu-Thr-(cyclo13)Glu-NH₂ | | |
| SD 00015194 | Ac-Ser-Arg-Leu-Glu-(cyclo5)Glu-Glu-Leu-Arg- | 35.69 | >24 h (assay |
| | (cyclo9)Lys-Arg-Leu-Thr-Glu-NH₂ | | exceeded) |
| SD 00015196 | Ac-Ser-Arg-Leu-Glu-(cyclo5)Asp-Glu-Leu-Arg- | 26.18 | >24 h (assay |
| | (cyclo9)Lys-Arg-Leu-Thr-Glu-NH₂ | | exceeded) |
| SD 00015805 | Ac-Pro-Ser-Arg-Leu-Glu-(cyclo6)Lys-Glu-Leu- | 7.29 | >6 h (assay |
| | Arg-(cyclo10)Glu-Arg-Leu-Thr-Glu-NH₂ | | exceeded*) |
| SD 00015806 | Ac-Pro-Ser-Arg-Leu-(cyclo5)Glu-Glu-Glu-Leu- | 14.92 | >6 h (assay |
| | (cyclo9)Lys-Arg-Arg-Leu-Thr-Glu-NH₂ | | exceeded*) |
| SD 00015807 | Ac-Pro-Ser-Arg-Leu-Glu-(cyclo6)Cys-Glu-Leu- | 4.32 | |
| | (cyclo9)dCys-Arg-Arg-Leu-Thr-Glu-NH₂ | | |
| SD 00015808 | Ac-Pro-Ser-Arg-Leu-Glu-(cyclo6)Glu-Glu-Leu- | 8.74 | >6 h (assay |
| | Arg-(cyclo10)dLys-Arg-Leu-Thr-Glu-NH₂ | | exceeded*) |

The stability results in human plasma for each sample tested are listed in Tables 24 to 32.

TABLE 24
SD 14316

| Timepoint | | | | | Conc. |
|---|---|---|---|---|---|
| (h) | Tube 1 | | Tube 2 | Mean | (% of T0) |
| 0 X | 2.38 | X | 2.36 | 2.37 | 100.0 |
| 1 X | 2.1 | X | 2.12 | 2.11 | 89.0 |
| 3 X | 1.86 | X | 1.88 | 1.87 | 78.9 |
| 6 X | 1.62 | X | 1.55 | 1.59 | 66.9 |

TABLE 25
SD 15192

| | | | | | | | | Conc. |
|---|---|---|---|---|---|---|---|---|
| Timepoint (h) | Tube 1 | | Tube 2 | | Tube 3 | | Mean | (% of T0) |
| 0 | X | 2.667 | X | 2.471 | X | 2.553 | 2.56 | 100.0 |
| 2 | X | 1.788 | X | 1.757 | X | 1.731 | 1.76 | 68.6 |
| 6 | X | 0.876 | X | 0.868 | X | 0.883 | 0.88 | 34.2 |
| 24 | | 0.031 | | 0.036 | | 0.046 | | |

TABLE 26
SD 15194

| | | | | | | | | Conc. |
|---|---|---|---|---|---|---|---|---|
| Timepoint (h) | Tube 1 | | Tube 2 | | Tube 3 | | Mean | (% of T0) |
| 0 | X | 2.215 | X | 2.271 | X | 2.302 | 2.26 | 100.0 |
| 2 | X | 2.086 | X | 2.052 | X | 2.117 | 2.09 | 92.1 |
| 6 | X | 1.884 | X | 2.057 | X | 2.041 | 1.99 | 88.1 |
| 24 | X | 1.322 | X | 1.377 | X | 1.368 | 1.36 | 59.9 |

TABLE 27
SD 15193

| | | | | | | | | Conc. |
|---|---|---|---|---|---|---|---|---|
| Timepoint (h) | Tube 1 | | Tube 2 | | Tube 3 | | Mean | (% of T0) |
| 0 | X | 2.799 | X | 2.845 | X | 2.795 | 2.81 | 100.0 |
| 2 | X | 2.769 | X | 2.647 | X | 2.717 | 2.71 | 96.4 |
| 6 | X | 2.774 | X | 2.678 | X | 2.527 | 2.66 | 94.5 |
| 24 | X | 2.387 | X | 1.983 | X | 1.888 | 2.09 | 74.2 |

TABLE 28
SD 15196

| | | | | | | | | Conc. |
|---|---|---|---|---|---|---|---|---|
| Timepoint (h) | Tube 1 | | Tube 2 | | Tube 3 | | Mean | (% of T0) |
| 0 | X | 2.314 | X | 2.424 | X | 2.518 | 2.42 | 100.0 |
| 2 | X | 2.188 | X | 2.234 | X | 2.212 | 2.21 | 91.4 |
| 6 | X | 1.843 | X | 1.803 | X | 1.545 | 1.73 | 71.5 |
| 24 | X | 0.844 | X | 0.761 | X | 0.694 | 0.77 | 31.7 |

TABLE 29
SD 15805

| Timepoint | | | | Conc. |
|---|---|---|---|---|
| (h) | Tube 1 | Tube 2 | Mean | (% of T0) |
| 0.00 x | 2.894 x | 2.777 | 2.836 | 100.0 |
| 1.00 x | 2.529 x | 2.485 | 2.507 | 88.4 |
| 3.00 x | 2.408 x | 2.304 | 2.356 | 83.1 |
| 6.00 x | 2.046 x | 2.045 | 2.046 | 72.1 |

TABLE 30
SD 15807

| Timepoint | | | | Conc. |
|---|---|---|---|---|
| (h) | Tube 1 | Tube 2 | Mean | (% of T0) |
| 0.00 x | 2.203 x | 2.230 | 2.217 | 100.0 |
| 1.00 x | 2.094 x | 2.092 | 2.093 | 94.4 |
| 3.00 x | 1.583 x | 1.740 | 1.662 | 75.0 |
| 6.00 x | 1.141 x | 1.130 | 1.136 | 51.2 |

TABLE 31
SD 15806

| Timepoint | | | | Conc. |
|---|---|---|---|---|
| (h) | Tube 1 | Tube 2 | Mean | (% of T0) |
| 0.00 x | 3.114 x | 2.965 | 3.040 | 100.0 |
| 1.00 x | 2.843 x | 2.874 | 2.859 | 94.0 |
| 3.00 x | 2.772 x | 2.486 | 2.629 | 86.5 |
| 6.00 x | 2.534 x | 2.417 | 2.476 | 81.4 |

TABLE 32
SD 15807

| Timepoint | | | | Conc. |
|---|---|---|---|---|
| (h) | Tube 1 | Tube 2 | Mean | (% of T0) |
| 0.00 x | 2.882 x | 3.034 | 2.958 | 100.0 |
| 1.00 x | 2.884 x | 2.860 | 2.872 | 97.1 |
| 3.00 x | 2.532 x | 2.660 | 2.596 | 87.8 |
| 6.00 x | 2.296 x | 2.298 | 2.297 | 77.7 |

The stability results in mouse plasma for each sample tested are listed in Tables 33 to 41.

TABLE 33
SD 14316

| | | | | | | | | Conc. |
|---|---|---|---|---|---|---|---|---|
| Timepoint (h) | Tube 1 | | Tube 2 | | Tube 3 | | Mean | (% of T0) |
| 0 | X | 2.459 | | 1.371 | X | 2.796 | 2.63 | 100.0 |
| 2 | | 1.262 | X | 1.829 | X | 1.99 | 1.91 | 72.7 |
| 6 | X | 0.937 | X | 0.877 | X | 0.847 | 0.89 | 33.8 |
| 24 | | 0.088 | | 0.082 | | 0.071 | | |

TABLE 34
SD 15192

| | | | | | | | | Conc. |
|---|---|---|---|---|---|---|---|---|
| Timepoint (h) | Tube 1 | | Tube 2 | | Tube 3 | | Mean | (% of T0) |
| 0 | X | 2.731 | X | 2.705 | X | 2.658 | 2.70 | 100.0 |
| 2 | X | 2.366 | X | 2.256 | X | 2.33 | 2.32 | 85.9 |
| 6 | X | 1.851 | X | 1.679 | X | 1.791 | 1.77 | 65.7 |
| 24 | X | 0.637 | X | 0.656 | X | 0.638 | 0.64 | 23.9 |

TABLE 35
SD 15194

| | | | | | | | | Conc. |
|---|---|---|---|---|---|---|---|---|
| Timepoint (h) | Tube 1 | | Tube 2 | | Tube 3 | | Mean | (% of T0) |
| 0 | X | 2.606 | X | 2.488 | X | 2.617 | 2.57 | 100.0 |
| 2 | X | 2.526 | X | 2.467 | X | 2.373 | 2.46 | 95.5 |
| 6 | X | 2.178 | X | 2.256 | X | 2.154 | 2.20 | 85.4 |
| 24 | X | 1.543 | X | 1.638 | X | 1.62 | 1.60 | 62.3 |

TABLE 36
SD 15193

| | | | | | | | | Conc. |
|---|---|---|---|---|---|---|---|---|
| Timepoint (h) | Tube 1 | | Tube 2 | | Tube 3 | | Mean | (% of T0) |
| 0 | X | 3.315 | X | 3.236 | X | 3.311 | 3.29 | 100.0 |
| 2 | X | 2.864 | X | 2.972 | X | 2.789 | 2.88 | 87.5 |
| 6 | X | 2.383 | X | 2.443 | X | 2.346 | 2.39 | 72.7 |
| 24 | X | 1.253 | X | 1.397 | X | 1.236 | 1.30 | 39.4 |

TABLE 37
SD 15196

| | | | | | | | | Conc. |
|---|---|---|---|---|---|---|---|---|
| Timepoint (h) | Tube 1 | | Tube 2 | | Tube 3 | | Mean | (% of T0) |
| 0 | X | 2.336 | X | 2.424 | X | 2.497 | 2.42 | 100.0 |
| 2 | X | 2.277 | X | 2.329 | X | 2.237 | 2.28 | 94.3 |
| 6 | X | 1.96 | X | 2.008 | X | 1.874 | 1.95 | 80.5 |
| 24 | X | 1.334 | X | 1.273 | X | 1.201 | 1.27 | 52.5 |

TABLE 38
SD 15805

| Timepoint | | | | Conc. |
|---|---|---|---|---|
| (h) | Tube 1 | Tube 2 | Mean | (% of T0) |
| 0.00 x | 2.583 x | 2.811 | 2.697 | 100.0 |
| 1.00 x | 2.223 x | 2.203 | 2.213 | 82.1 |
| 3.00 x | 2.125 x | 2.099 | 2.112 | 78.3 |
| 6.00 x | 1.429 x | 1.471 | 1.450 | 53.8 |

TABLE 39
SD 15807

| Timepoint | | | | Conc. |
|---|---|---|---|---|
| (h) | Tube 1 | Tube 2 | Mean | (% of T0) |
| 0.00 x | 2.149 x | 2.194 | 2.172 | 100.0 |
| 1.00 x | 1.726 x | 1.693 | 1.710 | 78.7 |
| 3.00 x | 1.321 x | 1.323 | 1.322 | 60.9 |
| 6.00 x | 0.803 x | 0.804 | 0.804 | 37.0 |

TABLE 40
SD 15806

| Timepoint | | | | Conc. |
|---|---|---|---|---|
| (h) | Tube 1 | Tube 2 | Mean | (% of T0) |
| 0.00 x | 3.000 x | 3.145 | 3.073 | 100.0 |
| 1.00 x | 2.773 x | 2.661 | 2.717 | 88.4 |
| 3.00 x | 2.604 x | 2.501 | 2.553 | 83.1 |
| 6.00 x | 2.258 x | 2.272 | 2.265 | 73.7 |

TABLE 41
SD 15808

| Timepoint | | | | Conc. |
|---|---|---|---|---|
| (h) | Tube 1 | Tube 2 | Mean | (% of T0) |
| 0.00 x | 3.223 x | 3.173 | 3.198 | 100.0 |
| 1.00 x | 2.668 x | 2.892 | 2.780 | 86.9 |
| 3.00 x | 2.398 x | 2.316 | 2.357 | 73.7 |
| 6.00 x | 2.018 x | 1.892 | 1.955 | 61.1 |

A number of embodiments have been described as disclosed herein. Nevertheless, it will be understood that various modifications may be made without departing from the spirit and scope of the embodiments as disclosed herein. Accordingly, other embodiments are within the scope of the following claims.

1. A compound comprising a peptide which comprises the cyclized amino acid sequence
-AA-AA1-AA2-AA3-AA4-AA5-AA6-AA7-AA8-AA9-AA10-
AA11-AA12-AA13-AA14-,
wherein the side-chains of any two of the amino acids of AA0, AA1, AA2, AA3, AA4, AA5, AA6, AA7, AA8, AA9, AA10, AA11, AA12, AA13 and AA14 (X1, X2) are connected covalently; and
wherein the amino acids of AA0, AA1, AA2, AA3, AA4, AA5, AA6, AA7, AA8, AA9, AA10, AA11, AA12, AA13 and AA14 which are not X1 and X2 are:
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA10 is Arg;
AA11 is Leu;
AA12 is Thr, Ser, Asp, Glu, Pro, Ala, or deleted;
AA13 is Glu, Lys, Pro, Ser, Ala, Asp, or deleted; and
AA14 is Pro or deleted.

2. The compound of claim 1 wherein X1 and X2 are separated by 2 or 3 amino acids.

3. The compound of claim 1 wherein AA5 is X1 and AA9 is X2, AA5 is X1 and AA8 is X2, AA9 is X1 and AA13 is X2, AA6 is X1 and AA9 is X2, AA9 is X1 and AA12 is X2, AA10 is X1 and AA13 is X2, AA6 is X1 and AA10 is X2 or AA4 is X1 and AA8 is X2.

4. The compound of claim 1 wherein the cyclized amino acid sequence is selected from the group consisting of
a.
-AA0-AA1-AA2-AA3-AA4-cyclo(X1-AA6-AA7-AA8-X2)-
Arg-Leu-AA12-AA13-AA14-,
b.
-AA0-AA1-AA2-AA3-AA4-cyclo(X1-AA6-AA7-X2)-AA9-
Arg-Leu-AA12-AA13-AA14-,
c.
-AA0-AA1-AA2-AA3-AA4-AA5-AA6-AA7-AA8-cyclo(X1-
Arg-Leu-AA12-X2)-AA14-,
d.
-AA0-AA1-AA2-AA3-AA4-AA5-cyclo(X1-AA7-AA8-X2)-
Arg-Leu-AA12-AA13-AA14-,
e.
-AA0-AA1-AA2-AA3-AA4-AA5-AA6-AA7-AA8-cyclo(X1-
Arg-Leu-X2)-AA13-AA14-,
f.
-AA0-AA1-AA2-AA3-AA4-AA5-AA6-AA7-AA8-AA9-cyclo
(X1-Leu-AA12-X2)-AA14-,
g.
-AA0-AA1-AA2-AA3-AA4-AA5-cyclo(X1-AA7-AA8-AA9-
X2)-Leu-AA12-AA13-AA14-,
and
h.
-AA0-AA1-AA2-AA3-cyclo(X1-AA5-AA6-AA7-X2)-AA9-
Arg-Leu-AA12-AA13-AA14-,
wherein the side-chains of X₁ and X₂ amino acid residues are connected covalently; and
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA12 is Thr, Ser, Asp, Glu, Pro, Ala, or deleted;
AA13 is Glu, Lys, Pro, Ser, Ala, Asp, or deleted; and
AA14 is Pro or deleted.

5. The compound of claim 1 wherein X₁ and X₂ are connected covalently via an amide, disulfide, thioether, ether, ester, thioester, thioamide, alkylene, alkenylene, alkynylene, and/or 1,2,3-triazole.

6. The compound of claim 1 wherein X₁ and X₂ are, independently, selected from the group consisting of Glu, DGlu, Asp, DAsp, Lys, DLys, hLys, DhLys, Orn, DOrn, Dab, DDab, Dap, DDap, Cys, DCys, hCys, DhCys, Pen, and DPen, with the proviso that when X₁ is Glu, DGlu, Asp, or DAsp, X₂ is Lys, DLys, hLys, DhLys, Orn, DOrn, Dab, DDab, Dap, or DDap;
when X1 is Lys, DLys, hLys, DhLys, Orn, DOrn, Dab, DDab, Dap, or DDap, X2 is Glu, DGlu, Asp, or DAsp; and
when X1 is Cys, DCys, hCys, DhCys, Pen, or DPen, X2 is Cys, DCys, hCys, DhCys, Pen, or DPen.

7. The compound of claim 1 wherein the cyclized amino acid sequence is selected from the group consisting of
-Ser-Arg-Leu-Glu-cyclo(Glu-Glu-Leu-Arg-Lys)-Arg-Leu-Thr-Glu-,
-Ser-Arg-Leu-Glu-cyclo(Asp-Glu-Leu-Arg-Lys)-Arg-Leu-Thr-Glu-,
-Ser-Arg-Leu-Glu-cyclo(Glu-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Glu)-,
-Ser-Arg-Leu-Glu-cyclo(Cys-Glu-Leu-Arg-Cys)-Arg-Leu-Thr-Glu-,
-Ser-Arg-Leu-Glu-cyclo(Asp-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Asp)-,
-Ser-Arg-Leu-Glu-cyclo(Glu-Glu-Leu-Arg-DLys)-Arg-Leu-Thr-Glu-,
-Pro-Ser-Arg-Leu-Glu-cyclo(Glu-Glu-Leu-Arg-Lys)-Arg-Leu-Thr-Glu-,
-Pro-Ser-Arg-Leu-Glu-cyclo(DGlu-Glu-Leu-Arg-Lys)-Arg-Leu-Thr-Glu-,
-Pro-Ser-Arg-Leu-Glu-cyclo(Glu-Glu-Leu-Arg-DLys)-Arg-Leu-Thr-Glu-,
-Pro-Ser-Arg-Leu-Glu-cyclo(Lys-Glu-Leu-Arg-Glu)-Arg-Leu-Thr-Glu-,
-Pro-Ser-Arg-Leu-cyclo(Glu-Glu-Glu-Leu-Lys)-Arg-Arg-Leu-Thr-Glu-,
-Pro-Ser-Arg-Leu-Glu-cyclo(Cys-Glu-Leu-DCys)-Arg-Arg-Leu-Thr-Glu-,
-Pro-Ser-Arg-Leu-Glu-Glu-Glu-Leu-Arg-cyclo(Cys-Arg-Leu-Thr-Cys)-,
-Pro-Ser-Arg-Leu-Glu-cyclo(Cys-Glu-Leu-Arg-Cys)-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-cyclo(Cys-Leu-Arg-Arg-Cys)-Leu-Thr-Glu-,
-Ser-Arg-Leu-Glu-Glu-Glu-Leu-Arg-cyclo(Cys-Arg-Leu-Thr-Cys)-,
-Ser-Arg-Leu-Glu-cyclo(Cys-Glu-Leu-Arg-Cys)-Arg-Leu-Thr-Glu-,
-Ser-Arg-Leu-Glu-cyclo(Cys-Glu-Leu-Cys)-Arg-Arg-Leu-Thr-Glu-,
-Ser-Arg-Leu-Glu-Glu-Glu-Leu-Arg-Arg-cyclo(Cys-Leu-Thr-Cys)-,
-Ser-Arg-Leu-Glu-Glu-cyclo(Cys-Leu-Arg-Arg-Cys)-Leu-Thr-Glu-,
-Ser-Arg-Leu-Glu-cyclo(DGlu-Glu-Leu-Arg-DLys)-Arg-Leu-Thr-Glu-,
-Ser-Arg-Leu-Glu-cyclo(DLys-Glu-Leu-Arg-Glu)-Arg-Leu-Thr-Glu-,
-Ser-Arg-Leu-Glu-cyclo(Lys-Glu-Leu-Arg-DGlu)-Arg-Leu-Thr-Glu-,
-Ser-Arg-Leu-Glu-cyclo(DLys-Glu-Leu-Arg-DGlu)-Arg-Leu-Thr-Glu-,
-Pro-Ser-Arg-Leu-Glu-cyclo(DGlu-Glu-Leu-Arg-DLys)-Arg-Leu-Thr-Glu-,
-Pro-Ser-Arg-Leu-Glu-cyclo(DLys-Glu-Leu-Arg-Glu)-Arg-Leu-Thr-Glu-,
-Pro-Ser-Arg-Leu-Glu-cyclo(Lys-Glu-Leu-Arg-DGlu)-Arg-Leu-Thr-Glu-,
-Pro-Ser-Arg-Leu-Glu-cyclo(DLys-Glu-Leu-Arg-DGlu)-Arg-Leu-Thr-Glu-,
-Ser-Arg-Leu-Glu-cyclo(DLys-Glu-Leu-Arg-Asp)-Arg-Leu-Thr-Glu-,
-Ser-Arg-Leu-Glu-cyclo(DAsp-Glu-Leu-Arg-DLys)-Arg-Leu-Thr-Glu-,
-Ser-Arg-Leu-Glu-cyclo(DAsp-Glu-Leu-Arg-Lys)-Arg-Leu-Thr-Glu-,
-Ser-Arg-Leu-Glu-cyclo(Asp-Glu-Leu-Arg-DLys)-Arg-Leu-Thr-Glu-,
-Ser-Arg-Leu-Glu-cyclo(Lys-Glu-Leu-Arg-DAsp)-Arg-Leu-Thr-Glu-,
-Ser-Arg-Leu-Glu-cyclo(DLys-Glu-Leu-Arg-DAsp)-Arg-Leu-Thr-Glu-,
-Pro-Ser-Arg-Leu-Glu-cyclo(DLys-Glu-Leu-Arg-Asp)-Arg-Leu-Thr-Glu-,
-Pro-Ser-Arg-Leu-Glu-cyclo(DAsp-Glu-Leu-Arg-DLys)-Arg-Leu-Thr-Glu-,
-Pro-Ser-Arg-Leu-Glu-cyclo(DAsp-Glu-Leu-Arg-Lys)-Arg-Leu-Thr-Glu-,
-Pro-Ser-Arg-Leu-Glu-cyclo(Asp-Glu-Leu-Arg-DLys)-Arg-Leu-Thr-Glu-,
-Pro-Ser-Arg-Leu-Glu-cyclo(Lys-Glu-Leu-Arg-DAsp)-Arg-Leu-Thr-Glu-,
-Pro-Ser-Arg-Leu-Glu-cyclo(DLys-Glu-Leu-Arg-DAsp)-Arg-Leu-Thr-Glu-,
-Ser-Arg-Leu-Glu-cyclo(DGlu-Glu-Leu-Lys)-Arg-Arg-Leu-Thr-Glu-,
-Ser-Arg-Leu-Glu-cyclo(Glu-Glu-Leu-DLys)-Arg-Arg-Leu-Thr-Glu-,
-Ser-Arg-Leu-Glu-cyclo(DGlu-Glu-Leu-DLys)-Arg-Arg-Leu-Thr-Glu-,
-Ser-Arg-Leu-Glu-cyclo(Lys-Glu-Leu-Glu)-Arg-Arg-Leu-Thr-Glu-,
-Ser-Arg-Leu-Glu-cyclo(Lys-Glu-Leu-DGlu)-Arg-Arg-Leu-Thr-Glu-,
-Ser-Arg-Leu-Glu-cyclo(DLys-Glu-Leu-DGlu)-Arg-Arg-Leu-Thr-Glu-,
-Ser-Arg-Leu-Glu-cyclo(Asp-Glu-Leu-Lys)-Arg-Arg-Leu-Thr-Glu-,
-Ser-Arg-Leu-Glu-cyclo(Asp-Glu-Leu-DLys)-Arg-Arg-Leu-Thr-Glu-,
-Ser-Arg-Leu-Glu-cyclo(Lys-Glu-Leu-Asp)-Arg-Arg-Leu-Thr-Glu-,
-Ser-Arg-Leu-Glu-cyclo(Lys-Glu-Leu-DAsp)-Arg-Arg-Leu-Thr-Glu-,
-Ser-Arg-Leu-Glu-cyclo(DAsp-Glu-Leu-Lys)-Arg-Arg-Leu-Thr-Glu-,
-Ser-Arg-Leu-Glu-cyclo(DAsp-Glu-Leu-DLys)-Arg-Arg-Leu-Thr-Glu-,
-Ser-Arg-Leu-Glu-cyclo(DLys-Glu-Leu-DAsp)-Arg-Arg-Leu-Thr-Glu-,
-Ser-Arg-Leu-Glu-cyclo(DLys-Glu-Leu-Asp)-Arg-Arg-Leu-Thr-Glu-,
-Pro-Ser-Arg-Leu-Glu-cyclo(Glu-Glu-Leu-DLys)-Arg-Arg-Leu-Thr-Glu-,
-Pro-Ser-Arg-Leu-Glu-cyclo(DGlu-Glu-Leu-DLys)-Arg-Arg-Leu-Thr-Glu-
-Pro-Ser-Arg-Leu-Glu-cyclo(Lys-Glu-Leu-Glu)-Arg-Arg-Leu-Thr-Glu-,
-Pro-Ser-Arg-Leu-Glu-cyclo(Lys-Glu-Leu-DGlu)-Arg-Arg-Leu-Thr-Glu-,
-Pro-Ser-Arg-Leu-Glu-cyclo(DLys-Glu-Leu-DGlu)-Arg-Arg-Leu-Thr-Glu-,
-Pro-Ser-Arg-Leu-Glu-cyclo(Asp-Glu-Leu-Lys)-Arg-Arg-Leu-Thr-Glu-,
-Pro-Ser-Arg-Leu-Glu-cyclo(Asp-Glu-Leu-DLys)-Arg-Arg-Leu-Thr-Glu-,
-Pro-Ser-Arg-Leu-Glu-cyclo(Lys-Glu-Leu-Asp)-Arg-Arg-Leu-Thr-Glu-,
-Pro-Ser-Arg-Leu-Glu-cyclo(Lys-Glu-Leu-DAsp)-Arg-Arg-Leu-Thr-Glu-,
-Pro-Ser-Arg-Leu-Glu-cyclo(DAsp-Glu-Leu-Lys)-Arg-Arg-Leu-Thr-Glu-,
-Pro-Ser-Arg-Leu-Glu-cyclo(DAsp-Glu-Leu-DLys)-Arg-Arg-Leu-Thr-Glu-,
-Pro-Ser-Arg-Leu-Glu-cyclo(DLys-Glu-Leu-DAsp)-Arg-Arg-Leu-Thr-Glu-,
-Pro-Ser-Arg-Leu-Glu-cyclo(DLys-Glu-Leu-Asp)-Arg-Arg-Leu-Thr-Glu-,
-Ser-Arg-Leu-Glu-Glu-cyclo(Asp-Leu-Arg-Lys)-Arg-Leu-Thr-Glu-,
-Ser-Arg-Leu-Glu-Glu-cyclo(Lys-Leu-Arg-Asp)-Arg-Leu-Thr-Glu-,
-Ser-Arg-Leu-Glu-Glu-cyclo(DAsp-Leu-Arg-Lys)-Arg-Leu-Thr-Glu-,
-Ser-Arg-Leu-Glu-Glu-cyclo(Lys-Leu-Arg-DAsp)-Arg-Leu-Thr-Glu-,
-Ser-Arg-Leu-Glu-Glu-cyclo(Asp-Leu-Arg-DLys)-Arg-Leu-Thr-Glu-,
-Ser-Arg-Leu-Glu-Glu-cyclo(DLys-Leu-Arg-Asp)-Arg-Leu-Thr-Glu-,
-Ser-Arg-Leu-Glu-Glu-cyclo(DLys-Leu-Arg-DAsp)-Arg-Leu-Thr-Glu-,
-Ser-Arg-Leu-Glu-Glu-cyclo(DAsp-Leu-Arg-DLys)-Arg-Leu-Thr-Glu-,
-Pro-Ser-Arg-Leu-Glu-Glu-cyclo(Asp-Leu-Arg-Lys)-Arg-Leu-Thr-Glu-,
-Pro-Ser-Arg-Leu-Glu-Glu-cyclo(Lys-Leu-Arg-Asp)-Arg-Leu-Thr-Glu-,
-Pro-Ser-Arg-Leu-Glu-Glu-cyclo(DAsp-Leu-Arg-Lys)-Arg-Leu-Thr-Glu-,
-Pro-Ser-Arg-Leu-Glu-Glu-cyclo(Lys-Leu-Arg-DAsp)-Arg-Leu-Thr-Glu-,
-Pro-Ser-Arg-Leu-Glu-Glu-cyclo(Asp-Leu-Arg-DLys)-Arg-Leu-Thr-Glu-,
-Pro-Ser-Arg-Leu-Glu-Glu-cyclo(DLys-Leu-Arg-Asp)-Arg-Leu-Thr-Glu-,
-Pro-Ser-Arg-Leu-Glu-Glu-cyclo(DLys-Leu-Arg-DAsp)-Arg-Leu-Thr-Glu-,
-Pro-Ser-Arg-Leu-Glu-Glu-cyclo(DAsp-Leu-Arg-DLys)-Arg-Leu-Thr-Glu-,
-Ser-Arg-Leu-Glu-cyclo(Cys-Glu-Leu-Cys)-Arg-Arg-Leu-Thr-Glu-,
-Ser-Arg-Leu-Glu-Glu-Glu-Leu-Arg-Arg-cyclo(Cys-Leu-Thr-Cys)-,
-Ser-Arg-Leu-Glu-Glu-Glu-Leu-Arg-cyclo(Cys-Arg-Leu-Cys)-Glu-,
-Ser-Arg-Leu-Glu-Glu-cyclo(Cys-Leu-Arg-Cys)-Arg-Leu-Thr-Glu-,
-Pro-Ser-Arg-Leu-Glu-cyclo(Cys-Glu-Leu-Cys)-Arg-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-Glu-Leu-Arg-cyclo(Cys-Arg-Leu-Cys)-Glu-,
-Pro-Ser-Arg-Leu-Glu-Glu-cyclo(Cys-Leu-Arg-Cys)-Arg-Leu-Thr-Glu-,
-Ser-Arg-Leu-Glu-cyclo(DCys-Glu-Leu-DCys)-Arg-Arg-Leu-Thr-Glu-,
-Ser-Arg-Leu-Glu-cyclo(DCys-Glu-Leu-Cys)-Arg-Arg-Leu-Thr-Glu-,
-Ser-Arg-Leu-Glu-cyclo(hCys-Glu-Leu-Cys)-Arg-Arg-Leu-Thr-Glu-,
-Ser-Arg-Leu-Glu-cyclo(hCys-Glu-Leu-hCys)-Arg-Arg-Leu-Thr-Glu-,
-Ser-Arg-Leu-Glu-cyclo(Cys-Glu-Leu-hCys)-Arg-Arg-Leu-Thr-Glu-,
-Ser-Arg-Leu-Glu-cyclo(hCys-Glu-Leu-DCys)-Arg-Arg-Leu-Thr-Glu-,
-Ser-Arg-Leu-Glu-cyclo(DCys-Glu-Leu-hCys)-Arg-Arg-Leu-Thr-Glu-,
-Ser-Arg-Leu-Glu-cyclo(hCys-Glu-Leu-DhCys)-Arg-Arg-Leu-Thr-Glu-,
-Ser-Arg-Leu-Glu-cyclo(DhCys-Glu-Leu-DhCys)-Arg-Arg-Leu-Thr-Glu-,
-Ser-Arg-Leu-Glu-cyclo(Cys-Glu-Leu-DhCys)-Arg-Arg-Leu-Thr-Glu-,
-Ser-Arg-Leu-Glu-cyclo(DCys-Glu-Leu-DhCys)-Arg-Arg-Leu-Thr-Glu-,
-Ser-Arg-Leu-Glu-cyclo(DhCys-Glu-Leu-Cys)-Arg-Arg-Leu-Thr-Glu-,
-Ser-Arg-Leu-Glu-cyclo(DhCys-Glu-Leu-DCys)-Arg-Arg-Leu-Thr-Glu-,
-Pro-Ser-Arg-Leu-Glu-cyclo(DCys-Glu-Leu-DCys)-Arg-Arg-Leu-Thr-Glu-,
-Pro-Ser-Arg-Leu-Glu-cyclo(DCys-Glu-Leu-Cys)-Arg-Arg-Leu-Thr-Glu-,
-Pro-Ser-Arg-Leu-Glu-cyclo(hCys-Glu-Leu-Cys)-Arg-Arg-Leu-Thr-Glu-,
-Pro-Ser-Arg-Leu-Glu-cyclo(hCys-Glu-Leu-hCys)-Arg-Arg-Leu-Thr-Glu-,
-Pro-Ser-Arg-Leu-Glu-cyclo(Cys-Glu-Leu-hCys)-Arg-Arg-Leu-Thr-Glu-,
-Pro-Ser-Arg-Leu-Glu-cyclo(hCys-Glu-Leu-DCys)-Arg-Arg-Leu-Thr-Glu-,
-Pro-Ser-Arg-Leu-Glu-cyclo(DCys-Glu-Leu-hCys)-Arg-Arg-Leu-Thr-Glu-,
-Pro-Ser-Arg-Leu-Glu-cyclo(hCys-Glu-Leu-DhCys)-Arg-Arg-Leu-Thr-Glu-,
-Pro-Ser-Arg-Leu-Glu-cyclo(DhCys-Glu-Leu-DhCys)-Arg-Arg-Leu-Thr-Glu-,
-Pro-Ser-Arg-Leu-Glu-cyclo(Cys-Glu-Leu-DhCys)-Arg-Arg-Leu-Thr-Glu-,
-Pro-Ser-Arg-Leu-Glu-cyclo(DCys-Glu-Leu-DhCys)-Arg-Arg-Leu-Thr-Glu-,
-Pro-Ser-Arg-Leu-Glu-cyclo(DhCys-Glu-Leu-Cys)-Arg-Arg-Leu-Thr-Glu-,
-Pro-Ser-Arg-Leu-Glu-cyclo(DhCys-Glu-Leu-DCys)-Arg-Arg-Leu-Thr-Glu-,
-Ser-Arg-Leu-Glu-Glu-Glu-Leu-Arg-Arg-cyclo(Cys-Leu-Thr-DCys)-,
-Ser-Arg-Leu-Glu-Glu-Glu-Leu-Arg-Arg-cyclo(DCys-Leu-Thr-Cys)-,
-Ser-Arg-Leu-Glu-Glu-Glu-Leu-Arg-Arg-cyclo(DCys-Leu-Thr-DCys)-,
-Ser-Arg-Leu-Glu-Glu-Glu-Leu-Arg-Arg-cyclo(hCys-Leu-Thr-Cys)-,
-Ser-Arg-Leu-Glu-Glu-Glu-Leu-Arg-Arg-cyclo(hCys-Leu-Thr-DCys)-,
-Ser-Arg-Leu-Glu-Glu-Glu-Leu-Arg-Arg-cyclo(hCys-Leu-Thr-hCys)-,
-Ser-Arg-Leu-Glu-Glu-Glu-Leu-Arg-Arg-cyclo(Cys-Leu-Thr-hCys)-,
-Ser-Arg-Leu-Glu-Glu-Glu-Leu-Arg-Arg-cyclo(DCys-Leu-Thr-hCys)-,
-Ser-Arg-Leu-Glu-Glu-Glu-Leu-Arg-Arg-cyclo(Cys-Leu-Thr-hCys)-,
-Ser-Arg-Leu-Glu-Glu-Glu-Leu-Arg-Arg-cyclo(DhCys-Leu-Thr-Cys)-,
-Ser-Arg-Leu-Glu-Glu-Glu-Leu-Arg-Arg-cyclo(DhCys-Leu-Thr-DCys)-,
-Ser-Arg-Leu-Glu-Glu-Glu-Leu-Arg-Arg-cyclo(DhCys-Leu-Thr-hCys)-,
-Ser-Arg-Leu-Glu-Glu-Glu-Leu-Arg-Arg-cyclo(Cys-Leu-Thr-DhCys)-,
-Ser-Arg-Leu-Glu-Glu-Glu-Leu-Arg-Arg-cyclo(DCys-Leu-Thr-DhCys)-,
-Ser-Arg-Leu-Glu-Glu-Glu-Leu-Arg-Arg-cyclo(Cys-Leu-Thr-DhCys)-,
-Ser-Arg-Leu-Glu-Glu-Glu-Leu-Arg-Arg-cyclo(Pen-Leu-Thr-Pen)-,
-Ser-Arg-Leu-Glu-Glu-Glu-Leu-Arg-Arg-cyclo(Pen-Leu-Thr-DPen)-,
-Ser-Arg-Leu-Glu-Glu-Glu-Leu-Arg-Arg-cyclo(DPen-Leu-Thr-Pen)-,
-Ser-Arg-Leu-Glu-Glu-Glu-Leu-Arg-Arg-cyclo(DPen-Leu-Thr-DPen)-,
-Ser-Arg-Leu-Glu-Glu-Glu-Leu-Arg-cyclo(DCys-Arg-Leu-Cys)-Glu-,
-Ser-Arg-Leu-Glu-Glu-Glu-Leu-Arg-cyclo(DCys-Arg-Leu-DCys)-Glu-,
-Ser-Arg-Leu-Glu-Glu-Glu-Leu-Arg-cyclo(Cys-Arg-Leu-DCys)-Glu-,
-Ser-Arg-Leu-Glu-Glu-Glu-Leu-Arg-cyclo(hCys-Arg-Leu-Cys)-Glu-,
-Ser-Arg-Leu-Glu-Glu-Glu-Leu-Arg-cyclo(hCys-Arg-Leu-DCys)-Glu-,
-Ser-Arg-Leu-Glu-Glu-Glu-Leu-Arg-cyclo(Cys-Arg-Leu-hCys)-Glu-,
-Ser-Arg-Leu-Glu-Glu-Glu-Leu-Arg-cyclo(hCys-Arg-Leu-hCys)-Glu-,
-Ser-Arg-Leu-Glu-Glu-Glu-Leu-Arg-cyclo(hCys-Arg-Leu-DhCys)-Glu-,
-Ser-Arg-Leu-Glu-Glu-Glu-Leu-Arg-cyclo(DhCys-Arg-Leu-hCys)-Glu-,
-Pro-Ser-Arg-Leu-Glu-Glu-Glu-Leu-Arg-Arg-cyclo(Cys-Leu-Thr-DCys)-,
-Pro-Ser-Arg-Leu-Glu-Glu-Glu-Leu-Arg-Arg-cyclo(DCys-Leu-Thr-Cys)-,
-Pro-Ser-Arg-Leu-Glu-Glu-Glu-Leu-Arg-Arg-cyclo(DCys-Leu-Thr-DCys)-,
-Pro-Ser-Arg-Leu-Glu-Glu-Glu-Leu-Arg-Arg-cyclo(hCys-Leu-Thr-Cys)-,
-Pro-Ser-Arg-Leu-Glu-Glu-Glu-Leu-Arg-Arg-cyclo(hCys-Leu-Thr-DCys)-,
-Pro-Ser-Arg-Leu-Glu-Glu-Glu-Leu-Arg-Arg-cyclo(hCys-Leu-Thr-hCys)-,
-Pro-Ser-Arg-Leu-Glu-Glu-Glu-Leu-Arg-Arg-cyclo(Cys-Leu-Thr-hCys)-,
-Pro-Ser-Arg-Leu-Glu-Glu-Glu-Leu-Arg-Arg-cyclo(DCys-Leu-Thr-hCys)-,
-Pro-Ser-Arg-Leu-Glu-Glu-Glu-Leu-Arg-Arg-cyclo(Cys-Leu-Thr-hCys)-,
-Pro-Ser-Arg-Leu-Glu-Glu-Glu-Leu-Arg-Arg-cyclo(DhCys-Leu-Thr-Cys)-,
-Pro-Ser-Arg-Leu-Glu-Glu-Glu-Leu-Arg-Arg-cyclo(DhCys-Leu-Thr-DCys)-,
-Pro-Ser-Arg-Leu-Glu-Glu-Glu-Leu-Arg-Arg-cyclo(DhCys-Leu-Thr-hCys)-,
-Pro-Ser-Arg-Leu-Glu-Glu-Glu-Leu-Arg-Arg-cyclo(Cys-Leu-Thr-DhCys)-,
-Pro-Ser-Arg-Leu-Glu-Glu-Glu-Leu-Arg-Arg-cyclo(DCys-Leu-Thr-DhCys)-,
-Pro-Ser-Arg-Leu-Glu-Glu-Glu-Leu-Arg-Arg-cyclo(Cys-Leu-Thr-DhCys)-,
-Pro-Ser-Arg-Leu-Glu-Glu-Glu-Leu-Arg-Arg-cyclo(Pen-Leu-Thr-Pen)-,
-Pro-Ser-Arg-Leu-Glu-Glu-Glu-Leu-Arg-Arg-cyclo(Pen-Leu-Thr-DPen)-,
-Pro-Ser-Arg-Leu-Glu-Glu-Glu-Leu-Arg-Arg-cyclo(DPen-Leu-Thr-Pen)-,
-Pro-Ser-Arg-Leu-Glu-Glu-Glu-Leu-Arg-Arg-cyclo(DPen-Leu-Thr-DPen)-,
-Pro-Ser-Arg-Leu-Glu-Glu-Glu-Leu-Arg-cyclo(DCys-Arg-Leu-Cys)-Glu-,
-Pro-Ser-Arg-Leu-Glu-Glu-Glu-Leu-Arg-cyclo(DCys-Arg-Leu-DCys)-Glu-,
-Pro-Ser-Arg-Leu-Glu-Glu-Glu-Leu-Arg-cyclo(Cys-Arg-Leu-DCys)-Glu-,
-Pro-Ser-Arg-Leu-Glu-Glu-Glu-Leu-Arg-cyclo(hCys-Arg-Leu-Cys)-Glu-,
-Pro-Ser-Arg-Leu-Glu-Glu-Glu-Leu-Arg-cyclo(hCys-Arg-Leu-DCys)-Glu-,
-Pro-Ser-Arg-Leu-Glu-Glu-Glu-Leu-Arg-cyclo(Cys-Arg-Leu-hCys)-Glu-,
-Pro-Ser-Arg-Leu-Glu-Glu-Glu-Leu-Arg-cyclo(hCys-Arg-Leu-hCys)-Glu-,
-Pro-Ser-Arg-Leu-Glu-Glu-Glu-Leu-Arg-cyclo(hCys-Arg-Leu-DhCys)-Glu-,
-Pro-Ser-Arg-Leu-Glu-Glu-Glu-Leu-Arg-cyclo(DhCys-Arg-Leu-hCys)-Glu-,
-Ser-Arg-Leu-Glu-Glu-Glu-Leu-Arg-cyclo(Lys-Arg-Leu-Asp)-Glu-,
-Ser-Arg-Leu-Glu-Glu-Glu-Leu-Arg-cyclo(Asp-Arg-Leu-Lys)-Glu-,
-Ser-Arg-Leu-Glu-Glu-Glu-Leu-Arg-cyclo(Lys-Arg-Leu-Glu)-Glu-,
-Ser-Arg-Leu-Glu-Glu-Glu-Leu-Arg-cyclo(Glu-Arg-Leu-Lys)-Glu-,
-Pro-Ser-Arg-Leu-Glu-Glu-Glu-Leu-Arg-cyclo(Lys-Arg-Leu-Asp)-Glu-,
-Pro-Ser-Arg-Leu-Glu-Glu-Glu-Leu-Arg-cyclo(Asp-Arg-Leu-Lys)-Glu-,
-Pro-Ser-Arg-Leu-Glu-Glu-Glu-Leu-Arg-cyclo(Lys-Arg-Leu-Glu)-Glu-,
-Pro-Ser-Arg-Leu-Glu-Glu-Glu-Leu-Arg-cyclo(Glu-Arg-Leu-Lys)-Glu-,
-Pro-Ser-Arg-Leu-Glu-Glu-Glu-Leu-Arg-cyclo(Cys-Arg-Leu-Thr-Cys)-,
-Pro-Ser-Arg-Leu-Glu-cyclo(Cys-Glu-Leu-Arg-Cys)-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-cyclo(Cys-Leu-Arg-Arg-Cys)-Leu-Thr-Glu-,
-Ser-Arg-Leu-Glu-cyclo(DGlu-Glu-Leu-Arg-DLys)-Arg-Leu-Thr-Glu-Pro-,
-Ser-Arg-Leu-Glu-cyclo(DLys-Glu-Leu-Arg-Glu)-Arg-Leu-Thr-Glu-Pro-,
-Ser-Arg-Leu-Glu-cyclo(Lys-Glu-Leu-Arg-DGlu)-Arg-Leu-Thr-Glu-Pro-,
-Ser-Arg-Leu-Glu-cyclo(DLys-Glu-Leu-Arg-DGlu)-Arg-Leu-Thr-Glu-Pro-,
-Pro-Ser-Arg-Leu-Glu-cyclo(DGlu-Glu-Leu-Arg-DLys)-Arg-Leu-Thr-Glu-Pro-,
-Pro-Ser-Arg-Leu-Glu-cyclo(DLys-Glu-Leu-Arg-Glu)-Arg-Leu-Thr-Glu-Pro-,
-Pro-Ser-Arg-Leu-Glu-cyclo(Lys-Glu-Leu-Arg-DGlu)-Arg-Leu-Thr-Glu-Pro-,
-Pro-Ser-Arg-Leu-Glu-cyclo(DLys-Glu-Leu-Arg-DGlu)-Arg-Leu-Thr-Glu-Pro-,
-Ser-Arg-Leu-Glu-cyclo(DLys-Glu-Leu-Arg-Asp)-Arg-Leu-Thr-Glu-Pro-,
-Ser-Arg-Leu-Glu-cyclo(DAsp-Glu-Leu-Arg-DLys)-Arg-Leu-Thr-Glu-Pro-,
-Ser-Arg-Leu-Glu-cyclo(DAsp-Glu-Leu-Arg-Lys)-Arg-Leu-Thr-Glu-Pro-,
-Ser-Arg-Leu-Glu-cyclo(Asp-Glu-Leu-Arg-DLys)-Arg-Leu-Thr-Glu-Pro-,
-Ser-Arg-Leu-Glu-cyclo(Lys-Glu-Leu-Arg-DAsp)-Arg-Leu-Thr-Glu-Pro-,
-Ser-Arg-Leu-Glu-cyclo(DLys-Glu-Leu-Arg-DAsp)-Arg-Leu-Thr-Glu-Pro-,
-Pro-Ser-Arg-Leu-Glu-cyclo(DLys-Glu-Leu-Arg-Asp)-Arg-Leu-Thr-Glu-Pro-,
-Pro-Ser-Arg-Leu-Glu-cyclo(DAsp-Glu-Leu-Arg-DLys)-Arg-Leu-Thr-Glu-Pro-,
-Pro-Ser-Arg-Leu-Glu-cyclo(DAsp-Glu-Leu-Arg-Lys)-Arg-Leu-Thr-Glu-Pro-,
-Pro-Ser-Arg-Leu-Glu-cyclo(Asp-Glu-Leu-Arg-DLys)-Arg-Leu-Thr-Glu-Pro-,
-Pro-Ser-Arg-Leu-Glu-cyclo(Lys-Glu-Leu-Arg-DAsp)-Arg-Leu-Thr-Glu-Pro-,
-Pro-Ser-Arg-Leu-Glu-cyclo(DLys-Glu-Leu-Arg-DAsp)-Arg-Leu-Thr-Glu-Pro-,
-Ser-Arg-Leu-Glu-cyclo(DGlu-Glu-Leu-Lys)-Arg-Arg-Leu-Thr-Glu-Pro-,
-Ser-Arg-Leu-Glu-cyclo(Glu-Glu-Leu-DLys)-Arg-Arg-Leu-Thr-Glu-Pro-,
-Ser-Arg-Leu-Glu-cyclo(DGlu-Glu-Leu-DLys)-Arg-Arg-Leu-Thr-Glu-Pro-,
-Ser-Arg-Leu-Glu-cyclo(Lys-Glu-Leu-Glu)-Arg-Arg-Leu-Thr-Glu-Pro-,
-Ser-Arg-Leu-Glu-cyclo(Lys-Glu-Leu-DGlu)-Arg-Arg-Leu-Thr-Glu-Pro-,
-Ser-Arg-Leu-Glu-cyclo(DLys-Glu-Leu-DGlu)-Arg-Arg-Leu-Thr-Glu-Pro-,
-Ser-Arg-Leu-Glu-cyclo(Asp-Glu-Leu-Lys)-Arg-Arg-Leu-Thr-Glu-Pro-,
-Ser-Arg-Leu-Glu-cyclo(Asp-Glu-Leu-DLys)-Arg-Arg-Leu-Thr-Glu-Pro-,
-Ser-Arg-Leu-Glu-cyclo(Lys-Glu-Leu-Asp)-Arg-Arg-Leu-Thr-Glu-Pro-,
-Ser-Arg-Leu-Glu-cyclo(Lys-Glu-Leu-DAsp)-Arg-Arg-Leu-Thr-Glu-Pro-,
-Ser-Arg-Leu-Glu-cyclo(DAsp-Glu-Leu-Lys)-Arg-Arg-Leu-Thr-Glu-Pro-,
-Ser-Arg-Leu-Glu-cyclo(DAsp-Glu-Leu-DLys)-Arg-Arg-Leu-Thr-Glu-Pro-,
-Ser-Arg-Leu-Glu-cyclo(DLys-Glu-Leu-DAsp)-Arg-Arg-Leu-Thr-Glu-Pro-,
-Ser-Arg-Leu-Glu-cyclo(DLys-Glu-Leu-Asp)-Arg-Arg-Leu-Thr-Glu-Pro-,
-Pro-Ser-Arg-Leu-Glu-cyclo(Glu-Glu-Leu-DLys)-Arg-Arg-Leu-Thr-Glu-Pro-,
-Pro-Ser-Arg-Leu-Glu-cyclo(DGlu-Glu-Leu-DLys)-Arg-Arg-Leu-Thr-Glu-Pro-,
-Pro-Ser-Arg-Leu-Glu-cyclo(Lys-Glu-Leu-Glu)-Arg-Arg-Leu-Thr-Glu-Pro-,
-Pro-Ser-Arg-Leu-Glu-cyclo(Lys-Glu-Leu-DGlu)-Arg-Arg-Leu-Thr-Glu-Pro-,
-Pro-Ser-Arg-Leu-Glu-cyclo(DLys-Glu-Leu-DGlu)-Arg-Arg-Leu-Thr-Glu-Pro-,
-Pro-Ser-Arg-Leu-Glu-cyclo(Asp-Glu-Leu-Lys)-Arg-Arg-Leu-Thr-Glu-Pro-,
-Pro-Ser-Arg-Leu-Glu-cyclo(Asp-Glu-Leu-DLys)-Arg-Arg-Leu-Thr-Glu-Pro-,
-Pro-Ser-Arg-Leu-Glu-cyclo(Lys-Glu-Leu-Asp)-Arg-Arg-Leu-Thr-Glu-Pro-,
-Pro-Ser-Arg-Leu-Glu-cyclo(Lys-Glu-Leu-DAsp)-Arg-Arg-Leu-Thr-Glu-Pro-,
-Pro-Ser-Arg-Leu-Glu-cyclo(DAsp-Glu-Leu-Lys)-Arg-Arg-Leu-Thr-Glu-Pro-,
-Pro-Ser-Arg-Leu-Glu-cyclo(DAsp-Glu-Leu-DLys)-Arg-Arg-Leu-Thr-Glu-Pro-,
-Pro-Ser-Arg-Leu-Glu-cyclo(DLys-Glu-Leu-DAsp)-Arg-Arg-Leu-Thr-Glu-Pro-,
-Pro-Ser-Arg-Leu-Glu-cyclo(DLys-Glu-Leu-Asp)-Arg-Arg-Leu-Thr-Glu-Pro-,
-Ser-Arg-Leu-Glu-Glu-cyclo(Asp-Leu-Arg-Lys)-Arg-Leu-Thr-Glu-Pro-,
-Ser-Arg-Leu-Glu-Glu-cyclo(Lys-Leu-Arg-Asp)-Arg-Leu-Thr-Glu-Pro-,
-Ser-Arg-Leu-Glu-Glu-cyclo(DAsp-Leu-Arg-Lys)-Arg-Leu-Thr-Glu-Pro-,
-Ser-Arg-Leu-Glu-Glu-cyclo(Lys-Leu-Arg-DAsp)-Arg-Leu-Thr-Glu-Pro-,
-Ser-Arg-Leu-Glu-Glu-cyclo(Asp-Leu-Arg-DLys)-Arg-Leu-Thr-Glu-Pro-,
-Ser-Arg-Leu-Glu-Glu-cyclo(DLys-Leu-Arg-Asp)-Arg-Leu-Thr-Glu-Pro-,
-Ser-Arg-Leu-Glu-Glu-cyclo(DLys-Leu-Arg-DAsp)-Arg-Leu-Thr-Glu-Pro-,
-Ser-Arg-Leu-Glu-Glu-cyclo(DAsp-Leu-Arg-DLys)-Arg-Leu-Thr-Glu-Pro-,
-Pro-Ser-Arg-Leu-Glu-Glu-cyclo(Asp-Leu-Arg-Lys)-Arg-Leu-Thr-Glu-Pro-,
-Pro-Ser-Arg-Leu-Glu-Glu-cyclo(Lys-Leu-Arg-Asp)-Arg-Leu-Thr-Glu-Pro-,
-Pro-Ser-Arg-Leu-Glu-Glu-cyclo(DAsp-Leu-Arg-Lys)-Arg-Leu-Thr-Glu-Pro-,
-Pro-Ser-Arg-Leu-Glu-Glu-cyclo(Lys-Leu-Arg-DAsp)-Arg-Leu-Thr-Glu-Pro-,
-Pro-Ser-Arg-Leu-Glu-Glu-cyclo(Asp-Leu-Arg-DLys)-Arg-Leu-Thr-Glu-Pro-,
-Pro-Ser-Arg-Leu-Glu-Glu-cyclo(DLys-Leu-Arg-Asp)-Arg-Leu-Thr-Glu-Pro-,
-Pro-Ser-Arg-Leu-Glu-Glu-cyclo(DLys-Leu-Arg-DAsp)-Arg-Leu-Thr-Glu-Pro-,
-Pro-Ser-Arg-Leu-Glu-Glu-cyclo(DAsp-Leu-Arg-DLys)-Arg-Leu-Thr-Glu-Pro-,
-Ser-Arg-Leu-Glu-cyclo(Cys-Glu-Leu-Cys)-Arg-Arg-Leu-Thr-Glu-Pro-,
-Ser-Arg-Leu-Glu-Glu-Glu-Leu-Arg-Arg-cyclo(Cys-Leu-Thr-Cys)-Pro-,
-Ser-Arg-Leu-Glu-Glu-Glu-Leu-Arg-cyclo(Cys-Arg-Leu-Cys)-Glu-Pro-,
-Ser-Arg-Leu-Glu-Glu-cyclo(Cys-Leu-Arg-Cys)-Arg-Leu-Thr-Glu-Pro-,
-Pro-Ser-Arg-Leu-Glu-cyclo(Cys-Glu-Leu-Cys)-Arg-Arg-Leu-Thr-Glu-Pro-,
-Pro-Ser-Arg-Leu-Glu-Glu-Glu-Leu-Arg-Arg-cyclo(Cys-Leu-Thr-Cys)-Pro-,
-Pro-Ser-Arg-Leu-Glu-Glu-Glu-Leu-Arg-cyclo(Cys-Arg-Leu-Cys)-Glu-Pro-,
-Pro-Ser-Arg-Leu-Glu-Glu-cyclo(Cys-Leu-Arg-Cys)-Arg-Leu-Thr-Glu-Pro-,
-Ser-Arg-Leu-Glu-cyclo(DCys-Glu-Leu-DCys)-Arg-Arg-Leu-Thr-Glu-Pro-,
-Ser-Arg-Leu-Glu-cyclo(DCys-Glu-Leu-Cys)-Arg-Arg-Leu-Thr-Glu-Pro-,
-Ser-Arg-Leu-Glu-cyclo(hCys-Glu-Leu-Cys)-Arg-Arg-Leu-Thr-Glu-Pro-,
-Ser-Arg-Leu-Glu-cyclo(hCys-Glu-Leu-hCys)-Arg-Arg-Leu-Thr-Glu-Pro-,
-Ser-Arg-Leu-Glu-cyclo(Cys-Glu-Leu-hCys)-Arg-Arg-Leu-Thr-Glu-Pro-,
-Ser-Arg-Leu-Glu-cyclo(hCys-Glu-Leu-DCys)-Arg-Arg-Leu-Thr-Glu-Pro-,
-Ser-Arg-Leu-Glu-cyclo(DCys-Glu-Leu-hCys)-Arg-Arg-Leu-Thr-Glu-Pro-,
-Ser-Arg-Leu-Glu-cyclo(hCys-Glu-Leu-DhCys)-Arg-Arg-Leu-Thr-Glu-Pro-,
-Ser-Arg-Leu-Glu-cyclo(DhCys-Glu-Leu-DhCys)-Arg-Arg-Leu-Thr-Glu-Pro-,
-Ser-Arg-Leu-Glu-cyclo(Cys-Glu-Leu-DhCys)-Arg-Arg-Leu-Thr-Glu-Pro-,
-Ser-Arg-Leu-Glu-cyclo(DCys-Glu-Leu-DhCys)-Arg-Arg-Leu-Thr-Glu-Pro-,
-Ser-Arg-Leu-Glu-cyclo(DhCys-Glu-Leu-Cys)-Arg-Arg-Leu-Thr-Glu-Pro-,
-Ser-Arg-Leu-Glu-cyclo(DhCys-Glu-Leu-DCys)-Arg-Arg-Leu-Thr-Glu-Pro-,
-Pro-Ser-Arg-Leu-Glu-cyclo(DCys-Glu-Leu-DCys)-Arg-Arg-Leu-Thr-Glu-Pro-,
-Pro-Ser-Arg-Leu-Glu-cyclo(DCys-Glu-Leu-Cys)-Arg-Arg-Leu-Thr-Glu-Pro-,
-Pro-Ser-Arg-Leu-Glu-cyclo(hCys-Glu-Leu-Cys)-Arg-Arg-Leu-Thr-Glu-Pro-,
-Pro-Ser-Arg-Leu-Glu-cyclo(hCys-Glu-Leu-hCys)-Arg-Arg-Leu-Thr-Glu-Pro-,
-Pro-Ser-Arg-Leu-Glu-cyclo(Cys-Glu-Leu-hCys)-Arg-Arg-Leu-Thr-Glu-Pro-,
-Pro-Ser-Arg-Leu-Glu-cyclo(hCys-Glu-Leu-DCys)-Arg-Arg-Leu-Thr-Glu-Pro-,
-Pro-Ser-Arg-Leu-Glu-cyclo(DCys-Glu-Leu-hCys)-Arg-Arg-Leu-Thr-Glu-Pro-,
-Pro-Ser-Arg-Leu-Glu-cyclo(hCys-Glu-Leu-DhCys)-Arg-Arg-Leu-Thr-Glu-Pro-,
-Pro-Ser-Arg-Leu-Glu-cyclo(DhCys-Glu-Leu-DhCys)-Arg-Arg-Leu-Thr-Glu-Pro-,
-Pro-Ser-Arg-Leu-Glu-cyclo(Cys-Glu-Leu-DhCys)-Arg-Arg-Leu-Thr-Glu-Pro-,
-Pro-Ser-Arg-Leu-Glu-cyclo(DCys-Glu-Leu-DhCys)-Arg-Arg-Leu-Thr-Glu-Pro-,
-Pro-Ser-Arg-Leu-Glu-cyclo(DhCys-Glu-Leu-Cys)-Arg-Arg-Leu-Thr-Glu-Pro-,
-Pro-Ser-Arg-Leu-Glu-cyclo(DhCys-Glu-Leu-DCys)-Arg-Arg-Leu-Thr-Glu-Pro-,
-Ser-Arg-Leu-Glu-Glu-Glu-Leu-Arg-Arg-cyclo(Cys-Leu-Thr-DCys)-Pro-,
-Ser-Arg-Leu-Glu-Glu-Glu-Leu-Arg-Arg-cyclo(DCys-Leu-Thr-Cys)-Pro-,
-Ser-Arg-Leu-Glu-Glu-Glu-Leu-Arg-Arg-cyclo(DCys-Leu-Thr-DCys)-Pro-,
-Ser-Arg-Leu-Glu-Glu-Glu-Leu-Arg-Arg-cyclo(hCys-Leu-Thr-Cys)-Pro-,
-Ser-Arg-Leu-Glu-Glu-Glu-Leu-Arg-Arg-cyclo(hCys-Leu-Thr-DCys)-Pro-,
-Ser-Arg-Leu-Glu-Glu-Glu-Leu-Arg-Arg-cyclo(hCys-Leu-Thr-hCys)-Pro-,
-Ser-Arg-Leu-Glu-Glu-Glu-Leu-Arg-Arg-cyclo(Cys-Leu-Thr-hCys)-Pro-,
-Ser-Arg-Leu-Glu-Glu-Glu-Leu-Arg-Arg-cyclo(DCys-Leu-Thr-hCys)-Pro-,
-Ser-Arg-Leu-Glu-Glu-Glu-Leu-Arg-Arg-cyclo(Cys-Leu-Thr-hCys)-Pro-,
-Ser-Arg-Leu-Glu-Glu-Glu-Leu-Arg-Arg-cyclo(DhCys-Leu-Thr-Cys)-Pro-,
-Ser-Arg-Leu-Glu-Glu-Glu-Leu-Arg-Arg-cyclo(DhCys-Leu-Thr-DCys)-Pro-,
-Ser-Arg-Leu-Glu-Glu-Glu-Leu-Arg-Arg-cyclo(DhCys-Leu-Thr-hCys)-Pro-,
-Ser-Arg-Leu-Glu-Glu-Glu-Leu-Arg-Arg-cyclo(Cys-Leu-Thr-DhCys)-Pro-,
-Ser-Arg-Leu-Glu-Glu-Glu-Leu-Arg-Arg-cyclo(DCys-Leu-Thr-DhCys)-Pro-,
-Ser-Arg-Leu-Glu-Glu-Glu-Leu-Arg-Arg-cyclo(Cys-Leu-Thr-DhCys)-Pro-,
-Ser-Arg-Leu-Glu-Glu-Glu-Leu-Arg-Arg-cyclo(Pen-Leu-Thr-Pen)-Pro-,
-Ser-Arg-Leu-Glu-Glu-Glu-Leu-Arg-Arg-cyclo(Pen-Leu-Thr-DPen)-Pro-,
-Ser-Arg-Leu-Glu-Glu-Glu-Leu-Arg-Arg-cyclo(DPen-Leu-Thr-Pen)-Pro-,
-Ser-Arg-Leu-Glu-Glu-Glu-Leu-Arg-Arg-cyclo(DPen-Leu-Thr-DPen)-Pro-,
-Ser-Arg-Leu-Glu-Glu-Glu-Leu-Arg-cyclo(DCys-Arg-Leu-Cys)-Glu-Pro-,
-Ser-Arg-Leu-Glu-Glu-Glu-Leu-Arg-cyclo(DCys-Arg-Leu-DCys)-Glu-Pro-,
-Ser-Arg-Leu-Glu-Glu-Glu-Leu-Arg-cyclo(Cys-Arg-Leu-DCys)-Glu-Pro-,
-Ser-Arg-Leu-Glu-Glu-Glu-Leu-Arg-cyclo(hCys-Arg-Leu-Cys)-Glu-Pro-,
-Ser-Arg-Leu-Glu-Glu-Glu-Leu-Arg-cyclo(hCys-Arg-Leu-DCys)-Glu-Pro-,
-Ser-Arg-Leu-Glu-Glu-Glu-Leu-Arg-cyclo(Cys-Arg-Leu-hCys)-Glu-Pro-,
-Ser-Arg-Leu-Glu-Glu-Glu-Leu-Arg-cyclo(hCys-Arg-Leu-hCys)-Glu-Pro-,
-Ser-Arg-Leu-Glu-Glu-Glu-Leu-Arg-cyclo(hCys-Arg-Leu-DhCys)-Glu-Pro-,
-Ser-Arg-Leu-Glu-Glu-Glu-Leu-Arg-cyclo(DhCys-Arg-Leu-hCys)-Glu-Pro-,
-Pro-Ser-Arg-Leu-Glu-Glu-Glu-Leu-Arg-Arg-cyclo(Cys-Leu-Thr-DCys)-Pro-,
-Pro-Ser-Arg-Leu-Glu-Glu-Glu-Leu-Arg-Arg-cyclo(DCys-Leu-Thr-Cys)-Pro-,
-Pro-Ser-Arg-Leu-Glu-Glu-Glu-Leu-Arg-Arg-cyclo(DCys-Leu-Thr-DCys)-Pro-,
-Pro-Ser-Arg-Leu-Glu-Glu-Glu-Leu-Arg-Arg-cyclo(hCys-Leu-Thr-Cys)-Pro-,
-Pro-Ser-Arg-Leu-Glu-Glu-Glu-Leu-Arg-Arg-cyclo(hCys-Leu-Thr-DCys)-Pro-,
-Pro-Ser-Arg-Leu-Glu-Glu-Glu-Leu-Arg-Arg-cyclo(hCys-Leu-Thr-hCys)-Pro-,
-Pro-Ser-Arg-Leu-Glu-Glu-Glu-Leu-Arg-Arg-cyclo(Cys-Leu-Thr-hCys)-Pro-,
-Pro-Ser-Arg-Leu-Glu-Glu-Glu-Leu-Arg-Arg-cyclo(DCys-Leu-Thr-hCys)-Pro-,
-Pro-Ser-Arg-Leu-Glu-Glu-Glu-Leu-Arg-Arg-cyclo(Cys-Leu-Thr-hCys)-Pro-,
-Pro-Ser-Arg-Leu-Glu-Glu-Glu-Leu-Arg-Arg-cyclo(DhCys-Leu-Thr-Cys)-Pro-,
-Pro-Ser-Arg-Leu-Glu-Glu-Glu-Leu-Arg-Arg-cyclo(DhCys-Leu-Thr-DCys)-Pro-,
-Pro-Ser-Arg-Leu-Glu-Glu-Glu-Leu-Arg-Arg-cyclo(DhCys-Leu-Thr-hCys)-Pro-,
-Pro-Ser-Arg-Leu-Glu-Glu-Glu-Leu-Arg-Arg-cyclo(Cys-Leu-Thr-DhCys)-Pro-,
-Pro-Ser-Arg-Leu-Glu-Glu-Glu-Leu-Arg-Arg-cyclo(DCys-Leu-Thr-DhCys)-Pro-,
-Pro-Ser-Arg-Leu-Glu-Glu-Glu-Leu-Arg-Arg-cyclo(Cys-Leu-Thr-DhCys)-Pro-,
-Pro-Ser-Arg-Leu-Glu-Glu-Glu-Leu-Arg-Arg-cyclo(Pen-Leu-Thr-Pen)-Pro-,
-Pro-Ser-Arg-Leu-Glu-Glu-Glu-Leu-Arg-Arg-cyclo(Pen-Leu-Thr-DPen)-Pro-,
-Pro-Ser-Arg-Leu-Glu-Glu-Glu-Leu-Arg-Arg-cyclo(DPen-Leu-Thr-Pen)-Pro-,
-Pro-Ser-Arg-Leu-Glu-Glu-Glu-Leu-Arg-Arg-cyclo(DPen-Leu-Thr-DPen)-Pro-,
-Pro-Ser-Arg-Leu-Glu-Glu-Glu-Leu-Arg-cyclo(DCys-Arg-Leu-Cys)-Glu-Pro-,
-Pro-Ser-Arg-Leu-Glu-Glu-Glu-Leu-Arg-cyclo(DCys-Arg-Leu-DCys)-Glu-Pro-,
-Pro-Ser-Arg-Leu-Glu-Glu-Glu-Leu-Arg-cyclo(Cys-Arg-Leu-DCys)-Glu-Pro-,
-Pro-Ser-Arg-Leu-Glu-Glu-Glu-Leu-Arg-cyclo(hCys-Arg-Leu-Cys)-Glu-Pro-,
-Pro-Ser-Arg-Leu-Glu-Glu-Glu-Leu-Arg-cyclo(hCys-Arg-Leu-DCys)-Glu-Pro-,
-Pro-Ser-Arg-Leu-Glu-Glu-Glu-Leu-Arg-cyclo(Cys-Arg-Leu-hCys)-Glu-Pro-,
-Pro-Ser-Arg-Leu-Glu-Glu-Glu-Leu-Arg-cyclo(hCys-Arg-Leu-hCys)-Glu-Pro-,
-Pro-Ser-Arg-Leu-Glu-Glu-Glu-Leu-Arg-cyclo(hCys-Arg-Leu-DhCys)-Glu-Pro-,
-Pro-Ser-Arg-Leu-Glu-Glu-Glu-Leu-Arg-cyclo(DhCys-Arg-Leu-hCys)-Glu-Pro-,
-Ser-Arg-Leu-Glu-Glu-Glu-Leu-Arg-cyclo(Lys-Arg-Leu-Asp)-Glu-Pro-,
-Ser-Arg-Leu-Glu-Glu-Glu-Leu-Arg-cyclo(Asp-Arg-Leu-Lys)-Glu-Pro-,
-Ser-Arg-Leu-Glu-Glu-Glu-Leu-Arg-cyclo(Lys-Arg-Leu-Glu)-Glu-Pro-,
-Ser-Arg-Leu-Glu-Glu-Glu-Leu-Arg-cyclo(Glu-Arg-Leu-Lys)-Glu-Pro-,
-Pro-Ser-Arg-Leu-Glu-Glu-Glu-Leu-Arg-cyclo(Lys-Arg-Leu-Asp)-Glu-Pro-,
-Pro-Ser-Arg-Leu-Glu-Glu-Glu-Leu-Arg-cyclo(Asp-Arg-Leu-Lys)-Glu-Pro-,
-Pro-Ser-Arg-Leu-Glu-Glu-Glu-Leu-Arg-cyclo(Lys-Arg-Leu-Glu)-Glu-Pro-,
-Pro-Ser-Arg-Leu-Glu-Glu-Glu-Leu-Arg-cyclo(Glu-Arg-Leu-Lys)-Glu-Pro-,
-Pro-Ser-Arg-Leu-Glu-Glu-Glu-Leu-Arg-cyclo(Cys-Arg-Leu-Thr-Cys)-Pro-,
-Pro-Ser-Arg-Leu-Glu-cyclo(Cys-Glu-Leu-Arg-Cys)-Arg-Leu-Thr-Glu-Pro-,
-Pro-Ser-Arg-Leu-Glu-Glu-Glu-Leu-Arg-Arg-cyclo(Cys-Leu-Thr-Cys)-Pro-,
-Pro-Ser-Arg-Leu-Glu-Glu-cyclo(Cys-Leu-Arg-Arg-Cys)-Leu-Thr-Glu-Pro-,
-Ser-Arg-Leu-Glu-cyclo(Lys-Glu-Leu-Arg-Glu)-Arg-Leu-Thr-Glu-,
and
-Ser-Arg-Leu-cyclo(Glu-Glu-Glu-Leu-Lys)-Arg-Arg-Leu-Thr-Glu-.

8. The compound of claim 1 wherein the cyclized amino acid sequence is selected from the group consisting of
-Ser-Arg-Leu-Glu-cyclo(Glu-Glu-Leu-Arg-Lys)-Arg-Leu-Thr-Glu-,
-Ser-Arg-Leu-Glu-cyclo(Asp-Glu-Leu-Arg-Lys)-Arg-Leu-Thr-Glu-,
-Ser-Arg-Leu-Glu-cyclo(Glu-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Glu)-,
-Ser-Arg-Leu-Glu-cyclo(Cys-Glu-Leu-Arg-Cys)-Arg-Leu-Thr-Glu-,
-Ser-Arg-Leu-Glu-cyclo(Asp-Glu-Leu-Lys)-Arg-Arg-Leu-Thr-Glu-,
-Ser-Arg-Leu-Glu-Glu-Glu-Leu-Arg-cyclo(Lys-Arg-Leu-Thr-Asp)-,
-Ser-Arg-Leu-Glu-cyclo(Glu-Glu-Leu-Arg-DLys)-Arg-Leu-Thr-Glu-
-Pro-Ser-Arg-Leu-Glu-cyclo(Glu-Glu-Leu-Arg-Lys)-Arg-Leu-Thr-Glu-,
-Pro-Ser-Arg-Leu-Glu-cyclo(DGlu-Glu-Leu-Arg-Lys)-Arg-Leu-Thr-Glu-
-Pro-Ser-Arg-Leu-Glu-cyclo(Glu-Glu-Leu-Arg-DLys)-Arg-Leu-Thr-Glu-
-Pro-Ser-Arg-Leu-Glu-cyclo(Lys-Glu-Leu-Arg-Glu)-Arg-Leu-Thr-Glu-
-Pro-Ser-Arg-Leu-cyclo(Glu-Glu-Glu-Leu-Lys)-Arg-Arg-Leu-Thr-Glu-,
-Pro-Ser-Arg-Leu-Glu-cyclo(Cys-Glu-Leu-DCys)-Arg-Arg-Leu-Thr-Glu-,
-Pro-Ser-Arg-Leu-Glu-Glu-Glu-Leu-Arg-cyclo(Cys-Arg-Leu-Thr-Cys)-,
-Pro-Ser-Arg-Leu-Glu-cyclo(Cys-Glu-Leu-Arg-Cys)-Arg-Leu-Thr-Glu-,
-Pro-Ser-Arg-Leu-Glu-Glu-Glu-Leu-Arg-Arg-cyclo(Cys-Leu-Thr-Cys)-,
-Pro-Ser-Arg-Leu-Glu-Glu-cyclo(Cys-Leu-Arg-Arg-Cys)-Leu-Thr-Glu-,
-Ser-Arg-Leu-Glu-Glu-Glu-Leu-Arg-cyclo(Cys-Arg-Leu-Thr-Cys)-,
-Ser-Arg-Leu-Glu-cyclo(Cys-Glu-Leu-Arg-Cys)-Arg-Leu-Thr-Glu-,
-Ser-Arg-Leu-Glu-cyclo(Cys-Glu-Leu-Cys)-Arg-Arg-Leu-Thr-Glu-,
-Ser-Arg-Leu-Glu-Glu-Glu-Leu-Arg-Arg-cyclo(Cys-Leu-Thr-Cys)-,
-Ser-Arg-Leu-Glu-Glu-cyclo(Cys-Leu-Arg-Arg-Cys)-Leu-Thr-Glu-,
-Ser-Arg-Leu-Glu-cyclo(Lys-Glu-Leu-Arg-Glu)-Arg-Leu-Thr-Glu-,
and
-Ser-Arg-Leu-cyclo(Glu-Glu-Glu-Leu-Lys)-Arg-Arg-Leu-Thr-Glu-.

9. The compound of claim 1 wherein the cyclized amino acid sequence is
(i)
-Ser-Arg-Leu-Glu-cyclo(Glu-Glu-Leu-Arg-Lys)-Arg-Leu-Thr-Glu--;
(ii)
-Ser-Arg-Leu-Glu-cyclo(Asp-Glu-Leu-Arg-Lys)-Arg-Leu-Thr-Glu-;
(iii)
-Ser-Arg-Leu-Glu-cyclo(Glu-Glu-Leu-Lys)-Arg-Arg-Leu-Thr-Glu-;
or
(iv)
-Ser-Arg-Leu-Glu-Glu-Glu-Leu-Arg-cyclo(Lys-Arg-Leu-Thr-Glu)-.

10-12. (canceled)

13. The compound of claim 1 wherein the cyclized amino acid sequence is selected from the group consisting of

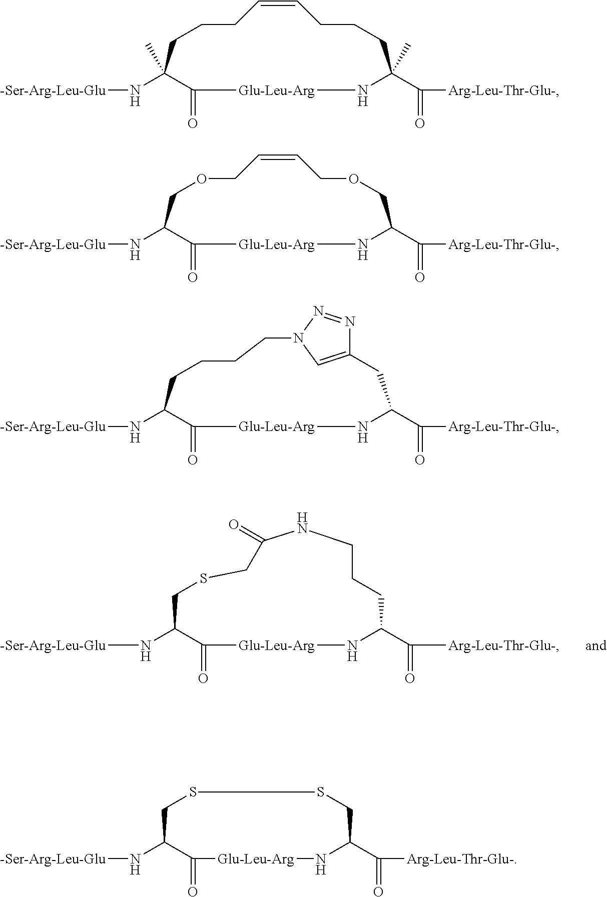

14. The compound of claim 1 wherein the peptide consists of the cyclized amino acid sequence.

15. The compound of claim 1 which is a fusion protein.

16. The compound of claim 15 wherein the fusion protein
(i) comprises the peptide and a further peptide or polypeptide;
(ii) does not comprise non-peptidic components; or
(iii) comprises the peptide and a further peptide or polypeptide, and does not comprise non-peptidic components.

17. (canceled)

18. The compound of claim 1 which comprises one or more non-peptidic components.

19. A complex comprising the compound of claim 1 and a compound comprising a moiety binding to the peptide comprising a cyclized amino acid sequence.

20. The complex of claim 19 wherein the moiety binding to the peptide comprising a cyclized amino acid sequence comprises an antibody or antibody fragment.

21. A method of forming a complex comprising contacting the compound of claim 1 and a compound comprising a moiety binding to the peptide comprising a cyclized amino acid sequence.

22. The method of claim 21 wherein the moiety binding to the peptide comprising a cyclized amino acid sequence comprises an antibody or antibody fragment.

23. A complex comprising the compound of claim 9 and a compound comprising a moiety binding to the peptide comprising a cyclized amino acid sequence.

24. A complex comprising the compound of claim 13 and a compound comprising a moiety binding to the peptide comprising a cyclized amino acid sequence.

25. A method of forming a complex comprising contacting the compound of claim 9 and a compound comprising a moiety binding to the peptide comprising a cyclized amino acid sequence.

26. A method of forming a complex comprising contacting the compound of claim 13 and a compound comprising a moiety binding to the peptide comprising a cyclized amino acid sequence.


SEQUENCE LISTING (366 sequences)

SEQ ID NO: 1 - protein, 13 aa, synthetic construct
  SITE 5..9: note = glutamic acid lysine amide
SRLEEELRKRLTE

SEQ ID NO: 2 - protein, 13 aa, synthetic construct
  SITE 5..9: note = aspartatic acid lysine amide
SRLEDELRKRLTE

SEQ ID NO: 3 - protein, 13 aa, synthetic construct
  SITE 5..8: note = glutamic acid lysine amide
SRLEEELKRRLTE

SEQ ID NO: 4 - protein, 13 aa, synthetic construct
  SITE 9..13: note = lysine glutamic acid amide
SRLEEELRKRLTE

SEQ ID NO: 5 - protein, 13 aa, synthetic construct
  SITE 5..9: note = cysteine cysteine disulfide
SRLECELRCRLTE

SEQ ID NO: 6 - protein, 13 aa, synthetic construct
  SITE 5..8: note = aspartic acid lysine amide
SRLEDELKRRLTE

SEQ ID NO: 7 - protein, 13 aa, synthetic construct
  SITE 9..13: note = lysine aspartic acid amide
SRLEEELRKRLTD

SEQ ID NO: 8 - protein, 13 aa, synthetic construct
  SITE 5..9: note = glutamic acid D-lysine amide
  SITE 9: note = D-lysine
SRLEEELRKRLTE

SEQ ID NO: 9 - protein, 14 aa, synthetic construct
  SITE 6..10: note = glutamic acid lysine amide
PSRLEEELRKRLTE

SEQ ID NO: 10 - protein, 14 aa, synthetic construct
  SITE 6..10: note = D-glutamic acid lysine amide
  SITE 6: note = D-glutamic acid
PSRLEEELRKRLTE

SEQ ID NO: 11 - protein, 14 aa, synthetic construct
  SITE 6..10: note = glutamic acid D-lysine amide
  SITE 10: note = D-lysine
PSRLEEELRKRLTE

SEQ ID NO: 12 - protein, 14 aa, synthetic construct
  SITE 6..10: note = lysine glutamic acid amide
PSRLEKELRERLTE

SEQ ID NO: 13 - protein, 14 aa, synthetic construct
  SITE 5..9: note = glutamic acid lysine amide
PSRLEEELKRRLTE

SEQ ID NO: 14 - protein, 14 aa, synthetic construct
  SITE 6..9: note = cysteine D-cysteine disulfide
  SITE 9: note = D-cysteine
PSRLECELCRRLTE

SEQ ID NO: 15 - protein, 14 aa, synthetic construct
  SITE 10..14: note = cysteine cysteine disulfide
PSRLEEELRCRLTC

SEQ ID NO: 16 - protein, 14 aa, synthetic construct
  SITE 6..10: note = cysteine cysteine disulfide
PSRLECELRCRLTE

SEQ ID NO: 17 - protein, 14 aa, synthetic construct
  SITE 11..14: note = cysteine cysteine disulfide
PSRLEEELRRCLTC

SEQ ID NO: 18 - protein, 14 aa, synthetic construct
  SITE 7..11: note = cysteine cysteine disulfide
PSRLEECLRRCLTE

SEQ ID NO: 19 - protein, 13 aa, synthetic construct
  SITE 9..13: note = cysteine cysteine disulfide
SRLEEELRCRLTC

SEQ ID NO: 20 - protein, 13 aa, synthetic construct
  SITE 5..9: note = cysteine cysteine disulfide
SRLECELRCRLTE

SEQ ID NO: 21 - protein, 13 aa, synthetic construct
  SITE 5..8: note = cysteine cysteine disulfide
SRLECELCRRLTE

SEQ ID NO: 22 - protein, 13 aa, synthetic construct
  SITE 10..13: note = cysteine cysteine disulfide
SRLEEELRRCLTC

SEQ ID NO: 23 - protein, 13 aa, synthetic construct
  SITE 6..10: note = cysteine cysteine disulfide
SRLEECLRRCLTE

SEQ ID NO: 24 - protein, 13 aa, synthetic construct
  SITE 5..9: note = D-glutamic acid D-lysine amide
  SITE 5: note = D-glutamic acid
  SITE 9: note = D-lysine
SRLEEELRKRLTE

SEQ ID NO: 25 - protein, 13 aa, synthetic construct
  SITE 5..9: note = D-lysine glutamic acid amide
  SITE 5: note = D-lysine
SRLEKELRERLTE

SEQ ID NO: 26 - protein, 13 aa, synthetic construct
  SITE 5..9: note = lysine D-glutamic acid amide
  SITE 9: note = D-glutamic acid
SRLEKELRERLTE

SEQ ID NO: 27 - protein, 13 aa, synthetic construct
  SITE 5..9: note = D-lysine D-glutamic acid amide
  SITE 5: note = D-lysine
  SITE 9: note = D-glutamic acid
SRLEKELRERLTE

SEQ ID NO: 28 - protein, 14 aa, synthetic construct
  SITE 6..10: note = D-glutamic acid D-lysine amide
  SITE 6: note = D-glutamic acid
  SITE 10: note = D-lysine
PSRLEEELRKRLTE

SEQ ID NO: 29 - protein, 14 aa, synthetic construct
  SITE 6..10: note = D-lysine glutamic acid amide
  SITE 6: note = D-lysine
PSRLEKELRERLTE

SEQ ID NO: 30 - protein, 14 aa, synthetic construct
  SITE 6..10: note = lysine D-glutamic amide
  SITE 10: note = D-glutamic acid
PSRLEKELRERLTE

SEQ ID NO: 31 - protein, 14 aa, synthetic construct
  SITE 6..10: note = D-lysine D-glutamic acid amide
  SITE 6: note = D-lysine
  SITE 10: note = D-glutamic acid
PSRLEKELRERLTE

SEQ ID NO: 32 - protein, 13 aa, synthetic construct
  SITE 5..9: note = D-lysine aspartic acid amide
  SITE 5: note = D-lysine
SRLEKELRDRLTE

SEQ ID NO: 33 - protein, 13 aa, synthetic construct
  SITE 5..9: note = D-aspartic acid D-lysine amide
  SITE 5: note = D-aspartic acid
  SITE 9: note = D-lysine
SRLEDELRKRLTE

SEQ ID NO: 34 - protein, 13 aa, synthetic construct
  SITE 5..9: note = D-aspartic acid lysine amide
  SITE 5: note = D-aspartic acid
SRLEDELRKRLTE

SEQ ID NO: 35 - protein, 13 aa, synthetic construct
  SITE 5..9: note = aspartic acid D-lysine amide
  SITE 9: note = D-lysine
SRLEDELRKRLTE

SEQ ID NO: 36 - protein, 13 aa, synthetic construct
  SITE 5..9: note = lysine D-aspartic acid amide
  SITE 9: note = D-aspartic acid
SRLEKELRDRLTE

SEQ ID NO: 37 - protein, 13 aa, synthetic construct
  SITE 5..9: note = D-lysine D-aspartic acid amide
  SITE 5: note = D-lysine
  SITE 9: note = D-aspartic acid
SRLEKELRDRLTE

SEQ ID NO: 38 - protein, 14 aa, synthetic construct
  SITE 6..10: note = D-lysine aspartic acid amide
  SITE 6: note = D-lysine
PSRLEKELRDRLTE

SEQ ID NO: 39 - protein, 14 aa, synthetic construct
  SITE 6..10: note = D-aspartic acid D-lysine amide
  SITE 6: note = D-aspartic acid
  SITE 10: note = D-lysine
PSRLEDELRKRLTE

SEQ ID NO: 40 - protein, 14 aa, synthetic construct
  SITE 6..10: note = D-aspartic acid lysine amide
  SITE 6: note = D-aspartic acid
PSRLEDELRKRLTE

SEQ ID NO: 41 - protein, 14 aa, synthetic construct
  SITE 6..10: note = aspartic acid D-lysine amide
  SITE 10: note = D-lysine
PSRLEDELRKRLTE

SEQ ID NO: 42 - protein, 14 aa, synthetic construct
  SITE 6..10: note = lysine D-aspartic acid amide
  SITE 10: note = D-aspartic acid
PSRLEKELRDRLTE

SEQ ID NO: 43 - protein, 14 aa, synthetic construct
  SITE 6..10: note = D-lysine D-aspartic acid amide
  SITE 6: note = D-lysine
  SITE 10: note = D-spartic acid
PSRLEKELRDRLTE

SEQ ID NO: 44 - protein, 13 aa, synthetic construct
  SITE 5..8: note = D-glutamic acid lysine amide
  SITE 5: note = D-glutamic acid
SRLEEELKRRLTE

SEQ ID NO: 45 - protein, 13 aa, synthetic construct
  SITE 5..8: note = glutamic acid D-lysine amide
  SITE 8: note = D-lysine
SRLEEELKRRLTE

SEQ ID NO: 46 - protein, 13 aa, synthetic construct
  SITE 5..8: note = D-glutamic acid D-lysine amide
  SITE 5: note = D-glutamic acid
  SITE 8: note = D-lysine
SRLEEELKRRLTE

SEQ ID NO: 47 - protein, 13 aa, synthetic construct
  SITE 5..8: note = lysine glutamic acid amide
SRLEKELERRLTE

SEQ ID NO: 48 - protein, 13 aa, synthetic construct
  SITE 5..8: note = lysine D-glutamic acid amide
  SITE 8: note = D-glutamic acid
SRLEKELERRLTE

SEQ ID NO: 49 - protein, 13 aa, synthetic construct
  SITE 5..8: note = D-lysine D-glutamic acid amide
  SITE 5: note = D-lysine
  SITE 8: note = D-glutamic acid
SRLEKELERRLTE

SEQ ID NO: 50 - protein, 13 aa, synthetic construct
  SITE 5..8: note = aspartic acid lysine amide
SRLEDELKRRLTE

SEQ ID NO: 51 - protein, 13 aa, synthetic construct
  SITE 5..8: note = aspartic acid D-lysine amide
  SITE 8: note = D-lysine
SRLEDELKRRLTE

SEQ ID NO: 52 - protein, 13 aa, synthetic construct
  SITE 5..8: note = lysine aspartic acid amide
SRLEKELDRRLTE

SEQ ID NO: 53 - protein, 13 aa, synthetic construct
  SITE 5..8: note = lysine D-aspartic acid amide
  SITE 8: note = D-aspartic acid
SRLEKELDRRLTE

SEQ ID NO: 54 - protein, 13 aa, synthetic construct
  SITE 5..8: note = D-aspartic acid lysine amide
  SITE 5: note = D-aspartic acid
SRLEDELKRRLTE

SEQ ID NO: 55 - protein, 13 aa, synthetic construct
  SITE 5..8: note = D-aspartic acid D-lysine amide
  SITE 5: note = D-aspartic acid
  SITE 8: note = D-lysine
SRLEDELKRRLTE

SEQ ID NO: 56 - protein, 13 aa, synthetic construct
  SITE 5..8: note = D-lysine D-aspartic acid amide
  SITE 5: note = D-lysine
  SITE 8: note = D-aspartic acid
SRLEKELDRRLTE

SEQ ID NO: 57 - protein, 13 aa, synthetic construct
  SITE 5..8: note = D-lysine aspartic acid amide
  SITE 5: note = D-lysine
SRLEKELDRRLTE

SEQ ID NO: 58 - protein, 14 aa, synthetic construct
  SITE 6..9: note = glutamic acid D-lysine amide
  SITE 9: note = D-lysine
PSRLEEELKRRLTE

SEQ ID NO: 59 - protein, 14 aa, synthetic construct
  SITE 6..9: note = D-glutamic acid D-lysine amide
  SITE 6: note = D-glutamic acid
  SITE 9: note = D-lysine
PSRLEEELKRRLTE

SEQ ID NO: 60 - protein, 14 aa, synthetic construct
  SITE 6..9: note = lysine glutamic acid amide
PSRLEKELERRLTE

SEQ ID NO: 61 - protein, 14 aa, synthetic construct
  SITE 6..9: note = lysine D-glutamic acid amide
  SITE 9: note = D-glutamic acid
PSRLEKELERRLTE

SEQ ID NO: 62 - protein, 14 aa, synthetic construct
  SITE 6..9: note = D-lysine D-glutamic acid amide
  SITE 6: note = D-lysine
  SITE 9: note = D-glutamic acid
PSRLEKELERRLTE

SEQ ID NO: 63 - protein, 14 aa, synthetic construct
  SITE 6..9: note = aspartic acid lysine amide
PSRLEDELKRRLTE

SEQ ID NO: 64 - protein, 14 aa, synthetic construct
  SITE 6..9: note = aspartic acid D-lysine amide
  SITE 9: note = D-lysine
PSRLEDELKRRLTE

SEQ ID NO: 65 - protein, 14 aa, synthetic construct
  SITE 6..9: note = lysine aspartic acid amide
PSRLEKELDRRLTE

SEQ ID NO: 66 - protein, 14 aa, synthetic construct
  SITE 6..9: note = lysine D-aspartic acid amide
  SITE 9: note = D-aspartic acid
PSRLEKELDRRLTE

SEQ ID NO: 67 - protein, 14 aa, synthetic construct
  SITE 6..9: note = D-aspartic acid lysine amide
  SITE 6: note = D-aspartic acid
PSRLEDELKRRLTE

SEQ ID NO: 68 - protein, 14 aa, synthetic construct
  SITE 6..9: note = D-aspartic acid D-lysine amide
  SITE 6: note = D-aspartic acid
  SITE 9: note = D-lysine
PSRLEDELKRRLTE

SEQ ID NO: 69 - protein, 14 aa, synthetic construct
  SITE 6..9: note = D-lysine D-aspartic acid amide
  SITE 6: note = D-lysine
  SITE 9: note = D-aspartic acid
PSRLEKELDRRLTE

SEQ ID NO: 70 - protein, 14 aa, synthetic construct
  SITE 6..9: note = D-lysine aspartic acid amide
  SITE 6: note = D-lysine
PSRLEKELDRRLTE

SEQ ID NO: 71 - protein, 13 aa, synthetic construct
  SITE 6..9: note = aspartic acid lysine amide
SRLEEDLRKRLTE

SEQ ID NO: 72 - protein, 13 aa, synthetic construct
  SITE 6..9: note = lysine aspartic acid amide
SRLEEKLRDRLTE

SEQ ID NO: 73 - protein, 13 aa, synthetic construct
  SITE 6..9: note = D-aspartic acid lysine amide
  SITE 6: note = D-aspartic acid
SRLEEDLRKRLTE

SEQ ID NO: 74 - protein, 13 aa, synthetic construct
  SITE 6..9: note = lysine D-aspartic acid amide
  SITE 9: note = D-aspartic acid
SRLEEKLRDRLTE

SEQ ID NO: 75 - protein, 13 aa, synthetic construct
  SITE 6..9: note = aspartic acid D-lysine amide
  SITE 9: note = D-lysine
SRLEEDLRKRLTE

SEQ ID NO: 76 - protein, 13 aa, synthetic construct
  SITE 6..9: note = D-lysine aspartic acid amide
  SITE 6: note = D-lysine
SRLEEKLRDRLTE

SEQ ID NO: 77 - protein, 13 aa, synthetic construct
  SITE 6..9: note = D-lysine D-aspartic acid amide
  SITE 6: note = D-lysine
  SITE 9: note = D-aspartic acid
SRLEEKLRDRLTE

SEQ ID NO: 78 - protein, 13 aa, synthetic construct
  SITE 6..9: note = D-aspartic acid D-lysine amide
  SITE 6: note = D-aspartic acid
  SITE 9: note = D-lysine
SRLEEDLRKRLTE

SEQ ID NO: 79 - protein, 14 aa, synthetic construct
  SITE 7..10: note = aspartic acid lysine amide
PSRLEEDLRKRLTE

SEQ ID NO: 80 - protein, 14 aa, synthetic construct
  SITE 7..10: note = lysine aspartic acid amide
PSRLEEKLRDRLTE

SEQ ID NO: 81 - protein, 14 aa, synthetic construct
  SITE 7..10: note = D-aspartic acid lysine amide
  SITE 7: note = D-aspartic acid
PSRLEEDLRKRLTE

SEQ ID NO: 82 - protein, 14 aa, synthetic construct
  SITE 7..10: note = lysine D-aspartic acid amide
  SITE 10: note = D-aspartic acid
PSRLEEKLRDRLTE

SEQ ID NO: 83 - protein, 14 aa, synthetic construct
  SITE 7..10: note = aspartic acid D-lysine amide
  SITE 10: note = D-lysine
PSRLEEDLRKRLTE

SEQ ID NO: 84 - protein, 14 aa, synthetic construct
  SITE 7..10: note = D-lysine aspartic acid amide
  SITE 7: note = D-lysine
PSRLEEKLRDRLTE

SEQ ID NO: 85 - protein, 14 aa, synthetic construct
  SITE 7..10: note = D-lysine D-aspartic acid amide
  SITE 7: note = D-lysine
  SITE 10: note = D-aspartic acid
PSRLEEKLRDRLTE

SEQ ID NO: 86 - protein, 14 aa, synthetic construct
  SITE 7..10: note = D-aspartic acid D-lysine amide
  SITE 7: note = D-aspartic acid
  SITE 10: note = D-lysine
PSRLEEDLRKRLTE

SEQ ID NO: 87 - protein, 13 aa, synthetic construct
  SITE 5..8: note = cysteine cysteine disulfide
SRLECELCRRLTE

SEQ ID NO: 88 - protein, 13 aa, synthetic construct
  SITE 10..13: note = cysteine cysteine disulfide
SRLEEELRRCLTC

SEQ ID NO: 89 - protein, 13 aa, synthetic construct
  SITE 9..12: note = cysteine cysteine disulfide
SRLEEELRCRLCE

SEQ ID NO: 90 - protein, 13 aa, synthetic construct
  SITE 6..9: note = cysteine cysteine disulfide
SRLEECLRCRLTE

SEQ ID NO: 91 - protein, 14 aa, synthetic construct
  SITE 6..9: note = cysteine cysteine disulfide
PSRLECELCRRLTE

SEQ ID NO: 92 - protein, 14 aa, synthetic construct
  SITE 11..14: note = cysteine cysteine disulfide
PSRLEEELRRCLTC

SEQ ID NO: 93 - protein, 14 aa, synthetic construct
  SITE 10..13: note = cysteine cysteine disulfide
PSRLEEELRCRLCE

SEQ ID NO: 94 - protein, 14 aa, synthetic construct
  SITE 7..10: note = cysteine cysteine disulfide
PSRLEECLRCRLTE

SEQ ID NO: 95 - protein, 13 aa, synthetic construct
  SITE 5..8: note = D-cysteine D-cysteine disulfide
  SITE 5: note = D-cysteine
  SITE 8: note = D-cysteine
SRLECELCRRLTE

SEQ ID NO: 96 - protein, 13 aa, synthetic construct
  SITE 5..8: note = D-cysteine cysteine disulfide
  SITE 5: note = D-cysteine
SRLECELCRRLTE

SEQ ID NO: 97 - protein, 13 aa, synthetic construct
  SITE 5..8: note = homocysteine cysteine disulfide
  SITE 5: note = homocysteine
SRLEXELCRRLTE

SEQ ID NO: 98 - protein, 13 aa, synthetic construct
  SITE 5..8: note = homocysteine homocysteine disulfide
  SITE 5: note = homocysteine
  SITE 8: note = homocysteine
SRLEXELXRRLTE

SEQ ID NO: 99 - protein, 13 aa, synthetic construct
  SITE 5..8: note = cysteine homocysteine disulfide
  SITE 8: note = homocysteine
SRLECELXRRLTE

SEQ ID NO: 100 - protein, 13 aa, synthetic construct
  SITE 5..8: note = homocysteine D-cysteine disulfide
  SITE 5: note = homocysteine
  SITE 8: note = D-cysteine
SRLEXELCRRLTE

SEQ ID NO: 101 - protein, 13 aa, synthetic construct
  SITE 5..8: note = D-cysteine homocysteine disulfide
  SITE 5: note = D-cysteine
  SITE 8: note = homocysteine
SRLECELXRRLTE

SEQ ID NO: 102 - protein, 13 aa, synthetic construct
  SITE 5..8: note = homocysteine D-homocysteine disulfide
  SITE 5: note = homocysteine
  SITE 8: note = D-homocysteine
SRLEXELXRRLTE

SEQ ID NO: 103 - protein, 13 aa, synthetic construct
  SITE 5..8: note = D-homocysteine D-homocysteine disulfide
  SITE 5: note = D-homocysteine
  SITE 8: note = D-homocysteine
SRLEXELXRRLTE

SEQ ID NO: 104 - protein, 13 aa, synthetic construct
  SITE 5..8: note = cysteine D-homocysteine disulfide
  SITE 8: note = D-homocysteine
SRLECELXRRLTE

SEQ ID NO: 105 - protein, 13 aa, synthetic construct
  SITE 5..8: note = D-cysteine D-homocysteine disulfide
  SITE 5: note = D-cysteine
  SITE 8: note = D-homocysteine
SRLECELXRRLTE

SEQ ID NO: 106 - protein, 13 aa, synthetic construct
  SITE 5..8: note = D-homocysteine cysteine disulfide
  SITE 5: note = D-homocysteine
SRLEXELCRRLTE

SEQ ID NO: 107 - protein, 13 aa, synthetic construct
  SITE 5..8: note = D-homocysteine D-cysteine disulfide
  SITE 5: note = D-homocysteine
  SITE 8: note = D-cysteine
SRLEXELCRRLTE

SEQ ID NO: 108 - protein, 14 aa, synthetic construct
  SITE 6..9: note = D-cysteine D-cysteine disulfide
  SITE 6: note = D-cysteine
  SITE 9: note = D-cysteine
PSRLECELCRRLTE

SEQ ID NO: 109 - protein, 14 aa, synthetic construct
  SITE 6..9: note = D-cysteine cysteine disulfide
  SITE 6: note = D-cysteine
PSRLECELCRRLTE

SEQ ID NO: 110 - protein, 14 aa, synthetic construct
  SITE 6..9: note = homocysteine cysteine disulfide
  SITE 6: note = homocysteine
PSRLEXELCRRLTE

SEQ ID NO: 111 - protein, 14 aa, synthetic construct
  SITE 6..9: note = homocysteine homocysteine disulfide
  SITE 6: note = homocysteine
  SITE 9: note = homocysteine
PSRLEXELXRRLTE

SEQ ID NO: 112 - protein, 14 aa, synthetic construct
  SITE 6..9: note = cysteine homocysteine disulfide
  SITE 9: note = homocysteine
PSRLECELXRRLTE

SEQ ID NO: 113 - protein, 14 aa, synthetic construct
  SITE 6..9: note = homocysteine D-cysteine disulfide
  SITE 6: note = homocysteine
  SITE 9: note = D-cysteine
PSRLEXELCRRLTE

SEQ ID NO: 114 - protein, 14 aa, synthetic construct
  SITE 6..9: note = D-cysteine homocysteine disulfide
  SITE 6: note = D-cysteine
  SITE 9: note = homocysteine
PSRLECELXRRLTE

SEQ ID NO: 115 - protein, 14 aa, synthetic construct
  SITE 6..9: note = homocysteine D-homocysteine disulfide
  SITE 6: note = homocysteine
  SITE 9: note = D-homocysteine
PSRLEXELXRRLTE

SEQ ID NO: 116 - protein, 14 aa, synthetic construct
  SITE 6..9: note = D-homocysteine D-homocysteine disulfide
  SITE 6: note = D-homocysteine
  SITE 9: note = D-homocysteine
PSRLEXELXRRLTE

SEQ ID NO: 117 - protein, 14 aa, synthetic construct
  SITE 6..9: note = cysteine D-homocysteine disulfide
  SITE 9: note = D-homocysteine
PSRLECELXRRLTE

SEQ ID NO: 118 - protein, 14 aa, synthetic construct
  SITE 6..9: note = D-cysteine D-homocysteine disulfide
  SITE 6: note = D-cysteine
  SITE 9: note = D-homocysteine
PSRLECELXRRLTE

SEQ ID NO: 119 - protein, 14 aa, synthetic construct
  SITE 6..9: note = D-homocysteine cysteine disulfide
  SITE 6: note = D-homocysteine
PSRLEXELCRRLTE

SEQ ID NO: 120 - protein, 14 aa, synthetic construct
  SITE 6..9: note = D-homocysteine D-cysteine disulfide
  SITE 6: note = D-homocysteine
  SITE 9: note = D-cysteine
PSRLEXELCRRLTE

SEQ ID NO: 121 - protein, 13 aa, synthetic construct
  SITE 10..13: note = cysteine D-cysteine disulfide
  SITE 13: note = D-cysteine
SRLEEELRRCLTC

SEQ ID NO: 122 - protein, 13 aa, synthetic construct
  SITE 10..13: note = D-cysteine cysteine disulfide
  SITE 10: note = D-cysteine
SRLEEELRRCLTC

SEQ ID NO: 123 - protein, 13 aa, synthetic construct
  SITE 10..13: note = D-cysteine D-cysteine disulfide
  SITE 10: note = D-cysteine
  SITE 13: note = D-cysteine
SRLEEELRRCLTC

SEQ ID NO: 124 - protein, 13 aa, synthetic construct
  SITE 10..13: note = homocysteine cysteine disulfide
  SITE 10: note = homocysteine
SRLEEELRRXLTC

SEQ ID NO: 125 - protein, 13 aa, synthetic construct
  SITE 10..13: note = homocysteine D-cysteine disulfide
  SITE 10: note = homocysteine
  SITE 13: note = D-cysteine
SRLEEELRRXLTC

SEQ ID NO: 126 - protein, 13 aa, synthetic construct
  SITE 10..13: note = homocysteine homocysteine disulfide
  SITE 10: note = homocysteine
  SITE 13: note = homocysteine
SRLEEELRRXLTX

SEQ ID NO: 127 - protein, 13 aa, synthetic construct
  SITE 10..13: note = cysteine homocysteine disulfide
  SITE 13: note = homocysteine
SRLEEELRRCLTX

SEQ ID NO: 128 - protein, 13 aa, synthetic construct
  SITE 10..13: note = D-cysteine homocysteine disulfide
  SITE 10: note = D-cysteine
  SITE 13: note = homocysteine
SRLEEELRRCLTX

SEQ ID NO: 129 - protein, 13 aa, synthetic construct
  SITE 10..13: note = cysteine homocysteine disulfide
  SITE 13: note = homocysteine
SRLEEELRRCLTX

SEQ ID NO: 130 - protein, 13 aa, synthetic construct
  SITE 10..13: note = D-homocysteine cysteine disulfide
  SITE 10: note = D-homocysteine
SRLEEELRRXLTC

SEQ ID NO: 131 - protein, 13 aa, synthetic construct
  SITE 10..13: note = D-homocysteine D-cysteine disulfide
  SITE 10: note = D-homocysteine
  SITE 13: note = D-cysteine
SRLEEELRRXLTC

SEQ ID NO: 132 - protein, 13 aa, synthetic construct
  SITE 10..13: note = D-homocysteine homocysteine disulfide
  SITE 10: note = D-homocysteine
  SITE 13: note = homocysteine
SRLEEELRRXLTX

SEQ ID NO: 133 - protein, 13 aa, synthetic construct
  SITE 10..13: note = cysteine D-homocysteine disulfide
  SITE 13: note = D-homocysteine
SRLEEELRRCLTX

SEQ ID NO: 134 - protein, 13 aa, synthetic construct
  SITE 10..13: note = D-cysteine D-homocysteine disulfide
  SITE 10: note = D-cysteine
  SITE 13: note = D-homocysteine
SRLEEELRRCLTX

SEQ ID NO: 135 - protein, 13 aa, synthetic construct
  SITE 10..13: note = cysteine D-homocysteine disulfide
  SITE 13: note = D-homocysteine
SRLEEELRRCLTX

SEQ ID NO: 136 - protein, 13 aa, synthetic construct
  SITE 10..13: note = penicillamine penicillamine disulfide
  SITE 10: note = penicillamine
  SITE 13: note = penicillamine
SRLEEELRRXLTX

SEQ ID NO: 137 - protein, 13 aa, synthetic construct
  SITE 10..13: note = penicillamine D-penicillamine disulfide
  SITE 10: note = penicillamine
  SITE 13: note = D-penicillamine
SRLEEELRRXLTX

SEQ ID NO: 138 - protein, 13 aa, synthetic construct
  SITE 10..13: note = D-penicillamine penicillamine disulfide
  SITE 10: note = D-penicillamine
  SITE 13: note = penicillamine
SRLEEELRRXLTX

SEQ ID NO: 139 - protein, 13 aa, synthetic construct
  SITE 10..13: note = D-penicillamine D-penicillamine disulfide
  SITE 10: note = D-penicillamine
  SITE 13: note = D-penicillamine
SRLEEELRRXLTX

SEQ ID NO: 140 - protein, 13 aa, synthetic construct
  SITE 9..12: note = D-cysteine cysteine disulfide
  SITE 9: note = D-cysteine
SRLEEELRCRLCE

SEQ ID NO: 141 - protein, 13 aa, synthetic construct
  SITE 9..12: note = D-cysteine D-cysteine disulfide
  SITE 9: note = D-cysteine
  SITE 12: note = D-cysteine
SRLEEELRCRLCE

SEQ ID NO: 142 - protein, 13 aa, synthetic construct
  SITE 9..12: note = cysteine D-cysteine disulfide
  SITE 12: note = D-cysteine
SRLEEELRCRLCE

SEQ ID NO: 143 - protein, 13 aa, synthetic construct
  SITE 9..12: note = homocysteine cysteine disulfide
  SITE 9: note = homocysteine
SRLEEELRXRLCE

SEQ ID NO: 144 - protein, 13 aa, synthetic construct
  SITE 9..12: note = homocysteine D-cysteine disulfide
  SITE 9: note = homocysteine
  SITE 12: note = D-cysteine
SRLEEELRXRLCE

SEQ ID NO: 145 - protein, 13 aa, synthetic construct
  SITE 9..12: note = cysteine homocysteine disulfide
  SITE 12: note = homocysteine
SRLEEELRCRLXE

SEQ ID NO: 146 - protein, 13 aa, synthetic construct
  SITE 9..12: note = homocysteine homocysteine disulfide
  SITE 9: note = homocysteine
  SITE 12: note = homocysteine
SRLEEELRXRLXE

SEQ ID NO: 147 - protein, 13 aa, synthetic construct
  SITE 9..12: note = homocysteine D-homocysteine disulfide
  SITE 9: note = homocysteine
  SITE 12: note = D-homocysteine
SRLEEELRXRLXE

SEQ ID NO: 148 - protein, 13 aa, synthetic construct
  SITE 9..12: note = D-homocysteine homocysteine disulfide
  SITE 9: note = D-homocysteine
  SITE 12: note = homocysteine
SRLEEELRXRLXE

SEQ ID NO: 149 - protein, 14 aa, synthetic construct
  SITE 11..14: note = cysteine D-cysteine disulfide
  SITE 14: note = D-cysteine
PSRLEEELRRCLTC

SEQ ID NO: 150 - protein, 14 aa, synthetic construct
  SITE 11..14: note = D-cysteine cysteine disulfide
  SITE 11: note = D-cysteine
PSRLEEELRRCLTC

SEQ ID NO: 151 - protein, 14 aa, synthetic construct
  SITE 11..14: note = D-cysteine D-cysteine disulfide
  SITE 11: note = D-cysteine
  SITE 14: note = D-cysteine
PSRLEEELRRCLTC

SEQ ID NO: 152 - protein, 14 aa, synthetic construct
  SITE 11..14: note = homocysteine cysteine disulfide
  SITE 11: note = homocysteine
PSRLEEELRRXLTC

SEQ ID NO: 153 - protein, 14 aa, synthetic construct
  SITE 11..14: note = homocysteine D-cysteine disulfide
  SITE 11: note = homocysteine
  SITE 14: note = D-cysteine
PSRLEEELRRXLTC

SEQ ID NO: 154 - protein, 14 aa, synthetic construct
  SITE 11..14: note = homocysteine homocysteine disulfide
  SITE 11: note = homocysteine
  SITE 14: note = homocysteine
PSRLEEELRRXLTX

SEQ ID NO: 155 - protein, 14 aa, synthetic construct
  SITE 11..14: note = cysteine homocysteine disulfide
  SITE 14: note = homocysteine
PSRLEEELRRCLTX

SEQ ID NO: 156 - protein, 14 aa, synthetic construct
  SITE 11..14: note = D-cysteine homocysteine disulfide
  SITE 11: note = D-cysteine
  SITE 14: note = homocysteine
PSRLEEELRRCLTX

SEQ ID NO: 157 - protein, 14 aa, synthetic construct
  SITE 11..14: note = cysteine homocysteine disulfide
  SITE 14: note = homocysteine
PSRLEEELRRCLTX

SEQ ID NO: 158 - protein, 14 aa, synthetic construct
  SITE 11..14: note = D-homocysteine cysteine disulfide
  SITE 11: note = D-homocysteine
PSRLEEELRRXLTC

SEQ ID NO: 159 - protein, 14 aa, synthetic construct
  SITE 11..14: note = D-homocysteine D-cysteine disulfide
  SITE 11: note = D-homocysteine
  SITE 14: note = D-cysteine
PSRLEEELRRXLTC

SEQ ID NO: 160 - protein, 14 aa, synthetic construct
  SITE 11..14: note = D-homocysteine homocysteine disulfide
  SITE 11: note = D-homocysteine
  SITE 14: note = homocysteine
PSRLEEELRRXLTX

SEQ ID NO: 161 - protein, 14 aa, synthetic construct
  SITE 11..14: note = cysteine D-homocysteine disulfide
  SITE 14: note = D-homocysteine
PSRLEEELRRCLTX

SEQ ID NO: 162 - protein, 14 aa, synthetic construct
  SITE 11..14: note = D-cysteine D-homocysteine disulfide
  SITE 11: note = D-cysteine
  SITE 14: note = D-homocysteine
PSRLEEELRRCLTX

SEQ ID NO: 163 - protein, 14 aa, synthetic construct
  SITE 11..14: note = cysteine D-homocysteine disulfide
  SITE 14: note = D-homocysteine
PSRLEEELRRCLTX

SEQ ID NO: 164 - protein, 14 aa, synthetic construct
  SITE 11..14: note = penicillamine penicillamine disulfide
  SITE 11: note = penicillamine
  SITE 14: note = penicillamine
PSRLEEELRRXLTX

SEQ ID NO: 165 - protein, 14 aa, synthetic construct
  SITE 11..14: note = penicillamine D-penicillamine disulfide
  SITE 11: note = penicillamine
  SITE 14: note = D-penicillamine
PSRLEEELRRXLTX

SEQ ID NO: 166 - protein, 14 aa, synthetic construct
  SITE 11..14: note = D-penicillamine penicillamine diulfide
  SITE 11: note = D-penicillamine
  SITE 14: note = penicillamine
PSRLEEELRRXLTX

SEQ ID NO: 167 - protein, 14 aa, synthetic construct
  SITE 11..14: note = D-penicillamine D-penicillamine disulfide
  SITE 11: note = D-penicillamine
  SITE 14: note = D-penicillamine
PSRLEEELRRXLTX

SEQ ID NO: 168 - protein, 14 aa, synthetic construct
  SITE 10..13: note = D-cysteine cysteine disulfide
  SITE 10: note = D-cysteine
PSRLEEELRCRLCE

SEQ ID NO: 169 - protein, 14 aa, synthetic construct
  SITE 10..13: note = D-cysteine D-cysteine disulfide
  SITE 10: note = D-cysteine
  SITE 13: note = D-cysteine
PSRLEEELRCRLCE

SEQ ID NO: 170 - protein, 14 aa, synthetic construct
  SITE 10..13: note = cysteine D-cysteine disulfide
  SITE 13: note = D-cysteine
PSRLEEELRCRLCE

SEQ ID NO: 171 - protein, 14 aa, synthetic construct
  SITE 10..13: note = homocysteine cysteine disulfide
  SITE 10: note = homocysteine
PSRLEEELRXRLCE

SEQ ID NO: 172 - protein, 14 aa, synthetic construct
  SITE 10..13: note = homocysteine D-cysteine disulfide
  SITE 10: note = homocysteine
  SITE 13: note = D-cysteine
PSRLEEELRXRLCE

SEQ ID NO: 173 - protein, 14 aa, synthetic construct
  SITE 10..13: note = cysteine homocysteine disulfide
  SITE 13: note = homocysteine
PSRLEEELRCRLXE

SEQ ID NO: 174 - protein, 14 aa, synthetic construct
  SITE 10..13: note = homocysteine homocysteine disulfide
  SITE 10: note = homocysteine
  SITE 13: note = homocysteine
PSRLEEELRXRLXE

SEQ ID NO: 175 - protein, 14 aa, synthetic construct
  SITE 10..13: note = homocysteine D-homocysteine disulfide
  SITE 10: note = homocysteine
  SITE 13: note = D-homocysteine
PSRLEEELRXRLXE

SEQ ID NO: 176 - protein, 14 aa, synthetic construct
  SITE 10..13: note = D-homocysteine homocysteine disulfide
  SITE 10: note = D-homocysteine
  SITE 13: note = homocysteine
PSRLEEELRXRLXE

SEQ ID NO: 177 - protein, 13 aa, synthetic construct
  SITE 9..12: note = lysine aspartic acid amide
SRLEEELRKRLDE

SEQ ID NO: 178 - protein, 13 aa, synthetic construct
  SITE 9..12: note = aspartic acid lysine amide
SRLEEELRDRLKE

SEQ ID NO: 179 - protein, 13 aa, synthetic construct
  SITE 9..12: note = lysine glutamic acid amide
SRLEEELRKRLEE

SEQ ID NO: 180 - protein, 13 aa, synthetic construct
  SITE 9..12: note = glutamic acid lysine amide
SRLEEELRERLKE

SEQ ID NO: 181 - protein, 14 aa, synthetic construct
  SITE 10..13: note = lysine aspartic acid amide
PSRLEEELRKRLDE

SEQ ID NO: 182 - protein, 14 aa, synthetic construct
  SITE 10..13: note = aspartic acid lysine amide
PSRLEEELRDRLKE

SEQ ID NO: 183 - protein, 14 aa, synthetic construct
  SITE 10..13: note = lysine glutamic acid amide
PSRLEEELRKRLEE

SEQ ID NO: 184 - protein, 14 aa, synthetic construct
  SITE 10..13: note = glutamic acid lysine amide
PSRLEEELRERLKE

SEQ ID NO: 185 - protein, 14 aa, synthetic construct
  SITE 10..14: note = cysteine cysteine disulfide
PSRLEEELRCRLTC

SEQ ID NO: 186 - protein, 14 aa, synthetic construct
  SITE 6..10: note = cysteine cysteine disulfide
PSRLECELRCRLTE

SEQ ID NO: 187 - protein, 14 aa, synthetic construct
  SITE 11..14: note = cysteine cysteine disulfide
PSRLEEELRRCLTC

SEQ ID NO: 188 - protein, 14 aa, synthetic construct
  SITE 7..11: note = cysteine cysteine disulfide
PSRLEECLRRCLTE

SEQ ID NO: 189 - protein, 14 aa, synthetic construct
  SITE 5..9: note = D-glutamic acid D-lysine amide
  SITE 5: note = D-glutamic acid
  SITE 9: note = D-lysine
SRLEEELRKRLTEP

SEQ ID NO: 190 - protein, 14 aa, synthetic construct
  SITE 5..9: note = D-lysine glutamic acid amide
  SITE 5: note = D-lysine
SRLEKELRERLTEP

SEQ ID NO: 191 - protein, 14 aa, synthetic construct
  SITE 5..9: note = lysine D-glutamic acid amide
  SITE 9: note = D-glutamic acid
SRLEKELRERLTEP

SEQ ID NO: 192 - protein, 14 aa, synthetic construct
  SITE 5..9: note = D-lysine D-glutamic acid amide
  SITE 5: note = D-lysine
  SITE 9: note = D-glutamic acid
SRLEKELRERLTEP

SEQ ID NO: 193 - protein, 15 aa, synthetic construct
  SITE 6..10: note = D-glutamic acid D-lysine amide
  SITE 6: note = D-glutamic acid
  SITE 10: note = D-lysine
PSRLEEELRKRLTEP

SEQ ID NO: 194 - protein, 15 aa, synthetic construct
  SITE 6..10: note = D-lysine glutamic acid amide
  SITE 6: note = D-lysine
PSRLEKELRERLTEP

SEQ ID NO: 195 - protein, 15 aa, synthetic construct
  SITE 6..10: note = lysine D-glutamic acid amide
  SITE 10: note = D-glutamic acid
PSRLEKELRERLTEP

SEQ ID NO: 196 - protein, 15 aa, synthetic construct
  SITE 6..10: note = D-lysine D-glutamic acid amide
  SITE 6: note = D-lysine
  SITE 10: note = D-glutamic acid
PSRLEKELRERLTEP

SEQ ID NO: 197 - protein, 14 aa, synthetic construct
  SITE 5..9: note = D-lysine aspartic acid amide
  SITE 5: note = D-lysine
SRLEKELRDRLTEP

SEQ ID NO: 198 - protein, 14 aa, synthetic construct
  SITE 5..9: note = D-aspartic acid D-lysine amide
  SITE 5: note = D-aspartic acid
  SITE 9: note = D-lysine
SRLEDELRKRLTEP

SEQ ID NO: 199 - protein, 14 aa, synthetic construct
  SITE 5..9: note = D-aspartic acid lysine amide
  SITE 5: note = D-aspartic acid
SRLEDELRKRLTEP

SEQ ID NO: 200 - protein, 14 aa, synthetic construct
  SITE 5..9: note = aspartic acid D-lysine amide
  SITE 9: note = D-lysine
SRLEDELRKRLTEP

SEQ ID NO: 201 - protein, 14 aa, synthetic construct
  SITE 5..9: note = lysine D-aspartic acid amide
  SITE 9: note = D-aspartic acid
SRLEKELRDRLTEP

SEQ ID NO: 202 - protein, 14 aa, synthetic construct
  SITE 5..9: note = D-lysine D-aspartic acid amide
  SITE 5: note = D-lysine
  SITE 9: note = D-aspartic acid
SRLEKELRDRLTEP

SEQ ID NO: 203 - protein, 15 aa, synthetic construct
  SITE 6..10: note = D-lysine aspartic acid amide
  SITE 6: note = D-lysine
PSRLEKELRDRLTEP

SEQ ID NO: 204 - protein, 15 aa, synthetic construct
  SITE 6..10: note = D-aspartic acid D-lysine amide
  SITE 6: note = D-aspartic acid
  SITE 10: note = D-lysine
PSRLEDELRKRLTEP

SEQ ID NO: 205 - protein, 15 aa, synthetic construct
  SITE 6..10: note = D-aspartic acid lysine amide
  SITE 6: note = D-aspartic acid
PSRLEDELRKRLTEP

SEQ ID NO: 206 - protein, 15 aa, synthetic construct
  SITE 6..10: note = aspartic acid D-lysine amide
  SITE 10: note = D-lysine
PSRLEDELRKRLTEP

SEQ ID NO: 207 - protein, 15 aa, synthetic construct
  SITE 6..10: note = lysine D-aspartic acid amide
  SITE 10: note = D-aspartic acid
PSRLEKELRDRLTEP

SEQ ID NO: 208 - protein, 15 aa, synthetic construct
  SITE 6..10: note = D-lysine D-aspartic acid amide
  SITE 6: note = D-lysine
  SITE 10: note = D-aspartic acid
PSRLEKELRDRLTEP

SEQ ID NO: 209 - protein, 14 aa, synthetic construct
  SITE 5..8: note = D-glutamic acid lysine amide
  SITE 5: note = D-glutamic acid
SRLEEELKRRLTEP

SEQ ID NO: 210 - protein, 14 aa, synthetic construct
  SITE 5..8: note = glutamic acid D-lysine amide
  SITE 8: note = D-lysine
SRLEEELKRRLTEP

SEQ ID NO: 211 - protein, 14 aa, synthetic construct
  SITE 5..8: note = D-glutamic acid D-lysine amide
  SITE 5: note = D-glutamic acid
  SITE 8: note = D-lysine
SRLEEELKRRLTEP

SEQ ID NO: 212 - protein, 14 aa, synthetic construct
  SITE 5..8: note = lysine glutamic acid amide
SRLEKELERRLTEP

SEQ ID NO: 213 - protein, 14 aa, synthetic construct
  SITE 5..8: note = lysine D-glutamic acid amide
  SITE 8: note = D-glutamic acid
SRLEKELERRLTEP

SEQ ID NO: 214 - protein, 14 aa, synthetic construct
  SITE 5..8: note = D-lysine D-glutamic acid amide
  SITE 5: note = D-lysine
  SITE 8: note = D-glutamic acid
SRLEKELERRLTEP

SEQ ID NO: 215 - protein, 14 aa, synthetic construct
  SITE 5..8: note = aspartic acid lysine amide
SRLEDELKRRLTEP

SEQ ID NO: 216 - protein, 14 aa, synthetic construct
  SITE 5..8: note = aspartic acid D-lysine amide
  SITE 8: note = D-lysine
SRLEDELKRRLTEP

SEQ ID NO: 217 - protein, 14 aa, synthetic construct
  SITE 5..8: note = lysine aspartic acid amide
SRLEKELDRRLTEP

SEQ ID NO: 218 - protein, 14 aa, synthetic construct
  SITE 5..8: note = lysine D-aspartic acid amide
  SITE 8: note = D-aspartic acid
SRLEKELDRRLTEP

SEQ ID NO: 219 - protein, 14 aa, synthetic construct
  SITE 5..8: note = D-aspartic acid lysine amide
  SITE 5: note = D-aspartic acid
SRLEDELKRRLTEP

SEQ ID NO: 220 - protein, 14 aa, synthetic construct
  SITE 5..8: note = D-aspartic acid D-lysine amide
  SITE 5: note = D-aspartic acid
  SITE 8: note = D-lysine
SRLEDELKRRLTEP

SEQ ID NO: 221 - protein, 14 aa, synthetic construct
  SITE 5..8: note = D-lysine D-aspartic acid amide
  SITE 5: note = D-lysine
  SITE 8: note = D-aspartic acid
SRLEKELDRRLTEP

SEQ ID NO: 222 - protein, 14 aa, synthetic construct
  SITE 5..8: note = D-lysine aspartic acid amide
  SITE 5: note = D-lysine
SRLEKELDRRLTEP

SEQ ID NO: 223 - protein, 15 aa, synthetic construct
  SITE 6..9: note = glutamic acid D-lysine amide
  SITE 9: note = D-lysine
PSRLEEELKRRLTEP

SEQ ID NO: 224 - protein, 15 aa, synthetic construct
  SITE 6..9: note = D-glutamic acid D-lysine amide
  SITE 6: note = D-glutamic acid
  SITE 9: note = D-lysine
PSRLEEELKRRLTEP

SEQ ID NO: 225 - protein, 15 aa, synthetic construct
  SITE 6..9: note = lysine glutamic acid amide
PSRLEKELERRLTEP

SEQ ID NO: 226 - protein, 15 aa, synthetic construct
  SITE 6..9: note = lysine D-glutamic acid amide
  SITE 9: note = D-glutamic acid
PSRLEKELERRLTEP

SEQ ID NO: 227 - protein, 15 aa, synthetic construct
  SITE 6..9: note = D-lysine D-glutamic acid amide
  SITE 6: note = D-lysine
  SITE 9: note = D-glutamic acid
PSRLEKELERRLTEP

SEQ ID NO: 228 - protein, 15 aa, synthetic construct
  SITE 6..9: note = aspartic acid lysine amide
PSRLEDELKRRLTEP

SEQ ID NO: 229 - protein, 15 aa, synthetic construct
  SITE 6..9: note = aspartic acid D-lysine amide
  SITE 9: note = D-lysine
PSRLEDELKRRLTEP

SEQ ID NO: 230 - protein, 15 aa, synthetic construct
  SITE 6..9: note = lysine aspartic acid amide
PSRLEKELDRRLTEP

SEQ ID NO: 231 - protein, 15 aa, synthetic construct
  SITE 6..9: note = lysine D-aspartic acid amide
  SITE 9: note = D-aspartic acid
PSRLEKELDRRLTEP

SEQ ID NO: 232 - protein, 15 aa, synthetic construct
  SITE 6..9: note = D-aspartic acid lysine amide
  SITE 6: note = D-aspartic acid
PSRLEDELKRRLTEP

SEQ ID NO: 233 - protein, 15 aa, synthetic construct
  SITE 6..9: note = D-aspartic acid D-lysine amide
  SITE 6: note = D-aspartic acid
  SITE 9: note = D-lysine
PSRLEDELKRRLTEP

SEQ ID NO: 234 - protein, 15 aa, synthetic construct
  SITE 6..9: note = D-lysine D-aspartic acid amide
  SITE 6: note = D-lysine
  SITE 9: note = D-aspartic acid
PSRLEKELDRRLTEP

SEQ ID NO: 235 - protein, 15 aa, synthetic construct
  SITE 6..9: note = D-lysine aspartic acid amide
  SITE 6: note = D-lysine
PSRLEKELDRRLTEP

SEQ ID NO: 236 - protein, 14 aa, synthetic construct
  SITE 6..9: note = aspartic acid lysine amide
SRLEEDLRKRLTEP

SEQ ID NO: 237 - protein, 14 aa, synthetic construct
  SITE 6..9: note = lysine aspartic acid amide
SRLEEKLRDRLTEP

SEQ ID NO: 238 - protein, 14 aa, synthetic construct
  SITE 6..9: note = D-aspartic acid lysine amide
  SITE 6: note = D-aspartic acid
SRLEEDLRKRLTEP

SEQ ID NO: 239 - protein, 14 aa, synthetic construct
  SITE 6..9: note = lysine D-aspartic acid amide
  SITE 9: note = D-aspartic acid
SRLEEKLRDRLTEP

SEQ ID NO: 240 - protein, 14 aa, synthetic construct
  SITE 6..9: note = aspartic acid D-lysine amide
  SITE 9: note = D-lysine
SRLEEDLRKRLTEP

SEQ ID NO: 241 - protein, 14 aa, synthetic construct
  SITE 6..9: note = D-lysine aspartic acid amide
  SITE 6: note = D-lysine
SRLEEKLRDRLTEP

SEQ ID NO: 242 - protein, 14 aa, synthetic construct
  SITE 6..9: note = D-lysine D-aspartic acid amide
  SITE 6: note = D-lysine
  SITE 9: note = D-aspartic acid
SRLEEKLRDRLTEP

SEQ ID NO: 243 - protein, 14 aa, synthetic construct
  SITE 6..9: note = D-aspartic acid D-lysine amide
  SITE 6: note = D-aspartic acid
  SITE 9: note = D-lysine
SRLEEDLRKRLTEP

SEQ ID NO: 244 - protein, 15 aa, synthetic construct
  SITE 7..10: note = aspartic acid lysine amide
PSRLEEDLRKRLTEP

SEQ ID NO: 245 - protein, 15 aa, synthetic construct
  SITE 7..10: note = lysine aspartic acid amide
PSRLEEKLRDRLTEP

SEQ ID NO: 246 - protein, 15 aa, synthetic construct
  SITE 7..10: note = D-aspartic acid lysine amide
  SITE 7: note = D-aspartic acid
PSRLEEDLRKRLTEP

SEQ ID NO: 247 - protein, 15 aa, synthetic construct
  SITE 7..10: note = lysine D-aspartic acid amide
  SITE 10: note = D-aspartic acid
PSRLEEKLRDRLTEP

SEQ ID NO: 248 - protein, 15 aa, synthetic construct
  SITE 7..10: note = aspartic acid D-lysine amide
  SITE 10: note = D-lysine
PSRLEEDLRKRLTEP

SEQ ID NO: 249 - protein, 15 aa, synthetic construct
  SITE 7..10: note = D-lysine aspartic acid amide
  SITE 7: note = D-lysine
PSRLEEKLRDRLTEP

SEQ ID NO: 250 - protein, 15 aa, synthetic construct
  SITE 7..10: note = D-lysine D-aspartic acid amide
  SITE 7: note = D-lysine
  SITE 10: note = D-aspartic acid
PSRLEEKLRDRLTEP

SEQ ID NO: 251 - protein, 15 aa, synthetic construct
  SITE 7..10: note = D-aspartic acid D-lysine amide
  SITE 7: note = D-aspartic acid
  SITE 10: note = D-lysine
PSRLEEDLRKRLTEP

SEQ ID NO: 252 - protein, 14 aa, synthetic construct
  SITE 5..8: note = cysteine cysteine disulfide
SRLECELCRRLTEP

SEQ ID NO: 253 - protein, 14 aa, synthetic construct
  SITE 10..13: note = cysteine cysteine disulfide
SRLEEELRRCLTCP

SEQ ID NO: 254 - protein, 14 aa, synthetic construct
  SITE 9..12: note = cysteine cysteine disulfide
SRLEEELRCRLCEP

SEQ ID NO: 255 - protein, 14 aa, synthetic construct
  SITE 6..9: note = cysteine cysteine disulfide
SRLEECLRCRLTEP

SEQ ID NO: 256 - protein, 15 aa, synthetic construct
  SITE 6..9: note = cysteine cysteine disulfide
PSRLECELCRRLTEP

SEQ ID NO: 257 - protein, 15 aa, synthetic construct
  SITE 11..14: note = cysteine cysteine disulfide
PSRLEEELRRCLTCP

SEQ ID NO: 258 - protein, 15 aa, synthetic construct
  SITE 10..13: note = cysteine cysteine disulfide
PSRLEEELRCRLCEP

SEQ ID NO: 259 - protein, 15 aa, synthetic construct
  SITE 7..10: note = cysteine cysteine disulfide
PSRLEECLRCRLTEP

SEQ ID NO: 260 - protein, 14 aa, synthetic construct
  SITE 5..8: note = D-cysteine D-cysteine disulfide
  SITE 5: note = D-cysteine
  SITE 8: note = D-cysteine
SRLECELCRRLTEP

SEQ ID NO: 261 - protein, 14 aa, synthetic construct
  SITE 5..8: note = D-cysteine cysteine disulfide
  SITE 5: note = D-cysteine
SRLECELCRRLTEP

SEQ ID NO: 262 - protein, 14 aa, synthetic construct
  SITE 5..8: note = homocysteine cysteine disulfide
  SITE 5: note = homocysteine
SRLEXELCRRLTEP

SEQ ID NO: 263 - protein, 14 aa, synthetic construct
  SITE 5..8: note = homocysteine homocysteine disulfide
  SITE 5: note = homocysteine
  SITE 8: note = homocysteine
SRLEXELXRRLTEP

SEQ ID NO: 264 - protein, 14 aa, synthetic construct
  SITE 5..8: note = cysteine homocysteine disulfide
  SITE 8: note = homocysteine
SRLECELXRRLTEP

SEQ ID NO: 265 - protein, 14 aa, synthetic construct
  SITE 5..8: note = homocysteine D-cysteine disulfide
  SITE 5: note = homocysteine
  SITE 8: note = D-cysteine
SRLEXELCRRLTEP

SEQ ID NO: 266 - protein, 14 aa, synthetic construct
  SITE 5..8: note = D-cysteine homocysteine disulfide
  SITE 5: note = D-cysteine
  SITE 8: note = homocysteine
SRLECELXRRLTEP

SEQ ID NO: 267 - protein, 14 aa, synthetic construct
  SITE 5..8: note = homocysteine D-homocysteine disulfide
  SITE 5: note = homocysteine
  SITE 8: note = D-homocysteine
SRLEXELXRRLTEP

SEQ ID NO: 268 - protein, 14 aa, synthetic construct
  SITE 5..8: note = D-homocysteine D-homocysteine disulfide
  SITE 5: note = D-homocysteine
  SITE 8: note = D-homocysteine
SRLEXELXRRLTEP

SEQ ID NO: 269 - protein, 14 aa, synthetic construct
  SITE 5..8: note = cysteine D-homocysteine disulfide
  SITE 8: note = D-homocysteine
SRLECELXRRLTEP

SEQ ID NO: 270 - protein, 14 aa, synthetic construct
  SITE 5..8: note = D-cysteine D-homocysteine disulfide
  SITE 5: note = D-cysteine
  SITE 8: note = D-homocysteine
SRLECELXRRLTEP

SEQ ID NO: 271 - protein, 14 aa, synthetic construct
  SITE 5..8: note = D-homocysteine cysteine disulfide
  SITE 5: note = D-homocysteine
SRLEXELCRRLTEP

SEQ ID NO: 272 - protein, 14 aa, synthetic construct
  SITE 5..8: note = D-homocysteine D-cysteine disulfide
  SITE 5: note = D-homocysteine
  SITE 8: note = D-cysteine
SRLEXELCRRLTEP

SEQ ID NO: 273 - protein, 15 aa, synthetic construct
  SITE 6..9: note = D-cysteine D-cysteine disulfide
  SITE 6: note = D-cysteine
  SITE 9: note = D-cysteine
PSRLECELCRRLTEP

SEQ ID NO: 274 - protein, 15 aa, synthetic construct
  SITE 6..9: note = D-cysteine cysteine disulfide
  SITE 6: note = D-cysteine
PSRLECELCRRLTEP

SEQ ID NO: 275 - protein, 15 aa, synthetic construct
  SITE 6..9: note = homocysteine cysteine disulfide
  SITE 6: note = homocysteine
PSRLEXELCRRLTEP

SEQ ID NO: 276 - protein, 15 aa, synthetic construct
  SITE 6..9: note = homocysteine homocysteine disulfide
  SITE 6: note = homocysteine
  SITE 9: note = homocysteine
PSRLEXELXRRLTEP

SEQ ID NO: 277 - protein, 15 aa, synthetic construct
  SITE 6..9: note = cysteine homocysteine disulfide
  SITE 9: note = homocysteine
PSRLECELXRRLTEP

SEQ ID NO: 278 - protein, 15 aa, synthetic construct
  SITE 6..9: note = homocysteine D-cysteine disulfide
  SITE 6: note = homocysteine
  SITE 9: note = D-cysteine
PSRLEXELCRRLTEP

SEQ ID NO: 279 - protein, 15 aa, synthetic construct
  SITE 6..9: note = D-cysteine homocysteine disulfide
  SITE 6: note = D-cysteine
  SITE 9: note = homocysteine
PSRLECELXRRLTEP

SEQ ID NO: 280 - protein, 15 aa, synthetic construct
  SITE 6..9: note = homocysteine D-homocysteine disulfide
  SITE 6: note = homocysteine
  SITE 9: note = D-homocysteine
PSRLEXELXRRLTEP

SEQ ID NO: 281 - protein, 15 aa, synthetic construct
  SITE 6..9: note = D-homocysteine D-homocysteine disulfide
  SITE 6: note = D-homocysteine
  SITE 9: note = D-homocysteine
PSRLEXELXRRLTEP

SEQ ID NO: 282 - protein, 15 aa, synthetic construct
  SITE 6..9: note = cysteine D-homocysteine disulfide
  SITE 9: note = D-homocysteine
PSRLECELXRRLTEP

SEQ ID NO: 283 - protein, 15 aa, synthetic construct
  SITE 6..9: note = D-cysteine D-homocysteine disulfide
  SITE 6: note = D-cysteine
  SITE 9: note = D-homocysteine
PSRLECELXRRLTEP

SEQ ID NO: 284 - protein, 15 aa, synthetic construct
  SITE 6..9: note = D-homocysteine cysteine disulfide
  SITE 6: note = D-homocysteine
PSRLEXELCRRLTEP

SEQ ID NO: 285 - protein, 15 aa, synthetic construct
  SITE 6..9: note = D-homocysteine D-cysteine disulfide
  SITE 6: note = D-homocysteine
  SITE 9: note = D-cysteine
PSRLEXELCRRLTEP

SEQ ID NO: 286 - protein, 14 aa, synthetic construct
  SITE 10..13: note = cysteine D-cysteine disulfide
  SITE 13: note = D-cysteine
SRLEEELRRCLTCP

SEQ ID NO: 287 - protein, 14 aa, synthetic construct
  SITE 10..13: note = D-cysteine cysteine disulfide
  SITE 10: note = D-cysteine
SRLEEELRRCLTCP

SEQ ID NO: 288 - protein, 14 aa, synthetic construct
  SITE 10..13: note = D-cysteine D-cysteine disulfide
  SITE 10: note = D-cysteine
  SITE 13: note = D-cysteine
SRLEEELRRCLTCP

SEQ ID NO: 289 - protein, 14 aa, synthetic construct
  SITE 10..13: note = homocysteine cysteine disulfide
  SITE 10: note = homocysteine
SRLEEELRRXLTCP

SEQ ID NO: 290 - protein, 14 aa, synthetic construct
  SITE 10..13: note = homocysteine D-cysteine disulfide
  SITE 10: note = homocysteine
  SITE 13: note = D-cysteine
SRLEEELRRXLTCP

SEQ ID NO: 291 - protein, 14 aa, synthetic construct
  SITE 10..13: note = homocysteine homocysteine disulfide
  SITE 10: note = homocysteine
  SITE 13: note = homocysteine
SRLEEELRRXLTXP

SEQ ID NO: 292 - protein, 14 aa, synthetic construct
  SITE 10..13: note = cysteine homocysteine disulfide
  SITE 13: note = homocysteine
SRLEEELRRCLTXP

SEQ ID NO: 293 - protein, 14 aa, synthetic construct
  SITE 10..13: note = D-cysteine homocysteine disulfide
  SITE 10: note = D-cysteine
  SITE 13: note = homocysteine
SRLEEELRRCLTXP

SEQ ID NO: 294 - protein, 14 aa, synthetic construct
  SITE 10..13: note = cysteine homocysteine disulfide
  SITE 13: note = homocysteine
SRLEEELRRCLTXP

SEQ ID NO: 295 - protein, 14 aa, synthetic construct
  SITE 10..13: note = D-homocysteine cysteine disulfide
  SITE 10: note = D-homocysteine
SRLEEELRRXLTCP

SEQ ID NO: 296 - protein, 14 aa, synthetic construct
  SITE 10..13: note = D-homocysteine D-cysteine disulfide
  SITE 13: note = D-cysteine
  SITE 10: note = D-homocysteine
SRLEEELRRXLTCP

SEQ ID NO: 297 - protein, 14 aa, synthetic construct
  SITE 10..13: note = D-homocysteine homocysteine disulfide
  SITE 10: note = D-homocysteine
  SITE 13: note = homocysteine
SRLEEELRRXLTXP

SEQ ID NO: 298 - protein, 14 aa, synthetic construct
  SITE 10..13: note = cysteine D-homocysteine disulfide
  SITE 13: note = D-homocysteine
SRLEEELRRCLTXP

SEQ ID NO: 299 - protein, 14 aa, synthetic construct
  SITE 10..13: note = D-cysteine D-homocysteine disulfide
  SITE 10: note = D-cysteine
  SITE 13: note = D-homocysteine
SRLEEELRRCLTXP

SEQ ID NO: 300 - protein, 14 aa, synthetic construct
  SITE 10..13: note = cysteine D-homocysteine disulfide
  SITE 13: note = D-homocysteine
SRLEEELRRCLTXP

SEQ ID NO: 301 - protein, 14 aa, synthetic construct
  SITE 10..13: note = penicillamine penicillamine disulfide
  SITE 10: note = penicillamine
  SITE 13: note = penicillamine
SRLEEELRRXLTXP

SEQ ID NO: 302 - protein, 14 aa, synthetic construct
  SITE 10..13: note = penicillamine D-penicillamine disulfide
  SITE 10: note = penicillamine
  SITE 13: note = D-penicillamine
SRLEEELRRXLTXP

SEQ ID NO: 303 - protein, 14 aa, synthetic construct
  SITE 10..13: note = D-penicillamine penicillamine disulfide
  SITE 10: note = D-penicillamine
  SITE 13: note = penicillamine
SRLEEELRRXLTXP

SEQ ID NO: 304 - protein, 14 aa, synthetic construct
  SITE 10..13: note = D-penicillamine D-penicillamine disulfide
  SITE 10: note = D-penicillamine
  SITE 13: note = D-penicillamine
SRLEEELRRXLTXP

SEQ ID NO: 305 - protein, 14 aa, synthetic construct
  SITE 9..12: note = D-cysteine cysteine disulfide
  SITE 9: note = D-cysteine
SRLEEELRCRLCEP

SEQ ID NO: 306 - protein, 14 aa, synthetic construct
  SITE 9..12: note = D-cysteine D-cysteine disulfide
  SITE 9: note = D-cysteine
  SITE 12: note = D-cysteine
SRLEEELRCRLCEP

SEQ ID NO: 307 - protein, 14 aa, synthetic construct
  SITE 9..12: note = cysteine D-cysteine disulfide
  SITE 12: note = D-cysteine
SRLEEELRCRLCEP

SEQ ID NO: 308 - protein, 14 aa, synthetic construct
  SITE 9..12: note = homocysteine cysteine disulfide
  SITE 9: note = homocysteine
SRLEEELRXRLCEP

SEQ ID NO: 309 - protein, 14 aa, synthetic construct
  SITE 9..12: note = homocysteine D-cysteine disulfide
  SITE 9: note = homocysteine
  SITE 12: note = D-cysteine
SRLEEELRXRLCEP

SEQ ID NO: 310 - protein, 14 aa, synthetic construct
  SITE 9..12: note = cysteine homocysteine disulfide
  SITE 12: note = homocysteine
SRLEEELRCRLXEP

SEQ ID NO: 311 - protein, 14 aa, synthetic construct
  SITE 9..12: note = homocysteine homocysteine disulfide
  SITE 9: note = homocysteine
  SITE 12: note = homocysteine
SRLEEELRXRLXEP

SEQ ID NO: 312 - protein, 14 aa, synthetic construct
  SITE 9..12: note = homocysteine D-homocysteine disulfide
  SITE 12: note = D-homocysteine
  SITE 9: note = homocysteine
SRLEEELRXRLXEP

SEQ ID NO: 313 - protein, 14 aa, synthetic construct
  SITE 9..12: note = D-homocysteine homocysteine disulfide
  SITE 9: note = D-homocysteine
  SITE 12: note = homocysteine
SRLEEELRXRLXEP

SEQ ID NO: 314 - protein, 15 aa, synthetic construct
  SITE 11..14: note = cysteine D-cysteine disulfide
  SITE 14: note = D-cysteine
PSRLEEELRRCLTCP

SEQ ID NO: 315 - protein, 15 aa, synthetic construct
  SITE 11..14: note = D-cysteine cysteine disulfide
  SITE 11: note = D-cysteine
PSRLEEELRRCLTCP

SEQ ID NO: 316 - protein, 15 aa, synthetic construct
  SITE 11..14: note = D-cysteine D-cysteine disulfide
  SITE 11: note = D-cysteine
  SITE 14: note = D-cysteine
PSRLEEELRRCLTCP

SEQ ID NO: 317 - protein, 15 aa, synthetic construct
  SITE 11..14: note = homocysteine cysteine disulfide
  SITE 11: note = homocysteine
PSRLEEELRRXLTCP

SEQ ID NO: 318 - protein, 15 aa, synthetic construct
  SITE 11..14: note = homocysteine D-cysteine disulfide
  SITE 11: note = homocysteine
  SITE 14: note = D-cysteine
PSRLEEELRRXLTCP

SEQ ID NO: 319 - protein, 15 aa, synthetic construct
  SITE 11..14: note = homocysteine homocysteine disulfide
  SITE 11: note = homocysteine
  SITE 14: note = homocysteine
PSRLEEELRRXLTXP

SEQ ID NO: 320 - protein, 15 aa, synthetic construct
  SITE 11..14: note = cysteine homocysteine disulfide
  SITE 14: note = homocysteine
PSRLEEELRRCLTXP

SEQ ID NO: 321 - protein, 15 aa, synthetic construct
  SITE 11..14: note = D-cysteine homocysteine disulfide
  SITE 11: note = D-cysteine
  SITE 14: note = homocysteine
PSRLEEELRRCLTXP

SEQ ID NO: 322 - protein, 15 aa, synthetic construct
  SITE 11..14: note = cysteine homocysteine disulfide
  SITE 14: note = homocysteine
PSRLEEELRRCLTXP

SEQ ID NO: 323 - protein, 15 aa, synthetic construct
  SITE 11..14: note = D-homocysteine cysteine disulfide
  SITE 11: note = D-homocysteine
PSRLEEELRRXLTCP

SEQ ID NO: 324 - protein, 15 aa, synthetic construct
  SITE 11..14: note = D-homocysteine D-cysteine disulfide
  SITE 11: note = D-homocysteine
  SITE 14: note = D-cysteine
PSRLEEELRRXLTCP

SEQ ID NO: 325 - protein, 15 aa, synthetic construct
  SITE 11..14: note = D-homocysteine homocysteine disulfide
  SITE 11: note = D-homocysteine
  SITE 14: note = homocysteine
PSRLEEELRRXLTXP

SEQ ID NO: 326 - protein, 15 aa, synthetic construct
  SITE 11..14: note = cysteine D-homocysteine disulfide
  SITE 14: note = D-homocysteine
PSRLEEELRRCLTXP

SEQ ID NO: 327 - protein, 15 aa, synthetic construct
  SITE 11..14: note = D-cysteine D-homocysteine disulfide
  SITE 14: note = D-homocysteine
  SITE 11: note = D-cysteine
PSRLEEELRRCLTXP

SEQ ID NO: 328 - protein, 15 aa, synthetic construct
  SITE 11..14: note = cysteine D-homocysteine disulfide
  SITE 14: note = D-homocysteine
PSRLEEELRRCLTXP

SEQ ID NO: 329 - protein, 15 aa, synthetic construct
  SITE 11..14: note = penicillamine penicillamine disulfide
  SITE 11: note = penicillamine
  SITE 14: note = penicillamine
PSRLEEELRRXLTXP

SEQ ID NO: 330 - protein, 15 aa, synthetic construct
  SITE 11..14: note = penicillamine D-penicillamine disulfide
  SITE 11: note = penicillamine
  SITE 14: note = D-penicillamine
PSRLEEELRRXLTXP

SEQ ID NO: 331 - protein, 15 aa, synthetic construct
  SITE 11..14: note = D-penicillamine penicillamine disulfide
  SITE 11: note = D-penicillamine
  SITE 14: note = penicillamine
PSRLEEELRRXLTXP

SEQ ID NO: 332 - protein, 15 aa, synthetic construct
  SITE 11..14: note = D-penicillamine D-penicillamine disulfide
  SITE 11: note = D-penicillamine
  SITE 14: note = D-penicillamine
PSRLEEELRRXLTXP

SEQ ID NO: 333 - protein, 15 aa, synthetic construct
  SITE 10..13: note = D-cysteine cysteine disulfide
  SITE 10: note = D-cysteine
PSRLEEELRCRLCEP

SEQ ID NO: 334 - protein, 15 aa, synthetic construct
  SITE 10..13: note = D-cysteine D-cysteine disulfide
  SITE 10: note = D-cysteine
  SITE 13: note = D-cysteine
PSRLEEELRCRLCEP

SEQ ID NO: 335 - protein, 15 aa, synthetic construct
  SITE 10..13: note = cysteine D-cysteine disulfide
  SITE 13: note = D-cysteine
PSRLEEELRCRLCEP

SEQ ID NO: 336 - protein, 15 aa, synthetic construct
  SITE 10..13: note = homocysteine cysteine disulfide
  SITE 10: note = homocysteine
PSRLEEELRXRLCEP

SEQ ID NO: 337 - protein, 15 aa, synthetic construct
  SITE 10..13: note = homocysteine D-cysteine disulfide
  SITE 10: note = homocysteine
  SITE 13: note = D-cysteine
PSRLEEELRXRLCEP

SEQ ID NO: 338 - protein, 15 aa, synthetic construct
  SITE 10..13: note = cysteine homocysteine disulfide
  SITE 13: note = homocysteine
PSRLEEELRCRLXEP

SEQ ID NO: 339 - protein, 15 aa, synthetic construct
  SITE 10..13: note = homocysteine homocysteine disulfide
  SITE 10: note = homocysteine
  SITE 13: note = homocysteine
PSRLEEELRXRLXEP

SEQ ID NO: 340 - protein, 15 aa, synthetic construct
  SITE 10..13: note = homocysteine D-homocysteine disulfide
  SITE 10: note = homocysteine
  SITE 13: note = D-homocysteine
PSRLEEELRXRLXEP

SEQ ID NO: 341 - protein, 15 aa, synthetic construct
  SITE 10..13: note = D-homocysteine homocysteine disulfide
  SITE 10: note = D-homocysteine
  SITE 13: note = homocysteine
PSRLEEELRXRLXEP

SEQ ID NO: 342 - protein, 14 aa, synthetic construct
  SITE 9..12: note = lysine aspartic acid amide
SRLEEELRKRLDEP

SEQ ID NO: 343 - protein, 14 aa, synthetic construct
  SITE 9..12: note = aspartic acid lysine amide
SRLEEELRDRLKEP

SEQ ID NO: 344 - protein, 14 aa, synthetic construct
  SITE 9..12: note = lysine glutamic acid amide
SRLEEELRKRLEEP

SEQ ID NO: 345 - protein, 14 aa, synthetic construct
  SITE 9..12: note = glutamic acid lysine amide
SRLEEELRERLKEP

SEQ ID NO: 346 - protein, 15 aa, synthetic construct
  SITE 10..13: note = lysine aspartic acid amide
PSRLEEELRKRLDEP

SEQ ID NO: 347 - protein, 15 aa, synthetic construct
  SITE 10..13: note = aspartic acid lysine amide
PSRLEEELRDRLKEP

SEQ ID NO: 348 - protein, 15 aa, synthetic construct
  SITE 10..13: note = lysine glutamic acid amide
PSRLEEELRKRLEEP

SEQ ID NO: 349 - protein, 15 aa, synthetic construct
  SITE 10..13: note = glutamic acid lysine amide
PSRLEEELRERLKEP

SEQ ID NO: 350 - protein, 15 aa, synthetic construct
  SITE 10..14: note = cysteine cysteine disulfide
PSRLEEELRCRLTCP

SEQ ID NO: 351 - protein, 15 aa, synthetic construct
  SITE 6..10: note = cysteine cysteine disulfide
PSRLECELRCRLTEP

SEQ ID NO: 352 - protein, 15 aa, synthetic construct
  SITE 11..14: note = cysteine cysteine disulfide
PSRLEEELRRCLTCP

SEQ ID NO: 353 - protein, 15 aa, synthetic construct
  SITE 7..11: note = cysteine cysteine disulfide
PSRLEECLRRCLTEP

SEQ ID NO: 354 - protein, 13 aa, synthetic construct
  SITE 5: note = residues at positions 5 and 9 form different rings
  SITE 9: note = residues at positions 5 and 9 form different rings
SRLEXELRXRLTE

SEQ ID NO: 355 - protein, 14 aa, synthetic construct
PSRLEEELRRRLTE

SEQ ID NO: 356 - protein, 13 aa, synthetic construct
SRLEEELRRRLTE

SEQ ID NO: 357 - protein, 12 aa, synthetic construct
  VARIANT 3: note = isoleucine or valine
VTXSALNAMAMG

SEQ ID NO: 358 - protein, 9 aa, synthetic construct
  VARIANT 4: note = glutamic acid, histidine, asparagine, aspartic acid or serine
AVSXRGNAM

SEQ ID NO: 359 - protein, 11 aa, synthetic construct
LEDRVDSFHDY

SEQ ID NO: 360 - protein, 13 aa, synthetic construct
  VARIANT 4: note = isoleucine or valine
GVTXSALNAMAMG

SEQ ID NO: 361 - protein, 12 aa, synthetic construct
VTISALNAMAMG

SEQ ID NO: 362 - protein, 9 aa, synthetic construct
AVSERGNAM

SEQ ID NO: 363 - protein, 13 aa, synthetic construct
GVTISALNAMAMG

SEQ ID NO: 364 - protein, 122 aa, synthetic construct
EVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS

SEQ ID NO: 365 - protein, 13 aa, synthetic construct
  SITE 5..9: note = lysine glutamic acid amide
SRLEKELRERLTE

SEQ ID NO: 366 - protein, 13 aa, synthetic construct
  SITE 4..8: note = glutamic acid lysine amide
SRLEEELKRRLTE